(19) United States
(12) Patent Application Publication
(10) Pub. No.: US 2024/0402174 A1
(43) Pub. Date: Dec. 5, 2024

(54) METHODS FOR DETERMINING NOROVIRUS-REACTIVE ANTIBODIES

(71) Applicant: Takeda Vaccines, Inc., Cambridge, MA (US)

(72) Inventors: Ralph BRAUN, Stow, MA (US); Amanda BRINKMAN, Dedham, MA (US); Apurva KULKARNI, Wayland, MA (US)

(21) Appl. No.: 18/698,330

(22) PCT Filed: Oct. 4, 2022
(86) PCT No.: PCT/US2022/077540

§ 371 Date: Apr. 3, 2024

Related U.S. Application Data
(60) Provisional application No. 63/252,053, filed on Oct. 4, 2021.

(51) Int. Cl. G01N 33/569; C12N 7/00; G01N 33/543; G01N 33/58

(52) U.S. Cl. CPC G01N 33/56983; C12N 7/00; G01N 33/54326; G01N 33/582; C12N 2770/16023; G01N 2333/08; G01N 2469/20

(57) ABSTRACT

The present disclosure is directed to methods for determining the presence and/or amount of norovirus-reactive antibodies in a sample from a subject. The subject may be vaccinated with a norovirus vaccine or infected with a norovirus. The present disclosure further relates to in vitro methods for diagnosing a norovirus infection and determining protection against a norovirus infection in a subject for instance after vaccination with a norovirus vaccine. The present disclosure is further directed to kits for determining norovirus-reactive antibodies in a sample. The present disclosure is further directed to microsphere complexes comprising microspheres coupled to norovirus virus like particles.

### CROSS REFERENCE TO RELATED APPLICATIONS

This application claims priority to U.S. Provisional Application No. 63/252,053 filed Oct. 4, 2021, the contents of which are incorporated herein by reference in their entirety.

### FIELD OF THE INVENTION

The present disclosure relates to methods for determining norovirus-reactive antibodies. The present disclosure further relates to in vitro methods for diagnosing a norovirus infection and determining protection against a norovirus infection.

### REFERENCE TO AN ELECTRONIC SEQUENCE LISTING

The contents of the electronic sequence listing submitted electronically herewith (LIGO_031_00WO_SeqList_ST26.xml; Size: 45,085 bytes; and Date of Creation: Oct. 3, 2022) are herein incorporated by reference in their entirety.

### BACKGROUND OF THE INVENTION

Noroviruses are highly prevalent pathogens associated with one fifth of diarrheal disease worldwide, causing more than 200,000 deaths each year. Noroviruses infect people of all age groups, and even though the infection is usually acute and self-limiting, disease can become life threatening in children, the elderly, and immunocompromised people of all age groups. Besides affecting the individual health, the worldwide economic cost caused by norovirus infection and disease is estimated to be around 60 billion dollars per year (Haynes et al., Viruses 2019, 11, 392, doi:10.3390/v11050392).

Noroviruses are single-stranded, positive sense RNA viruses of the family of Caliciviridae that contain a non-segmented genome. The genome of most noroviruses encodes three open reading frames (ORF1, ORF2, and ORF3), except for murine noroviruses which contain a fourth ORF. While ORF1 encodes for nonstructural proteins, the latter two specify the production of the major viral capsid protein VP1 and the minor viral capsid protein VP2. VP1 has shell (S), which surrounds the viral RNA and protruding (P) domains. The P domain, which consists of the highly variable P2 sub-domain is linked to the S domain by a flexible hinge region. The P2 sub-domain harbors major neutralization epitopes and interacts with histo-blood group antigens (HBGAs; Hutson et al., J Virol 2003, 77:405-415).

Noroviruses are genetically and antigenically divergent and are currently classified phylogenetically into 7 different genogroups (GI-GVII) and at least 41 different genotypes. Viruses of GI, GII, and GIV infect humans, and viruses from GI and GII account for nearly all human infections. Since the mid-1990s, viruses from genogroup II genotype 4 (GII.4) have caused the majority of outbreaks, with new variants emerging every 2-3 years. Moreover, GII.4-associated disease has been reported to be more severe than that caused by other genotypes. In 2012, the GII.4/Sydney variant emerged and since then has continued to predominate amongst circulating variants (Vinje, J Clin Microbiol 2015, 53:373-381). The chronological emergence of new GII.4 variants correlates with an increasing number of global epidemics which supports the fact that GII.4 variants may evolve similarly to Influenza A virus, where old variants are periodically replaced.

Although there have been numerous attempts to cultivate human noroviruses in vitro, none of them resulted in the establishment of a robust and reproducible system of viral growth. Therefore, two common vaccine platforms—attenuated or killed viral vaccines—have not been available for developing norovirus vaccines. However, the discovery of norovirus virus like particles (VLPs) has opened new doors for studying norovirus infection, disease and vaccine production. When expressed at high levels in eukaryotic expression systems, the VP1 protein of norovirus self-assembles into VLPs that structurally mimic native norovirus virions. VLPs preserve the authentic confirmation of the viral capsid protein while lacking the infectious genetic material. When viewed by transmission electron microscopy, the VLPs are morphologically indistinguishable from infectious virions isolated from human stool samples. Consequently, VLPs are capable of mimicking the functional interactions of the virus with cellular receptors, thereby eliciting an appropriate host immune response while lacking the ability to reproduce or cause infection. Therefore, most vaccine candidates are based on different variations of VLPs. Due to the high diversity of noroviruses, a major goal in norovirus vaccine development is to prepare formulations that induce cross-reactive antibody responses capable of broad neutralization of multiple genotypes. Cross-challenging studies with chimpanzees demonstrated that animals vaccinated with GII.4 VLP were not protected against infection when challenged with GI.1 norovirus (Bok et al., Proc Natl Acad Sci USA 2011, 108(1):325-30). In view of this, Takeda Vaccines has been developing a bivalent VLP-based vaccine against norovirus that includes VLPs representing each of the major human genogroups. The genogroup I (GI) VLP is based on the Norwalk virus, the first identified norovirus, and is genotype 1 (GI.1). The second VLP is a “consensus” GII.4 VLP made by combining sequences from the Houston (2002), Yerseke (2006) and Den Haag (2006) GII.4 variants (Parra et al., Vaccine 2012, 30, 3580-3586; Treanor et al., J. Infect. Dis. 2014, 210, 1763-1771). GII.4 Consensus VLPs induced high Ab titers against a panel of VLPs of different GII.4 variants that circulated over a period of thirty years (Parra et al., Vaccines 2012, 30(24):3580-3586).

Although promising vaccine formulations have been developed, the measurement of antibody responses upon vaccination or antibody responses after natural infection still remains challenging. Although several assay set-ups such as neutralizing and blockade assays have been developed, these assays are time-consuming, complex, expensive, and still limited to certain types of noroviruses.

In general, neutralization assays measuring antibodies that stop viruses from infecting cells are most commonly used to evaluate the efficacy of vaccines. Organoid-based infection systems enabling cultivation of human norovirus by the application of human intestinal enteroids (HIEs) have only recently become available (Ettayebi et al., Science 2016, 353, 1387-1393; Jones et al., Science 2014, 346, 755-759). Although these systems have been valuable to characterize neutralizing human monoclonal antibodies (mAbs; Alvadro et al., Gastroenterology 2018, 155(6):1898-1907) they are still highly variable and expensive and are not yet suitable to establish neutralization assays with the reproducibility and reliability needed for the routine sample testing. Moreover, these infection systems are not easy to handle and need to be adopted for each norovirus strain that shall be examined. Further, the neutralization assay requires an infrastructure that for most laboratories, let alone hospitals, may not be available, as the test uses live virus and organoids.

Noroviruses have been shown to interact with cell surface carbohydrates, such as HBGAs that have been identified as important attachment factors (Singh et al., J. Virol. 2015, 89, 2024-2040). Blockade assays using norovirus VLPs as substitutes for live viruses have been developed as a surrogate for virus neutralization assays. These assays measure the ability of serum antibodies to block the binding of norovirus VLPs to cell surface carbohydrates. Although the correlates of norovirus immunity in humans are barely understood, blocking antibodies have been found to be a correlate of protection in human infection and challenge models, where vaccinated human subjects were challenged with infectious amounts of viral RNA (Atmar et al., Clinical and Vaccine Immunology 2015, 22(8):923-929; Ramani et al., PLOS Pathogens 2016, 12(4):e1005334). The substrate of choice for VLP binding in blockade assays is pig gastric mucin (PGM) as it contains several human HBGAs. However, blockade assays are expensive and still lack sufficient reproducibility. Moreover, a big limitation is that not all norovirus VLPs are able to bind PGM, which excludes several norovirus strains from diagnosis (Lindesmith et al., J. Virol. 2012, 86, 873-883).

In view of the above, there is an urgent need for reliable, fast, and inexpensive methods for determining norovirus-reactive antibodies which are applicable to any norovirus strain.

Numerous references are cited throughout this specification. Each of the herein cited references is individually incorporated herein by reference in its entirety. In case of conflict, the disclosure of this specification prevails.

### OBJECTS AND SUMMARY

It is an object of the present disclosure to provide a method for determining the presence and/or amount of norovirus-reactive antibodies.

It is a further object of the present disclosure to provide a method for determining the presence and/or amount of norovirus-reactive antibodies, the method providing good reproducibility, high accuracy and high precision.

It is a further object of the present disclosure to provide a method for determining the presence and/or amount of norovirus-reactive antibodies, the method providing good sensitivity.

It is a further object of the present disclosure to provide a method for determining the presence and/or amount of norovirus-reactive antibodies, the method providing the possibility for high-throughput application, due to cost-effectiveness and short turnaround times.

It is a further object of the present disclosure to provide a method for determining the presence and/or amount of norovirus-reactive antibodies, the method providing the possibility to monitor the quality of an immune response after vaccination against one or more noroviruses.

It is a further object of the present disclosure to provide a method for determining the presence and/or amount of norovirus-reactive antibodies, the method not being limited to specific norovirus strains.

It is a further object of the present disclosure to provide a method for determining the presence and/or amount of norovirus-reactive antibodies, the method providing the possibility to distinguish between norovirus-reactive antibodies from different isotypes such as IgA, IgM, and IgG.

It is a further object of the present disclosure to provide a method for determining the presence and/or amount of norovirus-reactive antibodies, the method providing the possibility to determine the presence and/or amount of antibodies reactive against multiple different noroviruses such as noroviruses of genogroup I and genogroup II in one single experiment.

It is a further object of the present disclosure to provide a method for determining the presence and/or amount of norovirus-reactive antibodies, the method providing the possibility to determine norovirus-neutralizing and/or norovirus-blocking antibodies.

It is a further object of the present disclosure to provide an in vitro method for diagnosing a norovirus infection.

It is a further object of the present disclosure to provide an in vitro method for determining protection against a norovirus infection.

It is a further object of the present disclosure to provide a binding partner to which norovirus VLPs can be immobilized to.

It is a further object of the present disclosure to provide a kit for determining norovirus-reactive antibodies.

The present disclosure is therefore directed to a microsphere complex comprising a microsphere coupled to a norovirus VLP.

The present disclosure is therefore further directed to a kit comprising an amount of at least one microsphere complex as described above and optionally an amount of a detection antibody. In addition, the present disclosure is therefore further directed to a kit comprising an amount of at least one microsphere complex as described above and optionally an amount of at least one reporter antibody that binds to the norovirus VLP of the at least one microsphere complex.

The present disclosure is therefore further directed to a method for determining the presence and/or amount of norovirus-reactive antibodies in a sample from a subject, wherein the method comprises the steps of:
Step 1: contacting an amount of a microsphere complex as described above with the sample to allow binding of the norovirus-reactive antibodies in the sample to the norovirus virus like particles (VLPs) coupled to the microspheres in the microsphere complex,
Step 2: contacting an amount of a detection antibody with the norovirus-reactive antibodies bound to the norovirus VLPs in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a detectable label, and
Step 3: detecting a signal from the detection antibody bound to the norovirus-reactive antibodies in step 2,
and wherein the method optionally comprises the further steps of:
Step 4: determining the presence and/or amount of the detection antibody bound to the norovirus-reactive antibodies from the signal of step 3, and
Step 5: determining the presence and/or amount of norovirus-reactive antibodies in the sample from the presence and/or amount of the detection antibody determined in step 4.

The present disclosure is thus further directed to a method for determining the presence and/or amount of norovirus-reactive antibodies in a sample from a subject, wherein the method comprises the steps of:
Step 1: contacting an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label,
with the sample to allow binding of the norovirus-reactive antibodies in the sample to the first and/or the second norovirus VLP,
Step 2: contacting an amount of a detection antibody with the norovirus-reactive antibodies bound to the first and/or the second norovirus VLP in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a third detectable label,
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the detection antibody bound to the norovirus-reactive antibodies bound to the norovirus VLP of the at least one microsphere in step 2 upon irradiation with a second light source,
Step 4: repeating step 3 until at least 30 microspheres coupled to the same norovirus VLP are identified, and
Step 5: summarizing the detected signal from the detection antibody in step 3 for all identified microspheres coupled to the same norovirus VLP, wherein the summarized signal is indicative for the presence and/or amount of norovirus-reactive antibodies in the sample,
and wherein the method optionally comprises the further steps of:
Step 6: determining the presence and/or amount of the detection antibody bound to the norovirus-reactive antibodies from the summarized signal of step 5, and
Step 7: determining the presence and/or amount of norovirus-reactive antibodies in the sample from the presence and/or amount of the detection antibody determined in step 6.

The present disclosure is therefore further directed to a method for determining the presence and/or amount of norovirus-reactive antibodies in a sample from a subject, wherein the method comprises the steps of:
Step 1: providing a kit, including an amount of a microsphere complex as described above and an amount of a reporter antibody as described above, wherein the reporter antibody binds to the norovirus VLP of the microsphere complex,
Step 2: contacting the amount of the microsphere complex and the amount of the reporter antibody with the sample to allow binding of the norovirus-reactive antibodies in the sample to the norovirus VLPs coupled to the microspheres in the microsphere complex while competing with the reporter antibody, and
Step 3: detecting a signal from the reporter antibody bound to the norovirus VLPs in step 2,
and wherein the method optionally comprises the further steps of:
Step 4: determining the presence and/or amount of the reporter antibody from the signal of step 3, and
Step 5: determining the presence and/or amount of norovirus-reactive antibodies in the sample from the presence and/or amount of the reporter antibody determined in step 4.

The present disclosure is therefore further directed to a method for determining the presence and/or amount of norovirus-reactive antibodies in a sample from a subject, wherein the method comprises the steps of:
Step 1: providing a kit, including an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies as described above,
wherein the first reporter antibody binds to the first norovirus VLP and does not bind to the second norovirus virus like particle, and
wherein the second reporter antibody binds to the second norovirus VLP and does not bind to the first norovirus VLP;
Step 2: contacting the amount of the at least two microsphere complexes with the sample to allow binding of the norovirus-reactive antibodies in the sample to the first and/or the second norovirus VLPs while competing with the at least two reporter antibodies;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the reporter antibody bound to the norovirus VLPs of the at least one microsphere in step 2 upon irradiation with a second light source;
Step 4: repeating step 3 until at least 30 microspheres coupled to the same norovirus VLP are identified; and
Step 5: summarizing the detected signal from the reporter antibody in step 3 for all identified microspheres coupled to the same norovirus VLP, wherein the summarized signal is indicative for the presence and/or amount of norovirus-reactive antibodies in the sample, and wherein the method optionally comprises the further steps of:
Step 6: determining the presence and/or amount of the reporter antibody from the summarized signal of step 5, and
Step 7: determining the presence and/or amount of norovirus-reactive antibodies in the sample from the presence and/or amount of the reporter antibody determined in step 6.

### Abbreviations and Definitions Abbreviations

“PE” stands for phycoerythrin. “VLP” refers to virus like particle. “VLPs” refers to virus like particles. “MFI” refers to median fluorescent intensity. “Ab” and “Abs” stand for antibody and antibodies. “Ig” stands for immunoglobulin. “mAb” stands for monoclonal antibody. “CDR” stands for complementary determining region. “ELISA” refers to enzyme linked immunosorbent assay. “PGM” refers to pig gastric mucin. “GI”, “GII”, “GIV” refer to genogroup I, II, and IV, respectively. “VP1” stands for major viral capsid protein. “VP2” stand for minor viral capsid protein.

### Definitions

As used in the present disclosure and claims, the singular forms “a”, “an”, and “the” are to be construed to cover both the singular and the plural forms unless the context clearly dictates otherwise.

The term “and/or” as used in a phrase such as “A and/or B” herein is intended to include “A and B”, “A”, and “B”.

Open terms such as “include”, “including”, “contain”, “containing”, and the like mean “comprising”. These open-ended transitional phrases are used to introduce an open-ended list of elements, method steps, or the like that does not exclude additional, unrecited elements or method steps.

Unless otherwise indicated, all numbers expressing quantities of ingredients, properties such as molecular weight, reaction conditions, and so forth used in the specification and claims are to be understood as being modified in all instances by the term “about”. As used herein the terms “about” and “approximately” means within 10 to 15% of the number. In some embodiments, “about” means within 5 to 10% of the number.

### Antibody and Related Terms

As used herein, the term “antibody (Ab)” (plural: “antibodies (Abs)”) refers to an immunoglobulin (Ig) molecule, generally comprised of four polypeptide chains, two heavy (H) chains and two light (L) chains interconnected by disulfide bonds (full length Ab) and includes any naturally occurring, enzymatically obtainable, synthetic, or genetically engineered polypeptide or glycoprotein that specifically binds an antigen to form a complex. Abs can be obtained using standard recombinant DNA techniques. In a full length Ab, each heavy chain is comprised of a heavy chain variable region (VH) and a heavy chain constant region (CH). The heavy chain constant region is comprised of three domains, CH1, CH2 and CH3. Each light chain is comprised of a light chain variable region (VL) and a light chain constant region (CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In certain embodiments of the present disclosure, the FRs of the Ab may be identical to the human germline sequences, or may be naturally or artificially modified. The terms Ab or Abs may also refer to any functional fragment, mutant, variant, or derivative thereof. Such functional fragment, mutant, variant, or derivative antibody formats are known in the art. Ab fragments such as Fab or F(ab′)2 fragments, can be prepared from full length Abs using conventional techniques such as papain or pepsin digestion, respectively, of full length Abs. Functional fragments are in particular (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1), which comprises a single variable domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In some embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such functional fragments are known in the art (Kontermann and Dubel eds., Antibody Engineering (2001) Springer-Verlag. New York. 790 pp. (ISBN 3-540-41354-5)). The Ab may be described by the term “anti-antigen Ab” to express to which antigen the Ab is able to bind. For instance, an “anti-norovirus Ab” refers to an Ab that binds to a norovirus antigen. Ab or Abs may be mono-specific, bi-specific, or multi-specific. Multi-specific Abs may specifically bind different epitopes of one antigen or may specifically bind two or more unrelated antigens. See, e.g., Tutt et al., 1991, J. Immunol. 147:60-69; Kufer et al., 2004, Trends Biotechnol. 22:238-244. Abs including any of the multi-specific antigen-binding molecules of the present disclosure, or variants thereof, may be constructed using standard molecular biological techniques (e.g., recombinant DNA and protein expression technology), as will be known to a person of ordinary skill in the art, for instance intracellular expression systems. Abs may be multivalent Abs comprising two or more antigen binding sites. Substitution of one or more CDR residues or omission of one or more CDRs is also possible. Abs have been described in the scientific literature where one or two CDRs can be dispensed with barely an effect for binding. Analysis of the contact regions between Abs and their antigens, based on published crystal structures, revealed that only about one fifth to one third of CDR residues actually contact the antigen. Moreover, many Abs have one or two CDRs were no amino acids are in contact with an antigen (Padlan et al. FASEB J. 1995, 9: 133-139, Vajdos et al., J Mol Biol 2002, 320:415-428). CDR residues not contacting antigen can be identified based on previous studies (for example residues H60-H65 in CDR2 of the heavy chain are often not required), from regions of Kabat CDRs lying outside Chothia CDRs, by molecular modeling and/or empirically. If a CDR or residue(s) thereof is omitted, it is usually substituted with an amino acid occupying the corresponding position in another human Ab sequence or a consensus of such sequences. Positions for substitution within CDRs and amino acids to substitute can also be selected empirically. Empirical substitutions can be conservative or non-conservative substitutions. The terms Ab or Abs may refer to Ab or Abs that originate from certain origin species that for example include rabbit, mouse, human, monkey, or rat (rabbit Ab, mouse Ab, human Ab, monkey Ab, or rat Ab). For instance, rabbit origin may be intended to include Abs having variable and constant regions derived from rabbit germline immunoglobulin sequences. Abs may comprise one or more amino acid substitutions, insertions, and/or deletions as compared to corresponding germline sequences. The Abs may also include amino acid residues not encoded by the origin species germline immunoglobulin sequences (e.g. mutations introduced by random or site-specific mutagenesis in vitro or in vivo), for example in the CDRs. As used herein, an Ab or Abs originating from a certain origin species (e.g. rabbit) may also refer to an Ab or Abs in which CDR or other sequences derived from the germline of another mammalian species (e.g. mouse) have been grafted onto the origin species (e.g. rabbit) framework region (FR) sequences. Abs may be chimeric Abs. Chimeric Abs may encompass sequences derived from the germline of different species and may also include further amino acid substitutions or insertions. Abs may be humanized Abs that are human immunoglobulins that contain minimal non-human (e.g., murine) sequences. Typically, in humanized antibodies residues from the human CDR are replaced by residues from the CDR of a non-human species (e.g., mouse, rat, rabbit, and hamster, etc.; Jones et al., Nature 1986; 321:522-525; Riechmann et al., Nature 1988, 332:323-327; Verhoeyen et al., Science 1988, 239:1534-153). Non-limiting examples of methods used to generate humanized antibodies are described in U.S. Pat. No. 5,225,539; Roguska et al., Proc. Natl. Acad. Sci. 1994, USA 91:969-973; and Roguska et al., Protein Eng. 1996; 9:895-904. Abs can be of any class or isotype (e.g., IgG, IgE, IgM, IgD, IgA and IgY) and subclass (e.g., IgG1, IgG2, IgG3, IgG4, IgA1 and IgA2). In some embodiments, the antibody is of the IgG isotype. In some embodiments, the Ab is of the IgM isotype. In some embodiments, the Ab is of the IgA isotype. The different classes of immunoglobulins have different and well-known subunit structures and three-dimensional configurations. Abs may comprise sequences from more than one class or subclass. The terms Ab or Abs may refer to a neutralizing or non-neutralizing Ab or neutralizing or non-neutralizing Abs. The terms Ab or Abs may refer to a monoclonal Ab or monoclonal Abs. The terms Ab or Abs may refer to a reporter Ab or reporter Abs. The terms Ab or Abs may refer to a detection Ab or detection Abs.

As used herein, the term “complementary determining region (CDR)” refers to the CDR within the Ab variable sequences. There are three CDRs in each of the variable regions of the heavy chain (VH) and the light chain (VL), which are designated CDR1, CDR2 and CDR3 (or specifically VH-CDR1, VH-CDR2, VH-CDR3, VL-CDR1, VL-CDR2, and VL-CDR3), for each of the variable regions. The term CDR may refer to a group of three CDRs that occur in a single variable region capable of binding the antigen. The exact boundaries of these CDRs can be defined differently according to different systems. The system described by Kabat (Kabat et al., Sequences of Proteins of Immunological Interest (National Institutes of Health, Bethesda, Md. (1987) and (1991)) refers to an unambiguous residue system applicable to any variable region of an antibody, but also provides precise residue boundaries defining the three CDRs. These CDRs may be referred to as Kabat CDRs. For the VH region, the hypervariable region ranges from amino acid positions 31 to 35 for VH-CDR1, amino acid positions 50 to 65 for VH-CDR2, and amino acid positions 95 to 102 for VH-CDR3. For the VL region, the hypervariable region ranges from amino acid positions 24 to 34 for VL-CDR1, amino acid positions 50 to 56 for VL-CDR2, and amino acid positions 89 to 97 for VL-CDR3. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. In some embodiments, CDRs are defined according to Kabat or Chothia methods.

As used herein, the term “framework”, “framework region (FR)” or “framework sequence” refers to the remaining sequences of a variable region minus the CDRs. Because the exact definition of a CDR sequence can be determined by different systems, the meaning of a framework sequence is subject to correspondingly different interpretations. The six CDRs (VL-CDR1, VL-CDR2, and VL-CDR3 and VH-CDR1, VH-CDR2, and VH-CDR3) also divide the framework regions on the light chain (L) and the heavy chain (H) into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “constant region” of an Ab refers to the heavy chain constant region (CH) and/or the light chain constant region (CL).

As used herein, the term “variable region” of an Ab refers to the heavy chain variable region (VH) and/or the light chain variable region (VL).

As used herein, the terms “binds to”, “is binding to”, or “capable of binding to” refer within the context of an Ab that binds to or is binding to or is capable of binding to, to an Ab that is able to bind a certain antigen. The antigen can itself be an antibody. Ability of binding to a certain antigen can be investigated by methods well known in the art including ELISA, or bio-layer interferometry (BLI). In some embodiments, an Ab that binds to or is binding to or is capable of binding to an antigen provides a signal when tested for binding to the antigen in suitable methods which is at least 10%, at least 25%, at least 35%, at least 50%, at least 60%, at least 75%, at least 85%, at least 90%, at least 95%, or 100% higher than the background signal. In an embodiment, the Ab is able to bind to the antigen with the Ab variable region.

As used herein, the term “does not bind to” refers within the context of an Ab that does not bind to a certain antigen, to an Ab that is not able to bind a certain antigen. That an Ab “does not bind” to a certain antigen can be determined in methods well known in the art including ELISA, or bio-layer interferometry (BLI). In some embodiments, an Ab that does not bind to a certain antigen provides a signal when tested for binding to the antigen in suitable methods which is below the background signal or only slightly above the background signal.

As used herein, the term “allow binding” refers within the context of an Ab to a situation, wherein an Ab is incubated with an antigen like a norovirus VLP coupled to a microsphere or another Ab such as a reporter Ab for a certain time to enable the Ab to bind to the antigen. If an Ab does not bind to the antigen, no binding will occur.

As used herein, the term “bound to” refers within the context of an Ab that is bound to, to an Ab that is bound to an antigen. The Ab is bound to the antigen with the antibody variable region. The antigen within that context may be a norovirus VLP or another antibody. For instance, a detection Ab is bound to reporter Ab with the detection Ab variable region.

As used herein, a “norovirus-reactive antibody” is an Ab that is capable of binding to a norovirus antigen. A norovirus-reactive antibody may also be a norovirus-neutralizing and/or norovirus-blocking Ab. A norovirus-reactive antibody may be an Ab that only binds to one norovirus or an Ab capable of binding to different noroviruses, i.e. a cross-reactive Ab. For instance, a norovirus-reactive antibody may be capable of binding to a norovirus antigen from a norovirus of genogroup I and a norovirus antigen from a norovirus of genogroup II.

A “norovirus-neutralizing Ab”, as used herein, is intended to refer to an Ab which provides a signal above the lower limit of detection and/or the background in a norovirus neutralization assay. In some embodiments, the signal is at least 10% or at least 20% or at least 30% or at least 50% or at least 60% or at least 80% or at least 90% above the lower limit of detection and/or the background in a norovirus neutralization assay. A norovirus-neutralizing Ab may be used alone or in combination as prophylactic or therapeutic agent with other anti-viral agents upon appropriate formulation, or in association with active vaccination, or as a diagnostic tool. The term “norovirus-neutralizing Ab” may refer to an Ab which prevents, inhibits, reduces, impedes, or interferes with the ability of a norovirus to initiate and/or perpetuate an infection in a host. The epitope to which a norovirus-neutralizing Ab binds to may be referred to as a “neutralizing epitope”. A norovirus-neutralizing Ab may also be a norovirus-blocking Ab.

A “norovirus neutralization assay”, as used herein, refers to any assay that measures norovirus neutralizing Abs. For instance, one norovirus neutralizing assay uses human intestinal enteroids (HIEs). This assay detects neutralizing Abs within a sample by incubation of a live norovirus with the sample and monitoring infection of the organoids by the live norovirus. This method is described for instance in Ettayebi et al., Science 2016, 353, 1387-1393; Jones et al., Science 2014, 346, 755-759.

A “norovirus-blocking Ab”, as used herein, is intended to refer to an Ab which provides a signal above the lower limit of detection and/or the background in a norovirus blockade assay. In some embodiments, the signal is at least 10% or at least 20% or at least 30% or at least 50% or at least 60% or at least 80% or at least 90% above the lower limit of detection and/or the background in a norovirus blockade assay. A norovirus-blocking Ab blocks the binding of norovirus VLPs and/or noroviruses to cell surface carbohydrates, such as HBGAs. A norovirus-blocking Ab may also be a norovirus-neutralizing Ab.

A “norovirus blockade assay”, as used herein, refers to an assay that uses cell surface carbohydrates, such as HBGAs. The assay detects norovirus-blocking Abs within a sample by incubation of norovirus VLPs with the sample and monitoring binding of said norovirus VLPs to the cell surface carbohydrates. A commonly used substrate containing cell surface carbohydrates is PGM (“PGM blockade assay”). The method is described for instance in Haynes et al., Viruses 2019, 11, 392, doi:10.3390/v11050392).

As used herein, the term “antibody titer” refers to a certain amount of Ab within a sample. The sample may be a blood plasma, urine, blood, or serum sample. An antibody titer can be expressed as the highest dilution (in a serial dilution row) that still gives a positive test result or that gives half maximal signal in a test. An antibody titer may also be expressed in the form of an interpolation titer, wherein values indicative for the presence of an Ab in a sample are interpolated at a certain value resulting from a control, such as at the 50% maximum value received with the control. Consequently, the term “blocking-antibody titer” or “blocking titer” refers to a certain amount of norovirus-blocking Abs within a sample. An Ab titer can be determined by various methods known in the art including enzyme linked immunosorbent assay (ELISA), norovirus blockade assay (resulting in “blocking titers”), or norovirus neutralization assay (resulting in “neutralizing titers”).

As used herein, the term “enzyme linked immunosorbent assay (ELISA)” refers to an immunoassay for the measurement of Abs or antigens depending on the ELISA set-up. A key feature of all ELISA set-ups is the application of a plate on which Abs or antigens are immobilized. For instance, in order to determine Abs within a sample, a corresponding antigen to which the Abs bind to is immobilized on the plate. In another set-up, Abs are immobilized on the plate to detect antigens within a sample. The signal of an ELISA is generated by an enzymatic reaction, producing a signal that can be, for instance, detected by spectrophotometric methods. A common example of an enzyme applied is horseradish peroxidase. Common ELISA set-ups include direct ELISA, sandwich ELISA, competitive ELISA, and reverse ELISA.

As used herein, the term “monoclonal Ab” (“mAb”) refers to an Ab obtained from a population of substantially homogenous Abs that bind to the same antigenic determinants (epitopes). “Substantially homogeneous” means that the individual Abs are identical except for possibly naturally occurring mutations that may be present in minor amounts. This is in contrast to polyclonal antibodies that typically include different antibodies directed against various, different antigenic determinants (epitopes). A monoclonal Ab may be generated by hybridoma technology according to methods known in the art (Kohler and Milstein, Nature 1975, 256:495-497), phage selection, recombinant expression, and transgenic animals.

As used herein, the term “polyclonal Ab” refers to an Ab obtained from a sample of an immunized animal e.g. mouse or rabbit serum. A characteristic of a mixture of polyclonal Abs is that the Abs do not all bind to the same epitope.

As used herein, the term “detection Ab” refers to an Ab that is applied in the methods of the present disclosure, as well as to an Ab that is part of the kits of the present disclosure. In some embodiments, the detection Ab is capable of binding to norovirus-reactive antibodies within a sample with the variable region of the detection antibody. The detection Ab, independent of its specificity for an antigen, is attached to a detectable label. In some embodiments, the detection Ab is attached to a detectable label via the heavy chain constant region of the detection antibody. In some embodiments, the detectable label is a fluorescence label such as PE.

As used herein, the term “reporter Ab” refers to an Ab that is applied in the methods of the present application, as well as to an Ab that is part of the kits of the present disclosure. The reporter Ab is capable of binding to one or more norovirus VLPs with the variable region of the reporter antibody. The reporter Ab is capable of competing with other Abs, present for instance within a sample, for binding to the one or more norovirus VLPs. In some embodiments, the reporter Ab is directly attached to a detectable label. In some embodiments, the reporter Ab is directly attached to a detectable label via the heavy chain constant region of the detection antibody. In these embodiments, the reporter Ab is applied in the methods of the present disclosure without the additional use of a secondary reporter Ab, as the reporter Ab can itself be detected by the detectable label. In other embodiments, the reporter Ab is indirectly attached to a detectable label. In these other embodiments, the reporter Ab is applied in the methods of the present disclosure together with a secondary reporter Ab. The reporter Ab may be a cross-reactive antibody. In some embodiments, the reporter Ab is a mAb and/or a norovirus-neutralizing Ab and/or a norovirus-blocking Ab. The reporter antibody shows a certain EC₅₀ value towards the one or more norovirus VLPs, such as, for instance, 2 μg/mL or lower, 1 μg/mL or lower, 0.5 μg/mL or lower, 0.1 μg/mL or lower, 0.05 μg/mL or lower, or 0.01 μg/mL or lower.

As used herein, the term “secondary reporter Ab” refers to an antibody that is capable of binding to a reporter antibody. In some embodiments, the secondary reporter Ab binds to the heavy chain constant region of the reporter antibody with the variable region of the secondary reporter Ab. Within the meaning of the disclosure, the secondary reporter Ab is directly attached to a detectable label. In some embodiments, the detectable label is attached to the heavy chain constant region of the secondary reporter antibody.

As used herein, a “cross-reactive antibody” refers to an Ab that is capable of binding to more than one antigen. For instance, an Ab may be referred to as “cross-reactive antibody”, if the Ab is binding to a GII.4 norovirus or norovirus VLP and is also binding to a GII.1 norovirus or norovirus VLP. For instance, an antibody that is cross-reactive to one or more norovirus antigens shows an EC₅₀ value towards the one or more norovirus antigens of 2 μg/mL or lower, 1 μg/mL or lower, 0.5 μg/mL or lower, 0.1 μg/mL or lower, 0.05 μg/mL or lower, or 0.01 μg/mL or lower.

As used herein, the term “EC₅₀ value” refers to the concentration of an Ab, such as a reporter Ab, required to achieve 50% maximal binding at saturation to a norovirus VLP to which a microsphere is coupled to. The EC₅₀ value is a measure for the affinity of an Ab towards the norovirus VLP. The smaller the EC₅₀ value the higher the affinity.

The term “competing” or “competes with”, as used herein, refers to a situation in which a first Ab competes with a second Ab for a binding site on an antigen (i.e. a norovirus VLP). The term includes situations in which the Abs are applied concomitantly to the antigen or one after another. One of the two Abs may be a reporter Ab and the other of the two Abs may be present within a sample. Specifically, in a first orientation, the first Ab is allowed to bind to a norovirus VLP followed by assessment of binding of the second Ab to the norovirus VLP. In a second orientation, the second Ab is allowed to bind to a norovirus VLP followed by assessment of binding of the first Ab to the norovirus VLP. In a third orientation, the first and the second Ab are concomitantly allowed to bind to a norovirus VLP. The Abs may be allowed to bind under saturating conditions. As will be appreciated by a person of ordinary skill in the art, the first Ab that competes for binding with the second Ab may not necessarily bind to the same epitope as the second Ab, but may sterically block binding of the second Ab by binding an overlapping or adjacent epitope. Two Abs bind to the same or overlapping epitope if each competitively inhibits (blocks) binding of the other to the antigen. Alternatively, two Abs have the same epitope if essentially all amino acid mutations in the antigen that reduce or eliminate binding of one Ab reduce or eliminate binding of the other. Two Abs have overlapping epitopes if some amino acid mutations that reduce or eliminate binding of one antibody reduce or eliminate binding of the other. The capability of one Ab to inhibit (block) binding of an antigen by another Ab is a measure of the ratio of the affinities of the two Abs for the antigen. If one Ab strongly inhibits (blocks) binding of another Ab to an antigen, the affinity of the one Ab for the antigen is higher than the affinity of the other Ab for the antigen. For instance, a reporter Ab that shows an EC₅₀ value towards a norovirus VLP of 0.05 μg/mL (which is a measure for affinity of the reporter Ab for the norovirus VLP) will strongly inhibit (block) binding of another anti-norovirus Ab that sows an EC₅₀ value towards the norovirus VLP of 1 μg/mL if the Abs bind to the same or overlapping epitopes.

### Detection System and Label

The term “detectable label”, as used herein, refers to any compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

As used herein, the term “antibody is attached to a detectable label”, refers to an Ab that is connected to a detectable label. The connection can be a covalent connection, which occurs for instance upon formation of an amide bound between the antibody and the detectable label. The type of connection is dependent on the functional groups available on the Ab and on the detectable label. In some embodiments, the antibody is attached to the detectable label with the heavy chain constant region of the antibody.

As used herein, the term “detection system” refers to any system which is suitable for determining values indicative for the presence and/or amount of a detection or a reporter antibody. The detection system may also be able to determine values indicative for the presence and/or amount of a microsphere. The microsphere may be by individually identified by a detectable label. The detection system comprises one or more light sources.

As used herein, the term “light source” refers to any light source that is suitable to irradiate and thereby excite a detectable label as for instance a fluorescence dye. In some embodiments the light source may be a laser. In some embodiments, the light source may be a light emitting diode (LED).

### Microsphere and Immunoassay

As used herein, the terms “microsphere” or “microspheres” refer to small particles to which molecules like antigens (i.e. VLPs) can be attached to for use in the methods of the present disclosure. The terms microsphere, microparticle, bead, or microbead can be used interchangeably and bear equivalent meanings. A microsphere may be identified by a detectable label.

As used herein, the term “microsphere complex” refers to a complex of microsphere and antigen. The antigen may be covalently attached to the microsphere. The antigen may be a VLP e.g. a norovirus VLP. The antigen may be attached to the microsphere by carbodiimide coupling using 1-Ethyl-3-(3-dimethylaminopropyl)-carbodiimide (EDC) and N-Hydroxysulfosuccinimide (Sulfo-NHS).

As used herein, the term “immunoassay” refers to an assay that detects, determines, identifies, characterizes, quantifies, or otherwise measures the presence and/or amount of a molecule through the use of an Ab or antigen. The molecule detected by the immunoassay can be present in biological samples (e.g. serum or blood plasma). The molecule detected by the immunoassay may be itself an Ab or antigen. The immunoassay may include, for example, direct or competitive binding assays using techniques such as ELISA, immunoprecipitation assays, or microsphere immunoassays.

As used herein, the term “microsphere immunoassay” refers to an assay that detects, determines, identifies, characterizes, quantifies, or otherwise measures the presence and/or amount of Abs with the use of microspheres coupled to an antigen to which the Abs are able to bind. The Abs detected by the microsphere immunoassay can be present in biological samples (e.g. serum or blood plasma).

The term “competitive microsphere immunoassay” also shortly “competitive assay set-up” refers to a microsphere immunoassay that detects, determines, identifies, characterizes, quantifies, or otherwise measures the presence and/or amount of Abs by using microspheres coupled to an antigen to which the Abs are able to bind and a reporter Ab. In a competitive microsphere immunoassay the Abs, which may be present, for instance, within a sample and the reporter Ab are competing for binding to the antigen. The presence and/or amount of reporter Ab bound to the antigen is thus indicative for the presence and/or amount of the Abs, which are capable of competing with the applied reporter Ab. Thus, a competitive microsphere immunoassay can be used for determining “specific” Abs or antibody titers, wherein “specific” in that context means that the detected Abs in the sample are capable of competing with the reporter antibody for antigen binding. Thereby, a “specific” immune response or immune status may be determined, wherein “specific” in that context means that the immune response or immune status is characterized by the determined “specific” Abs capable of competing with the applied reporter Ab for antigen binding.

The term “non-competitive microsphere immunoassay” also shortly “non-competitive assay set-up” refers to a microsphere immunoassay that detects, determines, identifies, characterizes, quantifies, or otherwise measures the presence and/or amount of Abs by using microspheres coupled to an antigen to which the Abs are able to bind and a detection Ab that binds to the heavy chain constant region of the Abs. Thereby, the Abs, which may be present, for instance, within a sample, are incubated with the microspheres in order to allow binding of the Abs to the antigen. Thereafter, the detection Ab is added in order to detect, determine, identify, characterize, quantify, or otherwise measure the presence and/or amount of the Abs. Thus, a non-competitive microsphere immunoassay can be used for determining “total” antibody titers or antibody amounts as this assay detects essentially all or a major part of Abs capable of binding to the antigen to which the microsphere is coupled to. Thereby, a “complete” immune response may be determined, wherein “complete” within that context means that the determined immune response is characterized by the determined “total” antibody titers or antibody amounts.

### Virus Like Particle and Norovirus

As used herein, the term “antigen” refers to any substance which can be bound by an Ab. Antigens may induce an immune response within a subject. An antigen may have one or more epitopes. An antigen may be a protein, polypeptide, carbohydrate, polynucleotide, lipid, or combinations thereof. As used herein, antigen may refer to a norovirus VLP or a part of a norovirus VLP.

As used herein, the term “epitope” or “antigenic determinant” refers to the part of an antigen that interacts with a specific antigen-binding site in the variable region of an Ab molecule known as a paratope. A single antigen may have more than one epitope. Thus, different Abs may bind to different areas on an antigen and may have different biological effects. For example, the term “epitope” also refers to a site on an antigen to which B and/or T cells respond. Epitopes may be defined as structural or functional. Functional epitopes are generally a subset of the structural epitopes and have those residues that directly contribute to the affinity of the interaction. The epitope to which the antibodies bind may consist of a single contiguous sequence of 2 or more (e.g., 2, 3, 4, 5, 6, 7, 8, 9, 10, 11, 12, 13, 14, 15, 16, 17, 18, 19, 20 or more) amino acids located within an antigen i.e. a linear epitope for instance in a domain of a NV E protein. Epitopes may also be conformational, that is, composed of a plurality of non-contiguous amino acids, i.e., non-linear amino acid sequence. A conformational epitope typically includes at least 3 amino acids, and more commonly, at least 5 amino acids, e.g., 7-10 amino acids in a unique spatial conformation. In some embodiments, epitopes may include determinants that are chemically active surface groupings of molecules such as amino acids, sugar side chains, phosphoryl groups, or sulfonyl groups, and, In some embodiments, may have specific charge characteristics. Epitopes formed from contiguous amino acids are typically retained on exposure to denaturing solvents, whereas epitopes formed by tertiary folding are typically lost on treatment with denaturing solvents. Various techniques known to persons of ordinary skill in the art can be used to determine whether an antibody interacts with one or more amino acids within a polypeptide or protein. Exemplary techniques include, for example, site-directed mutagenesis (e.g., alanine scanning mutational analysis). Other methods include routine cross-blocking assays (such as that described in Antibodies, Harlow and Lane, Cold Spring Harbor Press, Cold Spring Harbor, NY), peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267: 252-259; Engen and Smith (2001) Anal. Chem. 73: 256A-265A. Modification-Assisted Profiling (MAP), also known as Antigen Structure-based Antibody Profiling (ASAP) may be used to sort Abs binding the same antigen into groups of Abs binding different epitopes. MAP is a method that categorizes large numbers of Abs directed against the same antigen according to the similarities of the binding profile of each antibody to chemically or enzymatically modified antigen surfaces (see US 2004/0101920). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. Abs may also be categorized according to their epitopes using Biolayer Interferometry (BLI).

As used herein, the terms “virus like particle (VLP)” or “virus like particles (VLPs)” refer to molecules that closely resemble viruses, but are non-infectious because they do not contain viral genetic material. VLPs can be prepared recombinant through the expression of viral structural proteins, which can then self-assemble into the VLPs. Suitable expression systems include eukaryotic expression systems like mammalian or insect expression systems.

As used herein, the term “norovirus VLP” refers to a VLP comprising at least one of the structural proteins (VP1, VP2) of one or more noroviruses. The structural proteins may be at least 80%, or at least 85%, or at least 90%, or at least 95% or 100% identical to amino acid sequences representing the structural proteins of the corresponding noroviruses. Throughout the application and the claims, specific norovirus VLPs are referred to as designated in Table 1. For instance, a GI.1 VLP refers to a norovirus VLP derived from the Hu/GI.1/Norwalk/1968/US norovirus with a VP1 sequence as shown in GenBank: AAB50466.2 and SEQ ID NO: 1, wherein the VP1 protein of the norovirus VLP may be at least 80%, or at least 85%, or at least 90%, or at least 95% or 100% identical to SEQ ID NO: 1. “Derived” within that context means that the VP1 sequence of the corresponding norovirus or a sequence that may be at least 80%, or at least 85%, or at least 90%, or at least 95% or 100% identical to the VP1 sequence is expressed in a suitable expression system in order to generate the VLP upon self-assembly.

As used herein, the term “norovirus” or “noroviruses” refer to members of the species of norovirus belonging to the family Caliciviridae. Noroviruses can cause acute gastroenteritis in humans. Noroviruses are currently classified phylogenetically into 7 different genogroups (GI-GVII) and more than 40 different genotypes. Viruses of GI, GII, and GIV infect humans, wherein viruses from GI and GII account for nearly all human infections. Since the mid-1990s, viruses from genogroup II genotype 4 (GII.4) have caused the majority of outbreaks. Noroviruses from genogroup II genotype 4 (GII.4) have been further classified into variants. Non-limiting examples of norovirus strains include Norwalk virus (NV, GenBank M87661), Southampton virus (SHV, GenBank L07418), Desert Shield virus (DSV, U04469), Hesse virus (HSV), Chiba virus (CHV, GenBank AB042808), Hawaii virus (HV, GenBank U07611), Snow Mountain virus (SMV, GenBank U70059), Toronto virus (TV, Leite et al., Arch. Virol. 141:865-875), Bristol virus (BV), Jena virus (JV, AJ01099), Maryland virus (MV, AY032605), Seto virus (SV, GenBankAB031013), Camberwell (CV, AF145896), Lordsdale virus (LV, GenBank X86557), Grimsby virus (GrV, AJ004864), Mexico virus (MXV, GenBank U22498), Boxer (AF538679), C59 (AF435807), VA115 (AY038598), BUDS (AY660568), Houston virus (HoV), Minerva strain (EF 1269631), Laurens strain (EF 1269661), MOH (AF397156), Parris Island (PiV, AY652979), VA387 (AY038600), VA207 (AY038599), and Operation Iraqi Freedom (OIF, AY675554). Further examples include Hu/GI.1/Norwalk/1968/US (GenBank M87661), Hu/GI.2/Jingzhou/2013401/CHN (GenBank KF306212), Hu/GI.3/JKPG_883/SWE/2007 (GenBank FJ711164.1), Hu/GI.4/1643/2008/US (GenBank GQ413970), Hu/GI.5/Siklos-HUN5407/2013/HUN (Gen Bank KJ402295), Hu/GI.6/TCH-099/USA/2003 (GenBank KC998959), Hu/GI.7/Providence191/2010/USA (GenBank JN899243), Hu/GII.3/NIHIC8.1/2011/USA (GenBank KC597140), Hu/GII.4/Houston/TCH186/2002/US (GenBank JX459908), Hu/GII.4/DenHaag89/2006/NL (GenBank EF126965.1), Hu/GII.4/Yerseke38/2006/NL (GenBank EF126963.1), Hu/GII.4/Sydney/NSW0514/2012/AU (GenBank JX459908), Hu/GII.4/031693/USA/2003 (GenBank JQ965810.1), Hu/GII.6/Ehime120246/2012/JP (GenBank AB818400), Hu/GII.12 strain E5152 (GenBank, Hu/GII.17/C142/GF/1978 (GenBank JN699043), Hu/GII.17/JP/2014/Nagano7-1 (GenBank LC043139), and Hu/GII.17/HKG/2015/CUHK-NS-513 (GenBank KP698931.1). Further examples of noroviruses are listed in WO 2020/017542, Table 1 or in the section “microsphere complex” below. Throughout the disclosure, the term “norovirus” may be used to refer to any norovirus strain of any genogroup or genotype or variant. The term “norovirus” may also refer to norovirus consensus sequences from, for instance, two or more noroviruses such as GII.4 variants. Construction of a norovirus consensus sequence is for instance shown in WO 2010/0175242 and Parra et al., Vaccine 2012, 30(24):350-3586. Within the meaning of the disclosure a “norovirus strain” may be used to refer to any norovirus of any genogroup or genotype or variant.

As used herein, the term “genogroup” refers to a classification for noroviruses by phylogenetic clustering. Currently, noroviruses are classified into seven genogroups (GI-GVII). For references see for example Kroneman et al., Arch Virol 2013, 158:2059-2068; Preeti et al., Journal of General Virology 2019, 100:1393-1406.

As used herein, the term “genotype” refers to a further division of norovirus genogroups by phylogenetic clustering. Genogroups were divided into more than 40 genotypes. For instance, a norovirus of the genogroup II and genotype 4 will be designated as GII.4. For references see for example Kroneman et al., Arch Virol 2013, 158:2059-2068; Preeti et al., Journal of General Virology 2019, 100:1393-1406.

As used herein, the term “variant” refers to a further division of norovirus genotypes by phylogenetic clustering. Subtyping of GII.4 strains into variants is based on phylogenetic clustering. For references see for example Kroneman et al., Arch Virol 2013, 158:2059-2068; Preeti et al., Journal of General Virology 2019, 100:1393-1406.

As used herein, a “consensus sequence” is determined by aligning and comparing the viral amino acid sequences of two or more viruses. A consensus sequence may also be determined by aligning and comparing the nucleotide sequences of two or more viruses. The consensus sequence may be determined from nucleotide or amino acid sequences of two or more, three or more, four or more, five or more, six or more, seven or more, eight or more, nine or more circulating strains of a virus. The sequence alignment may result in one or more variable amino acids or nucleotides at a certain sequence position. In the consensus sequence one of the two or more amino acids or nucleotides at a variable position is selected. In some embodiments, the amino acid or nucleotide which is selected is the amino acid or nucleotide of a recently circulating strain, or a strain that is more commonly associated with disease or more commonly occurring amongst the strains being evaluated.

As used herein, the term “structural protein” refers to viral proteins that are structural components of the mature virus. Within the context of noroviruses, structural proteins include VP1 and VP2 proteins. The term “structural protein” may thus refer to both VP1 and VP2, or only to one of both.

### Sample and Subject

As used herein, the term “sample” refers to any sample derived from a subject. Within the meaning of this disclosure, the sample is present outside the subject body, i.e. is not obtained from the subject during the methods of the present disclosure. Consequently, diagnosing-methods are in vitro diagnosing-methods and methods for determining protection are in vitro methods for determining protection. The sample may be blood, urine, saliva, cerebrospinal fluid, and lymph fluid. In some embodiments, the sample is a serum or blood plasma sample. Within this disclosure, the term “plasma” refers to blood plasma. The sample may contain norovirus-reactive Abs. The sample may be pre-treated prior to use in the methods of the present disclosure. Methods for pre-treating can involve purification, filtration, distillation, concentration, inactivation of interfering compounds, and the addition of reagents. In some embodiments the sample is heat-inactivated.

As used herein, the terms “subject” or “subjects” can include any individual. The subject may be a mammal. A mammal may be, but is not limited to, a mouse, a primate, a non-human primate, a human, a rabbit, a cat, a rat, a horse, a sheep. The subject may be a pregnant mammal, and in particular a pregnant woman. The subject may be a newborn up to 2 months of age or a child, which is 2 months to 5 years old. The subject may be a patient, for whom prophylaxis or therapy is desired. The subject may be norovirus naive or norovirus exposed. The subject may be from a norovirus endemic region or a norovirus non-endemic region. The subject may be from a norovirus non-endemic region travelling to a norovirus endemic region. The subject may be vaccinated with a norovirus vaccine. The subject may be an immune-suppressed person or a person above 70 years of age.

The term “norovirus naive” or “norovirus negative” as used herein refers to a subject that does not have Abs directed to norovirus above the detection limit as determined in a test measuring norovirus antibody titers such as blocking and/or neutralizing titers. A norovirus naive subject may have not been exposed to a norovirus and therefore does not carry Abs directed to the norovirus. A norovirus naïve subject may also be a subject that has once been exposed to a norovirus and that once had Abs directed to the norovirus, but the Abs directed to the norovirus disappeared over time.

As used herein, the term “norovirus exposed” or “norovirus positive” refers to a subject that does have Abs directed to norovirus above the detection limit as determined in a test measuring norovirus antibody titers such as blocking and/or neutralizing titers. Abs directed to norovirus can be the result of a norovirus infection or vaccination with a norovirus vaccine triggering the generation of Abs directed to norovirus by an immune response of the subject.

As used herein, an “immune response” refers to a subject's immune response to norovirus exposure. In particular, the immune response includes the formation of Abs to the norovirus. The term immune response may also include formation of neutralizing and/or blocking Abs to the norovirus. It may also include the stimulation of a cell-mediated response or the formation of Abs to structural proteins such as VP1 protein. It may also include the stimulation of a cell-mediated response.

As used herein, “endemic region” refers to a region where a disease or infectious agent is constantly present and/or usually prevalent in a population within this region. As used herein, “non-endemic region” refers to a region from which the disease is absent or in which it is usually not prevalent. Accordingly, a “norovirus endemic region” refers to geographic areas in which an infection with norovirus is constantly maintained at a baseline level. A “norovirus non-endemic region” is a geographic area in which an infection with norovirus is not constantly maintained at a baseline level. Accordingly, subjects “from a norovirus endemic region” or “from a norovirus non-endemic region” refer to subjects living in geographic areas as defined above. It is possible, that certain norovirus strains are circulating in one area and are absent in the other. Thus, a region may be non-endemic for one norovirus strain but endemic for the other.

As used herein, “vaccinated” refers to a subject that has been administered a vaccine, with the aim to prevent the subject from developing one or more symptoms of a disease.

As used herein, the term “vaccine” refers to a prophylactic material providing at least one antigen capable of introducing an immune response in a subject. A “norovirus vaccine” provides at least one norovirus antigen. In some embodiments, the norovirus antigen is a norovirus VLP. A norovirus vaccine is described for instance in Parra et al. (Vaccine 2012, 30(24): 3580-3586, doi: 10.1016/j.vaccine.2012.03.050) and WO 2010/017542.

### Infection and Diagnosis

The term “norovirus infection” as used herein, refers to the disease or condition which results from contact to a norovirus, which is usually spread by the fecal-oral route. Infection may occur through contaminated food or water or person-to-person contact. It may also occur through contact with contaminated surfaces or through air from the vomit of an infected person. Risk factors include unsanitary food preparation and sharing close quarters. Noroviruses cause gastroenteritis. Infection is characterized by non-bloody diarrhea, vomiting, and stomach pain. Fever or headaches may also occur. A norovirus infection may also not be accompanied by norovirus specific symptoms, in such a case the infection may be asymptomatic or inapparent. A norovirus infection may be acute or convalescent.

As used herein, the term “acute norovirus infection” refers to a norovirus infection that is characterized by rapid onset of disease, a relatively brief period of symptoms, and resolution within days. A rapid norovirus infection is usually accompanied by early production of infectious virions and elimination of infection by the host immune system. Within an acute norovirus infection, Ab titers in body fluids are high compared to a convalescent virus infection.

As used herein, the term “convalescent norovirus infection” refers to a norovirus infection that has been eliminated by the host immune system. A characteristic of a convalescent norovirus infection is the existence of memory B-cells encoding for Abs against the norovirus that has caused the infection. Within a convalescent norovirus infection, Ab titers in body fluids are low compared to an acute norovirus infection.

As used herein, the term “diagnosing” refers to the application of methods that can be used to confirm or determine the likelihood of whether a patient is suffering from or had previously suffered from a given disease or condition i.e. a norovirus infection. Within the meaning of the disclosure, methods for diagnosing are in vitro methods for diagnosing.

As used herein, the term “established amounts of norovirus-reactive antibodies” refers to a certain amount of norovirus-reactive antibodies that are indicative for a norovirus infection. After norovirus infection, norovirus-reactive antibodies are produced in the subject and the amount of these norovirus-reactive antibodies thus enables to determine that the subject was infected with norovirus.

### Protection

As used herein, the term “protection against norovirus infection” refers to a condition wherein the amount of norovirus-reactive Abs within a subject is equal to or higher than protective amounts of norovirus-reactive Abs. As used herein, the term “protective amounts” refers to an amount of norovirus-reactive Abs associated with absence of disease caused by contact with the norovirus. The “protective amounts” may differ depending on the type of subject, e. g. protective amounts may be different from human to monkey and even different from children to adults. Protective amounts of Abs may be induced by administering a vaccine.

### BRIEF DESCRIPTION OF THE DRAWINGS

FIG. 1 Evaluation of coupling efficacy of GII.4/Sydney VLP to microspheres using eight different conditions (cf. Table 2). VLP-coupled microspheres were incubated with a polyclonal anti-GII.4/Sydney norovirus antibody and binding was detected using a suitable detection antibody directed against the polyclonal antibody. Median fluorescence intensity (MFI) is presented in dependency of the polyclonal antibody dilution (log (dilution factor)).

FIG. 2 Evaluation of IgM and IgA titers from subject #1 against 20 different norovirus VLPs using the 20-plex non-competitive assay set-up. Median fluorescence intensity (MFI) is presented in dependency of the serum sample dilution (log (dilution factor)).

FIG. 3 Evaluation of IgG titers from subject #1 (FIG. 3A) and IgM titers from subject #2 (FIG. 3B) against 20 different norovirus VLPs using the 20-plex non-competitive assay set-up. Median fluorescence intensity (MFI) is presented in dependency of the serum sample dilution (log (dilution factor)).

FIG. 4 Evaluation of IgA and IgG titers from subject #2 against 20 different norovirus VLPs using the 20-plex non-competitive assay set-up. Median fluorescence intensity (MFI) is presented in dependency of the serum sample dilution (log (dilution factor)).

FIG. 5 Epitope binning experiments using mouse mAbs resulted in 4 different clusters (I-IV). 10 mAbs were selected for further characterization (encircled).

FIG. 6 Epitope binning experiments using the 10 mouse mAbs selected and two human mAbs (1227, 1431). Some mAbs showed a slightly different grouping when compared to FIG. 5 as fewer antibodies were used for epitope binning in FIG. 6.

FIG. 7 Binding of mAbs to GII.4/Sydney VLPs in a microsphere immunoassay set-up. Incubation of rising mAb concentrations with GII.4/Sydney VLPs coupled to the microspheres. Median Fluorescent Intensity (MFI) is presented as a function of logarithmized mAb concentration.

FIG. 8 Singleplex competitive microsphere immunoassay using mAb 11F03 for analysis of different human serum samples. Incubation of serially dilution of human serum with GII.4/Sydney VLPs coupled to the microspheres. Median Fluorescent Intensity (MFI) is presented as a function of logarithmized serum dilution.

FIG. 9-11 Singleplex and duplex competitive microsphere immunoassay using anti-GI.1 Norwalk and anti-GII.4 Consensus mAbs for evaluating human serum samples BRH1434797 (FIG. 9, upper panel), BRH1434799 (FIG. 9, lower panel), BRH1434790 (FIG. 10, upper panel), BRH1434805 (FIG. 10, lower panel), and BRH1434806 (FIG. 11). Serial dilutions of the human serum samples were incubated with GI.1 Norwalk and/or GII.4 Consensus VLPs coupled to the microspheres. Median Fluorescent Intensity (MFI) is presented as a function of logarithmized serum dilution. NW:NW+CN refers to analysis of GI.1 Norwalk VLP-coupled microspheres present within a duplex set-up containing a mixture of GI.1 Norwalk and GII.4 Consensus VLP-coupled microspheres and anti-GI.1 Norwalk (17-1-1) and anti-GII.4 Consensus (4-1-3) mAbs. CN:NW+CN refers to analysis of GII.4 Consensus VLP-coupled microspheres present within a duplex set-up containing a mixture of GI.1 Norwalk and GII.4 Consensus VLP-coupled microspheres and anti-GI.1 Norwalk (17-1-1) and anti-GII.4 Consensus (4-1-3) mAbs. NW:CN, NW:NW, CN:NW, CN:CN refer to singleplex set-ups with GI.1 Norwalk VLP-coupled microspheres and anti-GII.4 Consensus (4-1-3) mAb, GI.1 Norwalk VLP-coupled microspheres and anti-GI.1 Norwalk (17-1-1) mAb, GII.4 Consensus VLP-coupled microspheres and anti-GI.1 Norwalk (17-1-1) mAb, and GII.4 Consensus VLP-coupled microspheres and anti-GII.4 Consensus (4-1-3) mAb, respectively.

FIG. 12 Duplex competitive microsphere immunoassay using anti-GI.1 Norwalk and anti-GII.4 Consensus mAbs for evaluating human serum samples. Incubation of serially dilutions of human serum with GI.1 Norwalk and GII.4 Consensus VLPs coupled to the microspheres. Median Fluorescent Intensity (MFI) is presented as a function of logarithmized serum dilution. The curves show MFI values resulting from analysis of GII.4 Consensus VLP-coupled microspheres. In addition, also a control, solely comprising mAb without serum was included (“monoclonal only”).

FIG. 13 Duplex competitive microsphere immunoassay using anti-GI.1 Norwalk and anti-GII.4 Consensus mAbs for evaluating human serum samples. Incubation of serially dilutions of human serum with GI.1 Norwalk and GII.4 Consensus VLPs coupled to the microspheres. Median Fluorescent Intensity (MFI) is presented as a function of logarithmized serum dilution. The curves show MFI values resulting from analysis of GI.1 Norwalk VLP-coupled microspheres. In addition, also a control, solely comprising mAb without serum was included (“monoclonal only”).

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. The specific embodiments mentioned within these sections can be combined as will be obvious to one skilled in the art.

### Microsphere Complex

The microsphere complex for use in the methods and kits of the present application comprises a microsphere coupled to a norovirus VLP. Specifications and embodiments within this section can be combined with any of the specifications and embodiments of the following sections.

### Microsphere

The microsphere useful for the present disclosure ranges in the size from about 0.01 to about 100 μm in diameter. In some embodiments, the microsphere ranges in size from about 1 to about 10 μm. In some embodiments, the microsphere ranges in size from about 5 to about 7 μm. In some embodiments, the microsphere has a diameter of about 6.5 μm. The size of a microsphere can be determined in practically any flow cytometry apparatus by so-called forward or small-angle scatter light.

The microsphere may be constructed of any material to which molecules like VLPs may be attached to. For example, acceptable materials for the construction of microspheres include but are not limited to: polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. In some embodiments of the present disclosure, microspheres are constructed of polystyrene.

The microsphere may comprise surface affinity groups for attachment of molecules. Said affinity groups may be, but are not limited to, Nia'⁰ (for immobilization of His-tagged molecules), Protein A, Protein G, Protein L, anti-human IgG Ab, anti-rabbit IgG Ab, anti-mouse IgG Ab, anti-goat IgG Ab, anti-FLAG Ab, streptavidin, avidin, and glutathione.

The microsphere may comprise functional groups on the surface useful for attachment of molecules, such as the norovirus VLPs of the present disclosure. Said functional groups may be, but are not limited to, carboxylates, esters, alcohols, carbamides, aldehydes, amines, sulfur oxides, nitrogen oxides, maleimides, or halides. In some embodiments, the microsphere comprises carboxylates on the surface. Molecules like norovirus VLPs can be covalently coupled to the microspheres using chemical techniques described herein. In some embodiments, molecules like norovirus VLPs can be coupled to the microsphere by carbodiimide coupling using 1-Ethyl-3-(3-dimethylaminopropyl)carbodiimide (EDC) and N-hydroxysulfosuccinimide (Sulfo-NHS). Thereby, the EDC is reacting to an unstable o-acylisourea ester with a carboxylate on the surface of the microspheres. The unstable o-acylisourea ester readily reacts with Sulfo-NHS to form a semi-stable amine reactive NHS-ester. The NHS-ester finally reacts with an amine group provided by an antigen, thereby forming a stable amide bond.

As amine-containing compounds other than those provided by the antigen, glycerol, urea, imidazole, azide, and some detergents may interfere with the carbodiimide coupling, they should be removed from the antigen preparation with a suitable buffer exchange method. For instance, a suitable buffer for carbodiimide coupling is 50 mM 2-(N-morpholino)ethanesulfonic acid (MES) buffer or phosphate buffer saline (PBS). The pH value for coupling may be between about 5 and about 9. Coupling of the antigen to the microsphere may be carried out by incubation for about 2 hours.

The microsphere may be magnetic. In some embodiments, the microsphere may be superparamagnetic. Magnetic microspheres can be easily captured by a magnetic plate separator for instance to wash the microspheres. A magnetic plate separator can be used for separating the microspheres within the 96-well plate from the solution within the wells of the 96-well plate by magnetic capture and refers to a construction for holding a 96-well plate. A magnetic plate separator enables the user to quickly decant the supernatant within the wells and washing of the wells, while fixing the microspheres at the bottom of the 96-well plate by magnetic capture. Application of a magnetic plate separator reduces the risk that microspheres are getting lost during washing procedures.

The microsphere may comprise a detectable label by which the microsphere can be identified with the help of a detection system. Identification of a microsphere likewise allows identification of the norovirus VLP, which is coupled to the microsphere.

Concerning the detection of such labels with suitable detection systems, reference is also made to the section “Detection system”.

In some embodiments, the detectable label is at least one fluorescent dye. The at least one fluorescent dye may be selected from the group consisting of squaraine, phthalocyanine, naphthalocyanine, and any derivative thereof. For instance, a derivative of a fluorescent dye may be the dye further comprising a methyl group at any position.

In some embodiments, the microsphere comprises one fluorescent dye. The microsphere can be identified by the emission signal of the one fluorescent dye upon irradiation with a suitable light source.

In other embodiments, the microsphere comprises one fluorescent dye in a different concentration than other microspheres, which comprise the same fluorescent dye. In such embodiments, the microsphere can be identified and distinguished from the other microspheres by the intensity of the emission signal of the one fluorescent dye upon irradiation with a suitable light source.

In some embodiments, the microsphere comprises two or more fluorescent dyes. The microsphere can be identified by the emission signal of the two or more fluorescent dyes upon irradiation with a suitable light source.

In some embodiments, the microsphere comprises the two or more fluorescent dyes in different concentrations (at a different ratio) than other microspheres, which comprise the same fluorescent dyes. In such embodiments, the microsphere can be identified and distinguished from the other microspheres by the intensity of the emission signal of the two or more fluorescent dyes upon irradiation with a suitable light source.

In some embodiments, where the microsphere comprises two or more fluorescent dyes, the emission signal of the two or more fluorescent dyes is resulting from an overlay of the emission signal of the single fluorescent dyes. The intensity of the emission signal is therefore indicative for the ratio of the two or more fluorescent dyes and therefor for the corresponding microsphere.

In one embodiment, one microsphere may comprise two fluorescent dyes having an emission signal maximum at 675 nm and another microsphere may comprise two different fluorescent dyes having an emission signal maximum at 700 nm.

The at least one fluorescent dye can be covalently attached onto the surface of the microsphere, or can be internally incorporated during microsphere production (i.e. polystyrene polymerization), or the microsphere can be dyed after production by placing the microsphere in a suitable solution comprising the at least one fluorescent dye. A suitable solution comprising the at least one fluorescent dye is for instance an organic solution.

The at least one fluorescent dye can be excited with any suitable light source as for instance a laser or a light emitting diode (LED) using a suitable detection system.

In some embodiments, different microspheres comprising different concentrations of fluorescent dyes can be excited by the same light source (e.g. the one or more fluorescent dyes at specific concentrations in the different microspheres are excitable by the same wavelength). In some embodiments, the different microspheres are excitable with a wavelength within the range from about 600 to about 650 nm. In some embodiments, the different microspheres are excitable with a wavelength of about 615 nm to about 640 nm. In some embodiments, the different microspheres are excitable with a wavelength of about 620 to about 635 nm. In some embodiments, the different microspheres are excitable with a wavelength of about 635 nm. An advantage of such a set-up is, that only one light source is needed for distinguishing all microspheres present within a microsphere mixture and thereby further simplifying a set-up in which multiple norovirus VLPs can be applied in one single experiment.

The microspheres may also be identified by their size, if different microspheres are of a different size using a suitable detection system. The size of the microspheres ranges from 0.01 to 100 μm in diameter. In some embodiments, the size of the microspheres ranges from about 1 to about 10 μm in diameter. For instance, one microsphere may be about 6 μm in diameter, another microsphere may be about 6.5 μm in diameter.

The microsphere may also be identified by a specific shape of the microsphere, if different microspheres are of a different shape using a suitable detection system.

To allow the simultaneous detection of antibodies reactive to different norovirus VLPs in one single experiment, microspheres with a different size or a different detectable label or a different shape are coupled to different norovirus VLPs and mixed. Microspheres coupled to the same norovirus VLP have the same size or the same detectable label or the same shape. Although the microspheres are mixed each microsphere can be identified by the specific size or detectable label or shape of the microsphere. Thereby, the norovirus VLP the microsphere is coupled to can be simultaneously identified.

Microspheres may be one out of the list consisting of MagPlex® microspheres, MicroPlex® microspheres, LumAvidin® microspheres, MagPlex®-Avidin microspheres, and SeroMAP® microspheres produced by the Luminex Corporation (Austin, Texas). The type of microsphere which can be used depends on the detection system applied (reference is also made to the section “Detection system”).

In some embodiments, the microspheres are the MagPlex® microspheres, which are superparamagnetic polystyrene microspheres with surface carboxyl groups and a diameter of about 6.5 μm produced by Luminex Corporation (Austin, Texas). MagPlex® microspheres comprise two or more fluorescent dyes at a specific concentration allowing each microsphere to be identified by a detection system as for instance a MAGPIX® instrument as produced by the Luminex Corporation (Austin, Texas). Microspheres of different MagPlex® microsphere catalog numbers (Luminex Corporation, Austin, Texas) comprise the two or more fluorescent dyes at different concentrations. The MagPlex® microspheres can be excited by the same excitation wavelength and therefore only one light source is required for microsphere identification. In some embodiments, the excitation wavelength is from about 600 to about 650 nm. In some embodiments, the excitation wavelength is from about 615 to about 640 nm. In some embodiments, the excitation wavelength is from about 620 to about 635 nm. For instance, in some embodiments, the excitation wavelength is about 635 nm.

### Norovirus VLP

The norovirus VLP the microsphere is coupled to may be derived from any norovirus of any genogroup, genotype, or variant. Non-limiting examples of suitable norovirus strains include Norwalk virus (NV, GenBank M87661), Southampton virus (SHV, GenBank L07418), Desert Shield virus (DSV, U04469), Hesse virus (HSV), Chiba virus (CHV, GenBank AB042808), Hawaii virus (HV, GenBank U07611), Snow Mountain virus (SMV, GenBank U70059), Toronto virus (TV, Leite et al., Arch. Virol. 141:865-875), Bristol virus (BV), Jena virus (JV, AJ01099), Maryland virus (MV, AY032605), Seto virus (SV, GenBankAB031013), Camberwell (CV, AF145896), Lordsdale virus (LV, GenBank X86557), Grimsby virus (GrV, AJ004864), Mexico virus (MXV, GenBank U22498), Boxer (AF538679), C59 (AF435807), VA115 (AY038598), BUDS (AY660568), Houston virus (HoV), Minerva strain (EF 1269631), Laurens strain (EF 1269661), MOH (AF397156), Parris Island (PiV, AY652979), VA387 (AY038600), VA207 (AY038599), and Operation Iraqi Freedom (OIF, AY675554). Further examples include Hu/GI.1/Norwalk/1968/US (GenBank M87661), Hu/GI.2/Jingzhou/2013401/CHN (GenBank KF306212), Hu/GI.3/JKPG_883/SWE/2007 (GenBank FJ711164.1), Hu/GI.4/1643/2008/US (GenBank GQ413970), Hu/GI.5/Siklos-HUN5407/2013/HUN (Gen Bank KJ402295), Hu/GI.6/TCH-099/USA/2003 (GenBank KC998959), Hu/GI.7/Providence191/2010/USA (GenBank JN899243), Hu/GII.3/NIHIC8.1/2011/USA (GenBank KC597140), Hu/GII.4/Houston/TCH186/2002/US (GenBank JX459908), Hu/GII.4/DenHaag89/2006/NL (GenBank EF126965.1), Hu/GII.4/Yerseke38/2006/NL (GenBank EF126963.1), Hu/GII.4/Sydney/NSW0514/2012/AU (GenBank JX459908), Hu/GII.4/031693/USA/2003 (GenBank JQ965810.1), Hu/GII.6/Ehime120246/2012/JP (GenBank AB818400), Hu/GII.12 strain E5152 (GenBank, Hu/GII.17/C142/GF/1978 (GenBank JN699043), Hu/GII.17/JP/2014/Nagano7-1 (GenBank LC043139), and Hu/GII.17/HKG/2015/CUHK-NS-513 (GenBank KP698931.1). Further examples of noroviruses are Norovirus genogroup 1 strain Hu/NoV/West Chester/2001/USA, GenBank Accession No. AY502016; Norovirus genogroup 2 strain Hu/NoV/Braddock Heights/1999/US A, GenBank Accession No. AY502015; Norovirus genogroup 2 strain Hu/NoV/Fayette/1999/US A, GenBank Accession No. AY502014; Norovirus genogroup 2 strain Hu/NoV/Fairfield/1999/USA, GenBank Accession No. AY502013; Norovirus genogroup 2 strain Hu/NoV/Sandusky/1999/USA, GenBank Accession No. AY502012; Norovirus genogroup 2 strain Hu/NoV/Canton/1999/USA, GenBank Accession No. AY502011; Norovirus genogroup 2 strain Hu/NoV/Tiffin/1999/USA, GenBank Accession No. AY502010; Norovirus genogroup 2 strain Hu/NoV/CS-El/2002/USA, GenBank Accession No. AY50200; Norovirus genogroup 1 strain Hu/NoV/Wisconsin/2001/USA, GenBank Accession No. AY502008; Norovirus genogroup 1 strain Hu/NoV/CS-841/2001/USA, GenBank Accession No. AY502007; Norovirus genogroup 2 strain Hu/NoV/Hiram/2000/USA, GenBank Accession No. AY502006; Norovirus genogroup 2 strain Hu/NoV/Tontogany/1999/USA, GenBank Accession No. AY502005; Norwalk virus, complete genome, GenBank Accession No. NC. sub.—001959; Norovirus Hu/GI/Otofuke/1979/JP genomic RNA, complete genome, GenBank Accession No. AB 187514; Norovirus Hu/Hokkaido/133/2003/JP, GenBank Accession No. AB212306; Norovirus Sydney 2212, GenBank Accession No. AY588132; Norwalk virus strain SN2000JA, GenBank Accession No. AB190457; Lordsdale virus complete genome, GenBank Accession No. X86557; Norwalk-like virus genomic RNA, Gifu'96, GenBank Accession No. AB045603; Norwalk virus strain Vietnam 026, complete genome, GenBank Accession No. AF504671; Norovirus Hu/GI.4/2004/N/L, GenBank Accession No. AY883096; Norovirus Hu/GII/Hokushin/03/JP, GenBank Accession No. AB195227; Norovirus Hu/GII/Kamo/03/JP, GenBank Accession No. AB 195228; Norovirus Hu/GII/Sinsiro/97/JP, GenBank Accession No. AB 195226; Norovirus Hu/GII/Ina/02/JP, GenBank Accession No. AB195225; Norovirus Hu/NLV/GII/Neustrelitz260/2000/DE, GenBank Accession No. AY772730; Norovirus Hu/NLV/Dresdenl 74/pUS-NorII/l 997/GE, GenBank Accession No. AY741811; Norovirus Hu/NLV/Oxford/B2S16/2002/UK, GenBank Accession No. AY587989; Norovirus Hu/NLV/Oxford/B4S7/2002/UK, GenBank Accession No. AY587987; Norovirus Hu/NLV/Witney/B7S2/2003/UK, GenBank Accession No. AY588030; Norovirus Hu/NLV/Banbury/B9S23/2003/UK, GenBank Accession No. AY588029; Norovirus Hu/NLV/ChippingNorton/2003/UK, GenBank Accession No. AY588028; Norovirus Hu/NLV/Didcot/B9S2/2003/UK, GenBank Accession No. AY588027; Norovirus Hu/NLV/Oxford/B8S5/2002/UK, GenBank Accession No. AY588026; Norovirus Hu/NLV/Oxford/B6S4/2003/UK, GenBank Accession No. AY588025; Norovirus Hu/NLV/Oxford/B6S5/2003/UK, GenBank Accession No. AY588024; Norovirus Hu/NLV/Oxford/B5S23/2003/UK, GenBank Accession No. AY588023; Norovirus Hu/NLV/Oxford/B6S2/2003/UK, GenBank Accession No. AY588022; Norovirus Hu/NLV/Oxford/B6S6/2003/UK, GenBank Accession No. AY588021; Norwalk-like virus isolate Bo/ThirsklO/00/UK, GenBank Accession No. AY126468; Norwalk-like virus isolate Bo/Penrith55/00/UK, GenBank Accession No. AY126476; Norwalk-like virus isolate Bo/Aberystwyth24/00/UK, GenBank Accession No. AY 126475; Norwalk-like virus isolate Bo/Dumfries/94/UK, GenBank Accession No. AY126474; Norovirus NLV/IF2036/2003/Iraq, GenBank Accession No. AY675555; Norovirus NLV/IF1998/2003/Iraq, GenBank Accession No. AY675554; Norovirus NLV/BUDS/2002/USA, GenBank Accession No. AY660568; Norovirus NLV/Paris Island/2003/USA, GenBank Accession No. AY652979; Snow Mountain virus, complete genome, GenBank Accession No. AY134748; Norwalk-like virus NLV/Fort Lauderdale/560/1998/US, GenBank Accession No. AF414426; Hu/Norovirus/hiroshima/1999/JP (9912-02F), GenBank Accession No. AB044366; Norwalk-like virus strain 1 IMSU-MW, GenBank Accession No. AY274820; Norwalk-like virus strain B-ISVD, GenBank Accession No. AY274819; Norovirus genogroup 2 strain Hu/NoV/Farmington Hills/2002/USA, GenBank Accession No. AY502023; Norovirus genogroup 2 strain Hu/NoV/CS-G4/2002/USA, GenBank Accession No. AY502022; Norovirus genogroup 2 strain Hu/NoV/CS-G2/2002/USA, GenBank Accession No. AY502021; Norovirus genogroup 2 strain Hu/NoV/CS-G12002/USA, GenBank Accession No. AY502020; Norovirus genogroup 2 strain Hu/NoV/Anchorage/2002/USA, GenBank Accession No. AY502019; Norovirus genogroup 2 strain Hu/NoV/CS-Dl/2002/CAN, GenBank Accession No. AY502018; Norovirus genogroup 2 strain Hu/NoV/Germanton/2002/USA, GenBank Accession No. AY502017; Human calicivirus NLV/GII/Langen1061/2002/DE, complete genome, GenBank Accession No. AY485642; Murine norovirus 1 polyprotein, GenBank Accession No. AY228235; Norwalk virus, GenBank Accession No. AB067536; Human calicivirus NLV/Mex7076/1999, GenBank Accession No. AF542090; Human calicivirus NLV/Oberhausen 455/01/DE, GenBank Accession No. AF539440; Human calicivirus NLV/Herzberg 385/01/DE, GenBank Accession No. AF539439; Human calicivirus NLV/Boxer/2001/US, GenBank Accession No. AF538679; Norwalk-like virus genomic RNA, complete genome, GenBank Accession No. AB081723; Norwalk-like virus genomic RNA, complete genome, isolate: Saitama U201, GenBank Accession No. AB039782; Norwalk-like virus genomic RNA, complete genome, isolate: Saitama Ul 8, GenBank Accession No. AB039781; Norwalk-like virus genomic RNA, complete genome, isolate: Saitama U25, GenBank Accession No. AB039780; Norwalk virus strain:U25GII, GenBank Accession No. AB067543; Norwalk virus strain:U201 GII, GenBank Accession No. AB067542; Norwalk-like viruses strain 416/97003156/1996/LA, GenBank Accession No. AF080559; Norwalk-like viruses strain 408/97003012/1996/FL, GenBank Accession No. AF080558; Norwalk-like virus NLV/Burwash Landing/331/1995/US, GenBank Accession No. AF414425; Norwalk-like virus NLV/Miami Beach/326/1995/US, GenBank Accession No. AF414424; Norwalk-like virus NLV/White River/290/1994/US, GenBank Accession No. AF414423; Norwalk-like virus NLV/New Orleans/306/1994/US, GenBank Accession No. AF414422; Norwalk-like virus NLV/Port Canaveral/301/1994/US, GenBank Accession No. AF414421; Norwalk-like virus NLV/Honolulu/314/1994/US, GenBank Accession No. AF414420; Norwalk-like virus NLV/Richmond/283/1994/US, GenBank Accession No. AF414419; Norwalk-like virus NLV/Westover/302/1994/US, GenBank Accession No. AF414418; Norwalk-like virus NLV/UK3—17/12700/1992/GB, GenBank Accession No. AF414417; Norwalk-like virus NLV/Miami/81/1986/US, GenBank Accession No. AF414416; Snow Mountain strain, GenBank Accession No. U70059; Desert Shield virus DSV395, GenBank Accession No. U04469; Norwalk virus, complete genome, GenBank Accession No. AF093797; Hawaii calicivirus, GenBank Accession No. U07611; Southampton virus, GenBank Accession No. L07418; Norwalk virus (SRSV-KY-89/89/J), GenBank Accession No. L23828; Norwalk virus (SRSV-SMA/76/US), GenBank Accession No. L23831; Camberwell virus, GenBank Accession No. U46500; Human calicivirus strain Melksham, GenBank Accession No. X81879; Human calicivirus strain MX, GenBank Accession No. U22498; Minireovirus TV24, GenBank Accession No. U02030; and Norwalk-like virus NLV/Gwynedd/273/1994/US, GenBank Accession No. AF414409; sequences of all of which (as entered by the date of filing of this application) are herein incorporated by reference. Additional Norovirus sequences are disclosed in the following patent publications: WO 2005/030806, WO 2000/79280, JP2002020399, US2003129588, U.S. Pat. No. 6,572,862, WO 1994/05700, and WO 05/032457, all of which are herein incorporated by reference in their entireties. See also Green et al. (2000) J. Infect. Dis., Vol. 181 (Suppl. 2):S322-330; Wang et al. (1994) J. Virol, Vol. 68:5982-5990; Chen et al. (2004) J. Virol, Vol. 78: 6469-6479; Chakravarty et al. (2005) J. Virol, Vol. 79: 554-568; Hansman et al. (2006) J. Gen. Virol, Vol. 87:909-919; Bull et al. (2006) J. Clin. Micro., Vol. 44(2):327-333; Siebenga, et al. (2007) J. Virol, Vol. 81(18):9932-9941, and Fankhauser et al. (1998) J. Infect. Dis., Vol. 178: 1571-1578; for sequence comparisons and a discussion of genetic diversity and phylogenetic analysis of noroviruses. The present disclosure is also directed to noroviruses that have not been characterized or discovered at the time of filing or may emerge after the time of filing.

The norovirus VLP may further be derived from a norovirus consensus sequence from two or more noroviruses such as GII.4 variants. The norovirus VLP derived from such a norovirus consensus sequence has antigenic properties of the two or more noroviruses. Consensus sequences may be determined from any noroviruses. The consensus sequence may be derived from sequences encoding structural proteins of the noroviruses, in particular sequences encoding VP1 proteins of the noroviruses (see also Example 1 below). In one embodiment, the consensus sequence is constructed from the VP1 sequences of GII.4 noroviruses: Hu/GII.4/Houston/TCH186/2002/US, Hu/GII.4/DenHaag89/2006/NL, and Hu/GII.4/Yerseke38/2006/NL as described for instance in WO 2010/0175242 and Parra et al., Vaccine 2012, 30(24):350-3586. The resulting norovirus VLP is designated in the application as GII.4/Consensus VLP (cf. also Table 1). In another embodiment the consensus sequence is constructed from the sequences of GI noroviruses: Norwalk virus (Accession Number: M87661), Southampton (Accession Number: Q04542), and Chiba virus (Accession Number: BAB18267).

The norovirus VLP comprises at least one of the structural proteins (VP1, VP2) of the norovirus from which it is derived. In one embodiment the norovirus VLP contains the major structural protein (VP1) and the minor structural protein (VP2). In more specific embodiments the norovirus VLP contains the major structural protein (VP1). According to one specific embodiment, the norovirus VLP comprises the major structural protein (VP1) which is at least 80%, or at least 85%, or at least 90%, or at least 95% or 100% identical to the VP1 sequence from the norovirus from which the norovirus VLP is derived.

The norovirus VLP of the present disclosure can either comprise one or more full length structural proteins of the norovirus from which it is derived or truncated versions thereof. A truncated version may be a certain domain of the structural protein.

According to one embodiment the norovirus VLP of the present disclosure comprises one or more structural proteins of one norovirus. According to another embodiment the norovirus VLP of the present disclosure comprises one or more structural proteins of at least two different noroviruses. For instance, a norovirus VLP may comprise the VP1 protein from one norovirus strain and the VP1 protein from another norovirus strain.

The norovirus VLP is produced recombinant in an expression system using a norovirus nucleic acid sequence, which encodes at least one capsid protein or truncated version thereof. Once coding sequences for the desired particle-forming polypeptides have been isolated or synthesized, they can be cloned into any suitable vector or replicon for expression. Numerous cloning vectors are known to those of skill in the art, and the selection of an appropriate cloning vector is within the skill of an ordinary artisan. The vector is then used to transform an appropriate host cell. Suitable recombinant expression systems include, but are not limited to, bacterial (e.g. E. coli, Bacillus subtilis, and Streptococcus), baculovirus/insect, vaccinia, Semliki Forest virus (SFV), Alphaviruses (such as, Sindbis, Venezuelan Equine Encephalitis (VEE)), mammalian (e.g. Chinese hamster ovary (CHO) cells, Vero cells, HEK-293 cells, HeLa cells, baby hamster kidney (BHK) cells, mouse myeloma (SB20), and monkey kidney cells (COS)), yeast (e.g. S. cerevisiae, S. pombe, Pichia pastori and other Pichia expression systems), plant, and Xenopus expression systems, as well as others known in the art.

In some embodiments, the Norovirus VLPs are used in the substantially pure state. Depending on the expression system and host selected, VLPs are produced by growing host cells transformed by an expression vector under conditions whereby the particle-forming polypeptide is expressed and VLPs can be formed. The selection of the appropriate growth conditions is within the skill of the art.

According to one embodiment of the disclosure the norovirus VLP is produced recombinant in a eukaryotic expression system.

In a particular embodiment the norovirus VLP is produced in a mammalian expression system. The procedures for producing VLPs in mammalian cell culture are well known in the art. For instance, recombinant adenovirus clones carrying the mammalian codon optimized nucleotide sequences encoding for structural proteins are used to infect mammalian cells such as Vero cells. VLPs can be isolated from cell culture.

In other particular embodiments the norovirus VLP is produced in an insect expression system. Suitable insect cells include Sf9, High Five, TniPro, Aedes aegypti, Autographa californica, Bombyx mori, Drosophila melanogaster, Spodoptera frugiperda, and Trichoplusia ni. The procedures for producing VLPs in insect cell culture are well known in the art (see, for example, U.S. Pat. No. 6,942,865, which is incorporated herein by reference in its entirety). Briefly, the recombinant baculoviruses carrying the capsid sequence are constructed from the synthetic cDNAs. The recombinant baculovirus are then used to infect insect cell cultures (e.g. Sf9, High Five and TniPro cells) and VLPs can be isolated from the cell culture.

If the VLPs are formed intracellularly, the cells are then disrupted, using chemical, physical or mechanical means, which lyse the cells yet keep the VLPs substantially intact. Such methods are known to those of skill in the art and are described in, e.g., Protein Purification Applications: A Practical Approach, (E. L. V. Harris and S. Angal, Eds., 1990).

The particles are then isolated (or substantially purified) using methods that preserve the integrity thereof, such as, by density gradient centrifugation, e.g., sucrose gradients, PEG-precipitation, pelleting, and the like (see, e.g., Kirnbauer et al. J. Virol. (1993) 67:6929-6936), as well as standard purification techniques including, e.g., ion exchange and gel filtration chromatography.

General texts which describe molecular biological techniques, which are applicable to the present disclosure, such as cloning, mutation, and the like, include Berger and Kimmel, Guide to Molecular Cloning Techniques, Methods in Enzymology volume 152 Academic Press, Inc., San Diego, Calif. (Berger); Sambrook et al, Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al, eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and expression of capsid proteins of noroviruses.

Throughout the application and the claims, specific norovirus VLPs are referred to as designated in Table 1. For instance, a GI.1 VLP refers to a norovirus VLP derived from the Hu/GI.1/Norwalk/1968/US norovirus, i.e. comprising the major capsid protein VP1 with a sequence that is at least 80%, or at least 85%, or at least 90%, or at least 95% or 100% identical to the sequence provided in GenBank: AAB50466.2 and SEQ ID NO: 1.

### Reporter Antibody, Secondary Reporter Antibody, and Detection Antibody

Specifications and embodiments within this section can be combined with any of the specifications and embodiments of the previous and following sections.

The reporter, secondary reporter, and detection Abs for use in the methods and kits of the present disclosure may be recombinant Abs, monoclonal Abs, or polyclonal Abs.

According to certain embodiments of the disclosure the reporter, the secondary reporter, and the detection antibody are full-length immunoglobulin (Ig) molecules, comprising four polypeptide chains, two heavy (H) chains and two light (L) chains interconnected by disulfide bonds.

The reporter, secondary reporter, and detection Ab may be derived from any origin. According to certain embodiments of the disclosure the reporter, the secondary reporter, and the detection Ab are derived from a non-human origin such as sheep, mouse, rabbit, goat, or donkey. In certain embodiments the reporter Ab may be derived from a human origin. “Derived from” within this context means that the Ab was produced in the corresponding origin. For instance, an Ab derived from sheep, refers to an Ab, which was produced in sheep.

### Detection Ab

The detection Ab is applied in methods of the present disclosure, which make use of a non-competitive microsphere immunoassay set-up, wherein one or more norovirus VLPs to which one or more different microspheres are coupled, are contacted with the sample (reference is also made to the sub-section “Non-competitive microsphere immunoassay set-up” below).

According to the disclosure, the detection Ab is capable of binding to norovirus-reactive antibodies in a sample. In particular embodiments, the detection Ab is capable of binding to the heavy chain constant region of norovirus-reactive antibodies in a sample with the variable region of the detection Ab.

In certain embodiments the detection Ab binds to antibodies from the isotype A (IgA) and does not bind to antibodies from other isotypes. In one embodiment the reporter Ab binds to antibodies from the isotype G (IgG) and does not bind to antibodies from other isotypes. In one embodiment the reporter Ab binds to antibodies from the isotype M (IgM) and does not bind to antibodies from other isotypes. In one embodiment the reporter Ab binds to antibodies from the isotype A, G, and M (IgA, IgG, and IgM).

Within the embodiments of the present disclosure, the detection Ab is (directly) attached to a detectable label. In some embodiments, the detection Ab is attached to the detectable label by the heavy chain constant region of the detection Ab.

Concerning the detectable label, the detection Ab is attached to, reference is also made to the sub-section “Detectable label”.

### Reporter Ab

The reporter Ab is applied in methods of the present disclosure, which make use of a competitive microsphere immunoassay set-up, wherein one or more norovirus VLPs to which one or more different microspheres are coupled, are contacted with the sample (reference is also made to the sub-section “Competitive microsphere immunoassay set-up” below).

Within these embodiments, the reporter Ab is capable of binding to the one or more norovirus VLPs. In particular embodiments the reporter Ab binds to the one or more norovirus VLPs with the variable region of the reporter antibody. Thereby, the reporter Ab is capable of competing with the norovirus-reactive Abs in the sample for binding to the one or more norovirus VLPs.

In one embodiment the reporter Ab is a monoclonal antibody. In one embodiment the reporter Ab is derived from a non-human origin. In one embodiment the reporter Ab is a norovirus-neutralizing antibody. In one embodiment the reporter Ab is a norovirus-blocking antibody. In one embodiment the reporter Ab is a norovirus-blocking antibody and a norovirus-neutralizing antibody.

In some embodiments, wherein two or more norovirus VLPs are contacted with the sample (multiplexing methods), the reporter antibody only binds to one of the two or more norovirus VLPs and does not bind to the other norovirus VLPs. In certain other embodiments, wherein two or more norovirus VLPs are contacted with the sample (multiplexing methods), the reporter antibody binds to more than one of the two or more norovirus VLPs, i.e. the reporter Ab is a cross-reactive reporter Ab. For instance, mAb 5A04 solely binds to GII.4/Sydney VLP and provides an EC₅₀ value towards the GII.4/Sydney VLP of 0.004 μg/mL. In contrast, mAb 8A08 provides an EC₅₀ value towards GII.4/Den Haag and GII.4/New Orleans of 0.009 or 0.008 μg/mL, respectively, and is therefore a cross-reactive reporter Ab (cf. Table 10).

In some embodiments, the reporter Ab comprises a heavy chain variable region (VH) amino acid sequence as represented by SEQ ID NO: 27, and a light chain variable region (VL) amino acid sequence as represented by SEQ ID NO: 28. For corresponding DNA sequences and full-length sequences, reference is also made to the Annex section in the Examples below.

In some embodiments, the reporter Ab comprises a heavy chain variable region (VH) amino acid sequence as represented by SEQ ID NO: 29, and a light chain variable region (VL) amino acid sequence as represented by SEQ ID NO: 30. For corresponding DNA sequences and full-length sequences, reference is also made to the Annex section in the Examples below.

By application of a cross-reactive reporter antibody reactive to different norovirus VLPs contacted with the sample, antibodies within the sample that are also cross-reactive to the different norovirus VLPs can be determined by application of one reporter Ab.

In one embodiment the reporter Ab provides an EC₅₀ value towards the one or more norovirus VLPs applied in the methods of the present disclosure of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.03 μg/mL or less than 0.02 μg/mL or less than 0.01 μg/mL.

In some embodiments, the reporter Ab is attached to a detectable label. In some embodiments, the reporter antibody is attached to a detectable label by the heavy chain constant region of the reporter Ab.

In certain embodiments the reporter Ab is directly (i.e. itself) attached to a detectable label. In some embodiments, the reporter Ab is directly attached to the detectable label by the heavy chain constant region of the reporter Ab. In embodiments, wherein the reporter Ab is itself attached to a detectable label, no secondary reporter Ab is necessary.

In certain embodiments the reporter Ab is not directly, but indirectly attached to a detectable label. In these embodiments, a secondary reporter Ab is applied in order to enable detection of the reporter Ab. In some embodiments, the reporter Ab is indirectly attached to the detectable label by the heavy chain constant region of the at least one reporter antibody wherein the reporter antibody reacts with a secondary reporter antibody directly attached to a detectable label.

Concerning the secondary reporter antibody, reference is made to the sub-section “Secondary reporter Ab” below. Concerning the detectable label, the reporter Ab is attached to, reference is also made to the sub-section “Detectable label”.

### Secondary Reporter Ab

In embodiments, wherein the reporter antibody is indirectly attached to a detectable label, the reporter antibody is detected by incubation with a secondary reporter Ab, wherein the secondary reporter Ab binds to the reporter Ab. In some embodiments, the secondary reporter Ab binds to the heavy chain constant region of the reporter Ab with the variable region of the secondary reporter Ab.

Within the meaning of the disclosure, the secondary reporter Ab is (directly) attached to a detectable label, e.g., via the heavy chain constant region of the secondary reporter Ab.

Concerning the detectable label, the reporter Ab is attached to, reference is also made to the sub-section “Detectable label”.

### Detectable Label

According to one embodiment of the disclosure the detectable label to which the detection, the reporter, and the secondary reporter antibody are attached to is a compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik). The detectable label according to the present disclosure can be detected with a suitable detection system.

According to one embodiment of the disclosure the detectable label is selected from the group consisting of fluorescent labels, magnetic labels, enzyme labels, colored labels, chromogenic labels, luminescent labels, radioactive labels, haptens, biotin, metal complexes, metals, and colloidal gold. All these types of labels are well established in the art.

According to one embodiment of the disclosure the detectable label is selected from such which provide the emission of fluorescence or phosphorescence upon irradiation or excitation or the emission of X-rays when using a radioactive label.

According to one embodiment of the disclosure the detectable label is an enzyme label, which include but are not limited to alkaline phosphatase, horseradish peroxidase (HRP), β-galactosidase, and β-lactamase. Enzyme labels catalyze the formation of chromogenic reaction products.

In specific embodiments the detectable labels are fluorescent labels. Numerous fluorescent labels are well established in the art and commercially available from different suppliers (see, for example, The Handbook—A Guide to Fluorescent Probes and Labeling Technologies, 10th ed. (2006), Molecular Probes, Invitrogen Corporation, Carlsbad, CA, USA). Examples of fluorescent labels include but are not limited to xanthene, fluorescein isothiocyanate, rhodamine, phycoerythrin (PE), cyanine, coumarin, and any derivative thereof. According to some embodiments of the disclosure, the fluorescent label is PE.

In the embodiments wherein the detectable label to which the detection and/or reporter Ab is attached to is a fluorescent label, the fluorescent label can be irradiated/excited with any suitable light source present within a detection system. The light source may be a laser or a LED. In the case the fluorescent label is PE, the excitation wavelength of the light source is in the range of about 505 to about 535 nm, for instance about 511 nm.

Concerning the detection of such detectable labels with suitable detection systems, reference is also made to the section “Detection system”.

### Detection System

According to the present disclosure, the detection system refers to any system which is suitable for determining values indicative for the presence and/or amount of a reporter antibody or a secondary reporter antibody or a detection antibody.

According to the present disclosure, the detection system may also be able to determine values indicative for the presence and/or amount of a microsphere.

The selection of a suitable detection system depends on several parameters such as the type of detectable labels used for detection, or the kind of analysis performed. Various optical and non-optical detection systems are well established in the art. A general description of detection systems that can be used with the method can be found, e.g., in Lottspeich, F., and Zorbas H., Springer Spektrum 2012, Bioanalytik.

According to one embodiment of the disclosure, the detection system is an optical detection system. In some embodiments, performing the method involves detection systems, which may be based on the measurement of parameters such as fluorescence, optical absorption, resonance transfer, and the like.

According to one embodiment of the disclosure, the detection system measures fluorescence. Such systems measure the capacity of particular molecules to emit their own light when excited by light of a particular wavelength resulting in a characteristic absorption and emission behavior. In particular, quantitative detection of fluorescence signals is performed by means of modified methods of fluorescence microscopy (for review see, e.g., Lichtman, J. W., and Conchello, J. A. (2005) Nature Methods 2, 910-919; Zimmermann, T. (2005) Adv. Biochem. Eng. Biotechnol. 95, 245-265). Thereby, the signals resulting from light absorption and light emission, respectively, are separated by one or more filters and/or dichroites and imaged on suitable detectors. Data analysis is performed by means of digital image processing. Image processing may be achieved with several software packages well known in the art (such as Mathematica Digital Image Processing, EIKONA, or Image-PRO). Another suitable software for such purposes is the Iconoclust software (Clondiag Chip Technologies GmbH, Jena, Germany). Suitable detection systems may be based on “classical” methods for measuring a fluorescent signal such as epifluorescence or darkfield fluorescence microscopy (reviewed, e.g., in: Lakowicz, J. R. (1999) Principles of Fluorescence Spectroscopy, 2nd ed., Plenum Publishing Corp., NY). Another optical detection system that may be used is confocal fluorescence microscopy, wherein the object is illuminated in the focal plane of the lens by a point light source. Importantly, the point light source, object and point light detector are located on optically conjugated planes. Examples of such confocal systems are described in detail, for example, in Diaspro, A. (2002) Confocal and 2-photon-microscopy: Foundations, Applications and Advances, Wiley-Liss, Hobroken, NJ. The fluorescence-optical system is usually a fluorescence microscope without an autofocus, for example a fluorescence microscope having a fixed focus. Further fluorescence detection methods that may also be used include inter alia total internal fluorescence microscopy (see, e.g., Axelrod, D. (1999) Surface fluorescence microscopy with evanescent illumination, in: Lacey, A. (ed.) Light Microscopy in Biology, Oxford University Press, New York, 399-423), fluorescence lifetime imaging microscopy (see, for example, Dowling, K. et al. (1999) J. Mod. Optics 46, 199-209), fluorescence resonance energy transfer (FRET; see, for example, Periasamy, A. (2001) J. Biomed. Optics 6, 287-291), bioluminescence resonance energy transfer (BRET; see, e.g., Wilson, T., and Hastings, J. W. (1998) Annu. Rev. Cell Dev. Biol. 14, 197-230), and fluorescence correlation spectroscopy (see, e.g., Hess, S. T. et al. (2002) Biochemistry 41, 697-705). In some embodiments, detection is performed using FRET or BRET, which are based on the respective formation of fluorescence or bioluminescence quencher pairs. The use of FRET is also described, e.g., in Liu, B. et al. (2005) Proc. Natl. Acad. Sci. USA 102, 589-593; and Szollosi, J. et al. (2002) J. Biotechnol. 82, 251-266. The use of BRET is detailed, for example, in Prinz, A. et al. (2006) Chembiochem. 7, 1007-1012; and Xu, Y. et al. (1999) Proc. Natl. Acad. Sci. USA 96, 151-156.

In one embodiment the detection system comprises a first light source, e.g. an argon laser or a light emitting diode (LED), which has an excitation wavelength in the range of about 400 to about 700 nm and a second light source, e.g. an argon laser or a LED, which has an excitation wavelength in the range of about 300 to about 700 nm and a suitable detection component as for instance a photodiode such as an avalanche photodiode (APD) in combination with a photomultiplier or a charge-coupled device (CCD) sensor. The first light source may be used for the identification of the detectable label of a microsphere, wherein the detectable label may be one or more fluorescent dyes at a specific concentration. The second light source may be used for excitation of the detectable label of a reporter or a secondary reporter or a detection antibody.

In some embodiments, the first light source, e.g. the argon laser or LED, has an excitation wavelength in the range of about 600 to about 650 nm and the second light source, e.g. the argon laser or LED, has an excitation wavelength in the range of about 500 to about 600 nm. In some embodiments, the first light source, e.g. the argon laser or LED, has an excitation wavelength in the range of about 615 to about 640 nm and the second light source, e.g. the argon laser or LED, has an excitation wavelength in the range of about 505 to about 540 nm. In some embodiments, the first light source, e.g. the argon laser or LED, has an excitation wavelength in the range of about 620 to about 635 nm and the second light source, e.g. the argon laser or LED, has an excitation wavelength in the range of about 510 to about 535 nm. For instance, the detection system comprises a first light source, e.g. an argon laser or a LED, which has an excitation wavelength of about 635 nm and a second light source, e.g. an argon laser or a LED, which has an excitation wavelength of about 525 nm.

The detection system may be also capable of distinguishing the individual size or shape of a microsphere from the individual size or shape of another microsphere, thereby allowing individual identification of the microsphere.

The detection system may be one of the group consisting of MAGPIX®, Luminex 200®, and FLEXMAP 3D® (Luminex Corp. Austin, Tex.). In some embodiments, the detection system is the MAGPIX® (Luminex Corp. Austin, Tex.).

The detection system may be operated by a specific software, including the xPONENT® software (Luminex Corp. Austin, Tex.).

The detection system may be capable of detecting both, the signal from the detectable label of the reporter or secondary reporter or detection Ab, as well as the signal from the detectable label of the microsphere.

The detection system may be capable of analyzing one microsphere after the other thereby identifying the microsphere by detecting the signal from the detectable label of the microsphere and detecting the signal from the detectable label of the reporter or secondary reporter or detection antibody such as flow cytometry-based detection systems (e.g. Luminex 200® and FLEXMAP 3D®). The flow cytometry-based detection systems Luminex 200® and FLEXMAP 3D® include two lasers each one for irradiation of the detectable label of the microsphere and the detectable label of the reporter or secondary reporter or detection Ab. As flow cytometry-based detection systems are not capturing the microspheres with a magnet, the Luminex 200® and FLEXMAP 3D® systems are compatible with both, magnetic microspheres such as the MagPlex® microspheres and non-magnetic microspheres such as the Microplex® microspheres. The Luminex 200® and FLEXMAP 3D® systems detect signals from the microspheres and reporter or secondary reporter or detection Ab by avalanche photodiodes (APD) in combination with photomultipliers (PMT).

Alternatively, the detection system may be capable of analyzing multiple microspheres at once. Therefore, a monolayer of magnetic microspheres is captured by a magnet and the microspheres are excited with two LEDs, one LED for excitement of the detectable labels of the microspheres and the other LED for excitement of the detectable label of the reporter or detection Ab. The signals from the microspheres and reporter or detection Ab are recorded by a CCD imager, which allows identification of each microsphere and the corresponding antigen to which the microsphere is coupled to. An example for a LED-based detection system is the MAGPIX® instrument. As analyses with the MAGPIX® instrument involves capture of the microspheres with a magnet, the MAGPIX® instrument is solely compatible with magnetic microspheres such as MagPlex® microspheres.

### Sample and Subject

The following section is intended to specify the sample and subject referred to in the methods of the present disclosure. Specifications and embodiments within this section can be combined with any of the specifications and embodiments of the previous and following sections.

### Sample

According to the present disclosure the sample may be any sample derived from a subject. In some embodiments, the sample is selected from the group consisting of blood, urine, saliva, cerebrospinal fluid, and lymph fluid. In particular embodiments the sample is a serum or blood plasma sample.

In certain embodiments the sample comprises norovirus-reactive Abs capable of binding to the norovirus VLPs applied in the methods of the present application.

In certain embodiments the sample may be pre-treated prior to use in the methods of the present disclosure. Methods for pre-treating can involve purification, filtration, distillation, concentration, inactivation of interfering compounds, and the addition of reagents.

In particular embodiments the sample is heat-inactivated e.g. for about 30 to 90 minutes at about 55 to about 65° C. In general, heat-inactivation can be varied according to the type of sample to be analyzed.

In more particular embodiments the sample is a heat-inactivated serum or blood plasma sample.

### Subject

In one embodiment of the disclosure, the subject is a mammal. In specific embodiments the mammal is selected from the group consisting of a mouse, a primate, a non-human primate, a human, a rabbit, a cat, a rat, a horse, a sheep.

In certain embodiments the subject is a pregnant mammal, and in particular embodiments a pregnant woman.

In other embodiments the subject is a newborn up to 2 months of age or a child, the child being 2 months to 5 years of age. The subject might also be 70 years or older.

In some embodiments the subject is a patient, for whom prophylaxis or therapy is desired.

In some embodiments the subject is norovirus naïve, or norovirus exposed.

In some embodiments the subject is from a norovirus endemic region or a norovirus non-endemic region. In some embodiments the subject is from a norovirus non-endemic region travelling to a norovirus endemic region.

In some embodiments, the subject is vaccinated with a norovirus vaccine.

### Methods for Determining Norovirus-Reactive Antibodies

The present disclosure is further directed to various methods for determining norovirus-reactive antibodies using non-competitive and competitive microsphere immunoassay set-ups. Regarding the microsphere complex, the detection system, the reporter, secondary reporter and detection antibody, as well as the subject and sample, reference is made to the respective chapters above. In addition, certain specific embodiments are also outlined in this section and shall, however, not be taken as limiting. Any embodiments from this section can be combined with any of the embodiments from the previous or following sections.

### Non-Competitive Microsphere Immunoassay Set-Ups

In a non-competitive microsphere immunoassay set-up, no reporter antibody is applied. In order to detect the norovirus-reactive antibodies in the sample a detection Ab is used. The non-competitive microsphere immunoassay set-up can be modified to enable determination of norovirus-reactive antibodies against one norovirus VLP (singleplex assay set-up) or to enable concomitant determination of antibodies reactive to two or more norovirus VLPs in one single experiment (multiplex assay set-up).

Method for Determining the Presence and/or Amount of Norovirus-Reactive Antibodies (Singleplex Assay Set-Up)

The method for determining the presence and/or amount of norovirus-reactive antibodies in a sample from a subject comprises the steps of:
Step 1: contacting an amount of a microsphere complex as described above with the sample to allow binding of the norovirus-reactive antibodies in the sample to the norovirus virus like particles (VLPs) coupled to the microspheres in the microsphere complex,
Step 2: contacting an amount of a detection antibody with the norovirus-reactive antibodies bound to the norovirus VLPs in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a detectable label, and
Step 3: detecting a signal from the detection antibody bound to the norovirus-reactive antibodies in step 2,
and wherein the method optionally comprises the further steps of:
Step 4: determining the presence and/or amount of the detection antibody bound to the norovirus-reactive antibodies from the signal of step 3, and
Step 5: determining the presence and/or amount of norovirus-reactive antibodies in the sample from the presence and/or amount of the detection antibody determined in step 4.

In some embodiments, the signal from the detection antibody in step 3 is resulting from the detectable label the detection antibody is attached to.

In some embodiments, contacting in step 1 is carried out for about 1 to about 24 hours. In some embodiments, contacting in step 1 is carried out for about 90 minutes. In some embodiments, contacting in step 1 is carried out for about 18 to about 24 hours, e.g., in some embodiments, for about 21 hours.

In some embodiments, contacting in step 1 is carried out at a temperature of about 2 to about 30° C. In some embodiments, contacting in step 1 is carried out at a temperature of about 22° C. In other specific embodiments, contacting in step 1 is carried out at a temperature of about 2 to about 8° C.

In some embodiments, contacting in step 2 is carried out for about 30 to about 90 minutes, e.g., in some embodiments, for about 60 minutes.

Method for Concomitant Determination of the Presence and/or Amount of Antibodies Reactive to Different Noroviruses (Multiplex Assay Set-Up)

The method for concomitant determination of the presence and/or amount of antibodies reactive to different noroviruses in a sample from a subject comprises the steps of:
Step 1: contacting an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label,
with the sample to allow binding of the norovirus-reactive antibodies in the sample to the first and/or the second norovirus VLP,
Step 2: contacting an amount of a detection antibody with the norovirus-reactive antibodies bound to the first and/or the second norovirus VLP in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a third detectable label,
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the detection antibody bound to the norovirus-reactive antibodies bound to the norovirus VLP of the at least one microsphere in step 2 upon irradiation with a second light source,
Step 4: repeating step 3 until at least 30 microspheres coupled to the same norovirus VLP are identified, and
Step 5: summarizing the detected signal from the detection antibody in step 3 for all identified microspheres coupled to the same norovirus VLP, wherein the summarized signal is indicative for the presence and/or amount of norovirus-reactive antibodies in the sample,
and wherein the method optionally comprises the further steps of:
Step 6: determining the presence and/or amount of the detection antibody bound to the norovirus-reactive antibodies from the summarized signal of step 5, and
Step 7: determining the presence and/or amount of norovirus-reactive antibodies in the sample from the presence and/or amount of the detection antibody determined in step 6.

In some embodiments, in step 1 an amount of at least five or at least ten or at least fifteen or at least twenty microsphere complexes is contacted with the sample. Suitable microsphere complex comprise, for instance, microspheres coupled to norovirus VLPs as described in Table 1.

In a particular embodiment, in step 1 an amount of a first microsphere complex comprising a first microsphere coupled to a GI.1 VLP, an amount of a second microsphere complex comprising a second microsphere coupled to a GI.2 VLP, an amount of a third microsphere complex comprising a third microsphere coupled to a GI.3 VLP, an amount of a fourth microsphere complex comprising a fourth microsphere coupled to GI.4 VLP, an amount of a fifth microsphere complex comprising a fifth microsphere coupled to a GI.5 VLP, an amount of a sixth microsphere complex comprising a sixth microsphere coupled to a GI.6 VLP, an amount of a seventh microsphere complex comprising a seventh microsphere coupled to a GI.7 VLP, an amount of an eight microsphere complex comprising an eight microsphere coupled to GII.1 VLP, an amount of a ninth microsphere complex comprising a ninth microsphere coupled to a GII.2 VLP, an amount of a tenth microsphere complex comprising a tenth microsphere coupled to a GII.3 VLP, an amount of an eleventh microsphere complex comprising an eleventh microsphere coupled to a GII.4/Consensus VLP, an amount of a twelfth microsphere complex comprising a twelfth microsphere coupled to GII.4/Sydney VLP, an amount of a thirteenth microsphere complex comprising a thirteenth microsphere coupled to a GII.4/New Orleans VLP, an amount of a fourteenth microsphere complex comprising a fourteenth microsphere coupled to a GII.4/Yerseke VLP, an amount of a fifteenth microsphere complex comprising a fifteenth microsphere coupled to a GII.4/Den Haag VLP, an amount of a sixteenth microsphere complex comprising a sixteenth microsphere coupled to GII.6 VLP, an amount of a seventeenth microsphere complex comprising a seventeenth microsphere coupled to a GII.7 VLP, an amount of an eighteenth microsphere complex comprising an eighteenth microsphere coupled to a GII.12 VLP, an amount of a nineteenth microsphere complex comprising a nineteenth microsphere coupled to a GII.17/1978 VLP, and an amount of a twentieth microsphere complex comprising a twentieth microsphere coupled to GII.17/2015 VLP is contacted with the sample.

In some embodiments, the signal from the detection antibody in step 3 is resulting from the detectable label the detection antibody is attached to.

In some embodiments, contacting in step 1 is carried out for about 1 to about 24 hours. In some embodiments, contacting in step 1 is carried out for about 90 minutes. In other specific embodiments, contacting in step 1 is carried out for about 18 to about 24 hours, e.g., in some embodiments, for about 21 hours.

In some embodiments, contacting in step 1 is carried out at a temperature of about 2 to about 30° C. In some embodiments, contacting in step 1 is carried out at a temperature of about 22° C. In other specific embodiments, contacting in step 1 is carried out at a temperature of about 2 to about 8° C.

In some embodiments, contacting in step 2 is carried out for about 30 to about 90 minutes, e.g., in some embodiments, for about 60 minutes.

In some embodiments, step 4 is repeated until at least 35, at least 40, at least 45, or at least 50 microspheres coupled to the same norovirus VLP are identified.

In some embodiments, the methods for determining norovirus-reactive antibodies in a non-competitive microsphere immunoassay set up as described above (Singleplex and multiplex set-up) can be applied to determine the presence and/or amount of norovirus-reactive antibodies in B cell or hybridoma supernatant samples (see also Example 5 below). In these embodiments, the norovirus-reactive antibodies may be monoclonal antibodies. Thereby, supernatant samples can be screened for the presence of certain norovirus-reactive antibodies. Of particular practical advantage is the screening of hybridoma or B cell supernatants in the multiplex set-up, as the supernatants can be evaluated for antibodies differing in their specificity in one single assay.

### Competitive Microsphere Immunoassay Set-Ups

In a competitive microsphere immunoassay set-up one or more reporter antibodies are applied. The competitive microsphere immunoassay set-up can be modified to enable determination of norovirus-reactive antibodies against one norovirus VLP (singleplex assay set-up) or to enable concomitant determination of antibodies reactive to two or more norovirus VLPs in one single experiment (multiplex assay set-up).

Method for Determining the Presence and/or Amount of Norovirus-Reactive Antibodies (Singleplex Assay Set-Up)

The method for determining the presence and/or amount of norovirus-reactive antibodies in a sample from a subject comprises the steps of:
Step 1: providing a kit, including an amount of a microsphere complex as described above and an amount of a reporter antibody as described above, wherein the reporter antibody binds to the norovirus VLP of the microsphere complex,
Step 2: contacting the amount of the microsphere complex and the amount of the reporter antibody with the sample to allow binding of the norovirus-reactive antibodies in the sample to the norovirus VLPs coupled to the microspheres in the microsphere complex while competing with the reporter antibody, and
Step 3: detecting a signal from the reporter antibody bound to the norovirus VLPs in step 2,
and wherein the method optionally comprises the further steps of:
Step 4: determining the presence and/or amount of the reporter antibody from the signal of step 3, and
Step 5: determining the presence and/or amount of norovirus-reactive antibodies in the sample from the presence and/or amount of the reporter antibody determined in step 4.

In some embodiments, in step 2 the amount of the microsphere complex and the amount of the reporter antibody are concomitantly contacted with the sample.

In some embodiments, the method for determining norovirus-reactive antibodies comprises the steps of:
Step 1: providing a kit, including an amount of a microsphere complex as described above and an amount of a reporter antibody as described above, wherein the reporter antibody binds to the norovirus VLP of the microsphere complex,
Step 2.1: contacting the amount of the microsphere complex of step 1 with the sample to allow binding of norovirus-reactive antibodies in the sample to the norovirus VLPs coupled to the microspheres in the microsphere complex,
Step 2.2: contacting the amount of the reporter antibody with the microsphere complex and the sample of step 2.1 to allow binding of the reporter antibody to the norovirus VLPs coupled to the microspheres in the microsphere complex, and
Step 3: detecting a signal from the reporter antibody bound to the norovirus VLPs in step 2.2,
and wherein the method optionally comprises the further steps of:
Step 4: determining the presence and/or amount of the reporter antibody from the signal of step 3, and
Step 5: determining the presence and/or amount of norovirus-reactive antibodies in the sample from the presence and/or amount of the reporter antibody determined in step 4.

In some embodiments, the method for determining norovirus-reactive antibodies comprises the steps of:
Step 1: providing a kit, including an amount of a microsphere complex as described above and an amount of a reporter antibody as described above, wherein the reporter antibody binds to the norovirus VLP of the microsphere complex,
Step 2.1: contacting the amount of the microsphere complex of step 1 with the sample to allow binding of norovirus-reactive antibodies in the sample to the norovirus VLPs coupled to the microspheres in the microsphere complex,
Step 2.2: contacting the amount of the reporter antibody with the microsphere complex and the sample of step 2.1 to allow binding of the reporter antibody to the norovirus VLPs coupled to the microspheres in the microsphere complex,
Step 2.3: contacting the amount of reporter antibody, the amount of microsphere complex, and the sample of step 2.2 with an amount of a secondary reporter antibody to allow binding of the secondary reporter antibody to the constant region of the reporter antibody, and
Step 3: detecting a signal from the secondary reporter antibody bound to the reporter antibody in step 2.3, wherein the reporter antibody is bound to the norovirus VLPs in step 2.2.
and wherein the method optionally comprises the further steps of:
Step 4: determining the presence and/or amount of the reporter antibody from the signal of step 3, and
Step 5: determining the presence and/or amount of norovirus-reactive antibodies in the sample from the presence and/or amount of the reporter antibody determined in step 4.

In some embodiments, the norovirus VLP is a GII.4/Sydney VLP.

In some embodiments, the signal in step 3 is resulting from the detectable label the reporter antibody is attached to.

In some embodiments, contacting in step 2.1 is carried out for about 5 to about 23 hours, e.g., in some embodiments, for about 8 to about 21 hours, more preferably for about 16 hours. In some embodiments, contacting in step 2.1 is carried out at a temperature of about 2 to about 30° C., preferably at a temperature of about 4° C.

In some embodiments, contacting in step 2.2 is carried out for about 10 to about 90 minutes, preferably for about 60 minutes. In some embodiments, contacting in step 2.2 is carried out at about 22° C.

In some embodiments, contacting in step 2.3 is carried out for about 10 to about 90 minutes, preferably for about 60 minutes. In some embodiments, contacting in step 2.3 is carried out at about 22° C.

Method for Concomitant Determination of the Presence and/or Amount of Antibodies Reactive to Different Noroviruses (Multiplex Assay Set-Up)

The method for concomitant determination of the presence and/or amount of antibodies reactive to different noroviruses in a sample from a subject comprises the steps of:
Step 1: providing a kit, including an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies as described above,
wherein the first reporter antibody binds to the first norovirus VLP and does not bind to the second norovirus virus like particle, and
wherein the second reporter antibody binds to the second norovirus VLP and does not bind to the first norovirus VLP;
Step 2: contacting the amount of the at least two microsphere complexes with the sample to allow binding of the norovirus-reactive antibodies in the sample to the first and/or the second norovirus VLPs while competing with the at least two reporter antibodies;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the reporter antibody bound to the norovirus VLPs of the at least one microsphere upon in step 2 irradiation with a second light source;
Step 4: repeating step 3 until at least 30 microspheres coupled to the same norovirus VLP are identified; and
Step 5: summarizing the detected signal from the reporter antibody in step 3 for all identified microspheres coupled to the same norovirus VLP, wherein the summarized signal is indicative for the presence and/or amount of norovirus-reactive antibodies in the sample,
and wherein the method optionally comprises the further steps of:
Step 6: determining the presence and/or amount of the reporter antibody from the summarized signal of step 5, and
Step 7: determining the presence and/or amount of norovirus-reactive antibodies in the sample from the presence and/or amount of the reporter antibody determined in step 6.

In some embodiments, in step 2 the amount of the at least two microsphere complexes and the amount of the at least two reporter antibodies are concomitantly contacted with the sample.

In some embodiments, the method comprises the steps of:
Step 1: providing a kit, including an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies as described above,
wherein the first reporter antibody binds to the first norovirus VLP and does not bind to the second norovirus virus like particle, and
wherein the second reporter antibody binds to the second norovirus VLP and does not bind to the first norovirus VLP;
Step 2.1: contacting the amount of the at least two microsphere complexes with the sample to allow binding of the norovirus-reactive antibodies in the sample to the first and/or the second norovirus VLPs;
Step 2.2: contacting the amount of the at least two reporter antibodies with the at least two microsphere complexes and the sample of step 2.1 to allow binding of the at least two reporter antibodies to the norovirus VLPs coupled to the microspheres;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the reporter antibody bound to the norovirus VLPs of the at least one microsphere in step 2.2 upon irradiation with a second light source;
Step 4: repeating step 3 until at least 30 microspheres coupled to the same norovirus VLP are identified; and
Step 5: summarizing the detected signal from the reporter antibody in step 3 for all identified microspheres coupled to the same norovirus VLP, wherein the summarized signal is indicative for the presence and/or amount of norovirus-reactive antibodies in the sample,
and wherein the method optionally comprises the further steps of:
Step 6: determining the presence and/or amount of the reporter antibody from the summarized signal of step 5, and
Step 7: determining the presence and/or amount of norovirus-reactive antibodies in the sample from the presence and/or amount of the reporter antibody determined in step 6.

In some embodiments, the method comprises the steps of:
Step 1: providing a kit, including an amount of at least two microsphere complexes as described above,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies as described above,
wherein the first reporter antibody binds to the first norovirus VLP and does not bind to the second norovirus virus like particle, and
wherein the second reporter antibody binds to the second norovirus VLP and does not bind to the first norovirus VLP;
Step 2.1: contacting the amount of the at least two microsphere complexes with the sample to allow binding of the norovirus-reactive antibodies in the sample to the first and/or the second norovirus VLPs;
Step 2.2: contacting the amount of the at least two reporter antibodies with the at least two microsphere complexes and the sample of step 2.1 to allow binding of the at least two reporter antibodies to the norovirus VLPs coupled to the microspheres;
Step 2.3: contacting the amount of the at least two reporter antibodies, the amount of the at least two microsphere complexes and the sample of step 2.2 with an amount of a secondary reporter antibody to allow binding of the secondary reporter antibody to the constant region of the at least two reporter antibodies;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the secondary reporter antibody bound to the reporter antibody bound to the norovirus VLPs of the at least one microsphere in step 2.3 upon irradiation with a second light source;
Step 4: repeating step 3 until at least 30 microspheres coupled to the same norovirus VLP are identified; and
Step 5: summarizing the detected signal from the secondary reporter antibody in step 3 for all identified microspheres coupled to the same norovirus VLP, wherein the summarized signal is indicative for the presence and/or amount of norovirus-reactive antibodies in the sample,
and wherein the method optionally comprises the further steps of:
Step 6: determining the presence and/or amount of the reporter antibody from the summarized signal of step 5, and
Step 7: determining the presence and/or amount of norovirus-reactive antibodies in the sample from the presence and/or amount of the reporter antibody determined in step 6.

In some embodiments, the kit in step 1 provides an amount of two microsphere complexes and an amount of two reporter antibodies. In some embodiments, the first microsphere complex comprises a first microsphere coupled to a GI.1 VLP and wherein the second microsphere complex comprises a second microsphere coupled to a GII.4/Consensus VLP. For the GI.1 VLP and the GII.4/Consensus VLP, reference is also made to Table 1.

In some embodiments, the signal in step 3 is resulting from the detectable label the reporter antibody is attached to.

In some embodiments, contacting in step 2.1 is carried out for about 5 to about 23 hours, preferably for about 8 to about 21 hours, more preferably for about 16 hours. In some embodiments, contacting in step 2.1 is carried out at a temperature of about 2 to about 30° C., preferably at a temperature of about 4° C.

In some embodiments, contacting in step 2.2 is carried out for about 10 to about 90 minutes, preferably for about 60 minutes. In some embodiments, contacting in step 2.2 is carried out at about 22° C.

In some embodiments, contacting in step 2.3 is carried out for about 10 to about 90 minutes, preferably for about 60 minutes. In some embodiments, contacting in step 2.3 is carried out at about 22° C.

In some embodiments, step 4 is repeated until at least 35, at least 40, at least 45, or at least 50 microspheres coupled to the same norovirus VLP are identified.

### Method for Diagnosing a Norovirus Infection in a Subject

The present disclosure is further directed to a method for diagnosing a norovirus infection in a subject. Within the meaning of the disclosure, the method for diagnosing is an in vitro method. Regarding the microsphere complex, the detection system, the reporter, secondary reporter and detection antibody, the subject and sample, as well as the methods for determining norovirus-reactive antibodies, reference is made to the respective chapters above. In addition, certain specific embodiments are also outlined in this section and shall, however, not be taken as limiting. Any embodiments from this section can be combined with any of the embodiments from the previous or following sections.

The in vitro method for diagnosing a norovirus infection in a subject comprises the steps of:
Step 1: providing a sample from the subject outside the subject body,
Step 2: determining the amount of norovirus-reactive antibodies in the sample according to the methods for determining norovirus-reactive antibodies as described above, and
Step 3: determining infection by comparing the amount of norovirus-reactive antibodies to established amounts of norovirus-reactive antibodies in norovirus infected subjects.

In some embodiments the subject is a mammal, preferably the mammal is selected from the group consisting of mouse, primate, non-human primate, human, rabbit, cat, rat, horse, and sheep. In some embodiments the subject is a human.

In certain embodiments the sample is a blood sample, in particular a blood plasma or serum sample.

In certain embodiments the norovirus-reactive Abs are norovirus-neutralizing Abs and/or norovirus-blocking Abs.

In certain embodiments the norovirus infection is convalescent. In certain embodiments the norovirus infection is acute.

In other embodiments the subject is infected by at least two different noroviruses. The norovirus infections can be either acute or convalescent. The in vitro method for diagnosing a norovirus infection of the present application is capable of diagnosing the at least two different norovirus infections. Consequently, the in vitro method for diagnosing a norovirus infection of the present application is capable of determining whether a subject was infected with one or more noroviruses and by which noroviruses the subject was infected.

### Method for Determining Protection Against Norovirus Infection in a Subject

The present disclosure is further directed to a method for determining protection against a norovirus infection in a subject. Within the meaning of the disclosure, the method for determining protection is an in vitro method. Regarding the microsphere complex, the detection system, the reporter, secondary reporter and detection antibody, the subject and sample, as well as the methods for determining norovirus-reactive antibodies, reference is made to the respective chapters above. In addition, certain specific embodiments are also outlined in this section and shall, however, not be taken as limiting. Any embodiments from this section can be combined with any of the embodiments from the previous or following sections.

The in vitro method for determining protection against a norovirus infection in a subject comprises the steps of:
Step 1: providing a sample from the subject outside the subject body,
Step 2: determining the amount of norovirus-reactive antibodies in the sample according to the methods for determining norovirus-reactive antibodies as described above, and
Step 3: determining protection by comparing the amount of norovirus-reactive antibodies in step 2 to protective amounts of norovirus-reactive antibodies.

In some embodiments the subject is a mammal, preferably the mammal is selected from the group consisting of mouse, primate, non-human primate, human, rabbit, cat, rat, horse, and sheep. In some embodiments the subject is a human.

In other embodiments the subject is vaccinated with a norovirus vaccine. In some embodiments the subject is a human vaccinated with a norovirus vaccine.

In certain embodiments the sample is a blood sample, in particular a blood plasma or serum sample.

In certain embodiments the norovirus-reactive Abs are norovirus-neutralizing Abs and/or norovirus-blocking Abs.

In embodiments wherein protection against norovirus infection is determined with a competitive microsphere immunoassay set-up, it is preferred that the reporter or at least one reporter Ab is a norovirus-neutralizing and/or norovirus-blocking Ab.

The in vitro method for determining protection against a norovirus infection of the present application is capable of determining protection against one or more different norovirus infections.

### Method for Preventing Norovirus Infection

The present disclosure is further directed to a method for preventing norovirus infection in a human subject, the method comprising the steps of:
Step 1: obtaining a sample from the human subject,
Step 2: determining the amount of norovirus-reactive antibodies in the sample from the human subject as described above under the section “Method for determining norovirus-reactive antibodies”,
Step 3: determining whether the human subject has an amount of norovirus-reactive antibodies to confer protection by comparing the amount of norovirus-reactive antibodies determined in step 2 to the antibody correlate of protection against norovirus infection in human subjects, and
Step 4: administering to the human subject a norovirus vaccine if the human subject has an amount of norovirus-reactive antibodies that is lower than the antibody correlate of protection against norovirus infection in human subjects.

“Confer protection” within that context means that the amount of norovirus-reactive antibodies present in the human subject is sufficient to prevent the human subject from a norovirus infection.

“Antibody correlate of protection” within that context means a certain amount of norovirus-reactive antibodies that has been determined to confer protection against norovirus infection. An antibody correlate of protection can be, for instance, determined from suitable animal models or by monitoring protection of human subjects against norovirus infection, for instance, after being vaccinated with a norovirus vaccine.

In some embodiments, the norovirus infection is a symptomatic infection.

### Method for Assaying the Presence of a Norovirus Infection

The present disclosure is further directed to a method for assaying the presence of a norovirus infection in a subject comprising the steps of:
Step 1: obtaining a sample from the subject,
Step 2: determining the amount of norovirus-reactive antibodies in the sample as described above under the section “Method for determining norovirus-reactive antibodies”, and
Step 3: determining the presence of a norovirus infection by comparing said amount of norovirus-reactive antibodies to established amounts of norovirus-reactive antibodies in norovirus infected subjects.

### Kit for Determining Norovirus-Reactive Antibodies in a Sample

The present disclosure is further directed to a kit. Regarding the microsphere complex, the detection system, the reporter, secondary reporter and detection antibody, as well as the sample, reference is made to the respective chapters above. In addition, certain specific embodiments are also outlined in this section and shall, however, not be taken as limiting. Any embodiments from this section can be combined with any of the embodiments from the previous sections.

In one embodiment, the kit comprises an amount of at least one microsphere complex as described above and optionally an amount of a detection antibody as described above.

In other embodiments, the kit comprises an amount at least one microsphere complex as described above and an amount of at least one reporter antibody as described above, wherein the reporter antibody binds to the norovirus VLP of the at least one microsphere complex. In some embodiments, the kit additionally comprises an amount of a secondary reporter antibody as described above, wherein the secondary reporter antibody binds to the reporter antibody.

The kit may further contain a suitable container for the mixture of the components of the kit. The kit may further contain a manual with instructions.

### EXAMPLES

The following Examples are included to demonstrate certain aspects and embodiments of the disclosure as described in the claims. It should be appreciated by those of skill in the art, however, that the following description is illustrative only and should not be taken in any way as a restriction of the disclosure.

### Example 1: Production of Norovirus Virus Like Particles (VLPs)

VLPs consisting of the major viral capsid protein, VP1, were produced as described in Haynes et al. (Viruses 2019, 11, 392, doi:10.3390/v11050392), Parra et al. (Vaccine 2012, 30(24): 3580-3586, doi: 10.1016/j.vaccine.2012.03.050) and WO 2010/017542. An overview of Norovirus VLPs as used herein, including corresponding strains and GenBank accession numbers is given in Table 1.

For production of the GII.4/Den Haag, GII.4/Yerseke, GII.4/New Orleans, GII.4/Sydney, as well as GII.7 VLPs, VP1 amino acid sequences obtained from GenBank (Table 1) were used to synthesize a mammalian codon optimized nucleotide gene sequence for each particular VP1 protein (synthesized at ATUM, Newark, USA). Restriction sites were engineered onto the ends of the synthetic genes to facilitate cloning into the AdEasy Adenoviral Vector System Cloning kit from Agilent that was used to produce the recombinant adenovirus clones. The recombinant adenoviruses were used to infect Vero cells at a multiplicity of infection of 300, and cultures were harvested after 4 days. The supernatant was removed from cells and the adherent cells were treated with a phosphate buffered saline (PBS) and 0.1% Tween solution for 5 min with rocking at room temperature to lyse cells. The lysate was clarified by centrifugation (400×g) and filtered through a 0.45 μm syringe filter and then spun into a 40% sucrose cushion in an ultracentrifuge (100,000×g). Purity of VLPs was assessed by SDS-PAGE and concentration determined by BCA assay. The VLPs were frozen at −80° C. in a 40% sucrose/PBS buffer. Alternatively, the VLPs that were expressed in mammalian cells, can also be expressed in baculovirus cells as described below.

For production of the remaining VLPs listed in Table 1, a baculovirus expression system was used. The GII.4 consensus norovirus VLP amino acid sequence was designed by aligning the following human norovirus GII.4 major capsid protein sequences and determining the “consensus” amino acid residues at each position: Houston/TCH186/2002/US (GenBank ABY27560.1), DenHaag89/2006/NL (GenBank ABL74395.1), and Yerseke38/2006/NL (GenBank ABL74391.1). At those amino acid positions where a different residue was found in each sequence, the amino acid residue found in the Yerseke38 sequence was chosen because fewer substitutions were needed to achieve consensus among the three strains. Synthetic DNA fragments encoding the corresponding VP1 sequences with codon optimization for Spodoptera frugiperda Sf9 cells were synthesized by GeneArt (Regensburg, Germany) and engineered into a recombinant baculovirus for expression of VLPs. Sf9 cells were infected at low multiplicity of infection (MOI) and supernatant was harvested ˜5 days post infection. Following production, VLPs were purified using multiple orthogonal chromatography operations.

TABLE 1
Norovirus VLPs as used herein, including corresponding norovirus strains
and GenBank accession numbers for corresponding VP1 sequences.

| | | | SEQ ID |
|---|---|---|---|
| VLP Abbreviation | Origin Norovirus | GenBank | NO: |
| GI.1 VLP | Hu/GI.1/Norwalk/1968/US | AAB50466.2 | 1 |
| GI.2 VLP | Hu/GI.2/Jingzhou/2013401/CHN | AGX01092.1 | 2 |
| GI.3 VLP | Hu/GI.3/JKPG_883/SWE/2007 | ACX33983.1 | 3 |
| GI.4 VLP | Hu/GI.4/1643/2008/US | ACV41096.1 | 4 |
| GI.5 VLP | Hu/GI.5/Siklos-HUN5407/2013/HUN | AHW99832.1 | 5 |
| GI.6 VLP | Hu/GI.6/TCH-099/USA/2003 | AGT62521.1 | 6 |
| GI.7 VLP | Hu/GI.7/Providence191/2010/USA | AFC89659.1 | 7 |
| GII.1 VLP | Hu/GII.1/Ascension208/2010/USA | AFA55174.1 | 8 |
| GII.2 VLP | Hu/GII.2/OH10026/2010/JP | BAL60847.1 | 9 |
| GII.3 VLP | Hu/GII.3/NIHIC8.1/2011/USA | AGI17594.1 | 10 |
| GII.4/Consensus VLP | Hu/GII.4/Houston/TCH186/2002/US | N/A | 11 |
| | Hu/GII.4/DenHaag89/2006/NL | | |
| | Hu/GII.4/Yerseke38/2006/NL | | |
| GII.4/Sydney VLP | Hu/GII.4/Sydney/NSW0514/2012/AU | AFV08795.1 | 12 |
| GII.4/Yerseke VLP | Hu/GII.4/Yerseke38/2006/NL | ABL74391.1 | 13 |
| GII.4/New Orleans VLP | Hu/GII.4/New Orleans1805/2009/USA | ADD10375.1 | 14 |
| GII.4/Den Haag VLP | Hu/GII.4/DenHaag89/2006/NL | ABL74395.1 | 15 |
| GII.4/Houston VLP | Hu/GII.4/Houston/TCH186/2002/US | ABY27560.1 | 16 |
| GII.6 VLP | Hu/GII.6/Ehime120246/2012/JP | BAN16289.1 | 17 |
| GII.7 VLP | Hu/GII.7/NSW088L/2007/AUS | ACX85809.1 | 18 |
| GII.12 VLP | Hu/GII.12 strain E5152 | AJP13623.1 | 19 |
| GII.17/1978 VLP | Hu/GII.17/C142/GF/1978 | AFN06732.1 | 20 |
| GII.17/2014 VLP | Hu/GII.17/JP/2014/Nagano7-1 | BAR47616.1 | 21 |
| GII.17/2015 VLP | Hu/GII.17/HKG/2015/CUHK-NS-513 | AKB94548.1 | 22 |

### Example 2: Coupling of Norovirus VLPs to Microspheres

Microspheres used for coupling were MagPlex® microspheres (Luminex Corporation, Austin, Texas). MagPlex® microspheres are superparamagnetic polystyrene microspheres with surface carboxyl groups. The microspheres were delivered in a volume of 4 to 4.1 mL with an average concentration of 1.2 to 1.3×10⁷ microspheres per mL (microspheres/mL). MagPlex® microspheres are available in several unique regions, i.e. the microspheres comprise one or more fluorescent dyes having a defined emission signal (the detectable label) in order to distinguish the microspheres from microspheres of other unique regions. As the coupling mechanism involving the surface carboxyl groups is independent of the detectable label of the microspheres, MagPlex® microspheres of different unique regions may be exchanged according to variations in experimental set-ups.

Different microspheres comprising one or more fluorescent dyes having a specific emission signal (different unique regions/detectable labels) were applied for coupling of the different norovirus VLPs as described under Example 1 to provide the possibility to distinguish the microspheres according to their coupled VLPs when analyzed within one sample (capability to multi-plex). For example, GI.1 VLPs were coupled to MagPlex® microspheres of region 14 (Catalog number MC10014-04, Product Lot. B65330), GII.4/Sydney VLPs were coupled to MagPlex® microspheres of region 25 (Catalog number MC10025-04; Product Lot. B67632) and GII.4/Consensus VLP were coupled to MagPlex® microspheres of region 47 (Catalog number MC10047-04, Product Lot. B69911).

### General Procedure for Coupling of Norovirus Antigens to Microspheres

The uncoupled stocks of MagPlex® microsphere suspensions (1.2 to 1.3×10⁷ microspheres/mL, Luminex Corporation, Austin, Texas) were resuspended by vortexing (30 sec) and up to 5×10⁶ microspheres of each stock were transferred to 1.5 mL microcentrifuge tubes and placed into a 1.5 mL tubes magnetic separator (Life Technologies, Cat. No. 44578578). Separation of the microspheres from the suspension occurred for 30-60 sec. Supernatant was carefully removed without disrupting the microsphere pellet while the tubes were still positioned in the magnetic separator. Afterwards, the tubes were removed from the magnetic separator and the microspheres were resuspended in 100 μL distilled H₂O (dH₂O) by vortexing and sonication for approximately 20 sec. The tubes were again placed into the magnetic separator and separation of the microspheres from the suspension occurred for 30-60 sec. Supernatant was removed without disrupting the microsphere pellet while the tubes were still positioned in the magnetic separator. The microspheres were resuspended in 80 μl of activation buffer (0.1 M sodium phosphate (monobasic) pH 6.5) and mixed by vortexing and sonication for 20 sec. Then, 10 μL of 50 mg/mL N-hydroxysulfosuccinimide (Sulfo-NHS; 2 mg of Sulfo-NHS in 40 μL of dH₂O; Thermo Fisher Scientific, Cat. No. A39269, Lot. No. TJ272218) were added to each microsphere tube and gentle mixing was carried out by vortexing (5 sec). Further, 10 μL of 50 mg/mL 1-Ethyl-3-(3-dimethylaminopropyl)carbodiimide (EDC; 1 mg EDC in 20 μL of dH₂O; Thermo Fisher Scientific, Cat. No. A35391, Lot. No. UD277513) were added to each microsphere tube and gentle mixing was carried out by vortexing (5 sec). Samples were incubated for 20 minutes at room temperature under rotation. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator. The microspheres were washed twice with 300 μL 1× Phosphate Buffered Saline, PBS (sterile), vortexed and sonicated for approximately 20 sec.

500 μL of VLPs (diluted in 1×PBS) were transferred to the respective 1.5 mL tube containing the activated microspheres to result in a ratio of 1.2 μg VLP per 10⁶ microspheres in a total volume of 500 μL. For coupling, samples were incubated for 2 hours under rotation at room temperature. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 500 μL 0.05% (v/v) Tween-20 in PBS pH 7.4 for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 2.0 mL of 20 mM histidine buffer.

The number of microspheres recovered after the coupling reaction was determined using an automated cell counter (Countess II, Thermo Fisher Scientific, Cat. No. AMQAX1000) by correlating the determined “dead cells” concentration provided by the cell counter to the microspheres. The coupled microspheres were stored at 2-8° C. in the dark.

### Optimization of Coupling Conditions

The coupling efficiency, as well as the integrity of the antigen after the coupling procedure is dependent on various factors such as the buffer and antigen amount used. Optimization of the coupling procedure is important in order to ensure that the three-dimensional structure of the antigen is not disturbed. The buffer conditions may vary dependent on the type of antigen used.

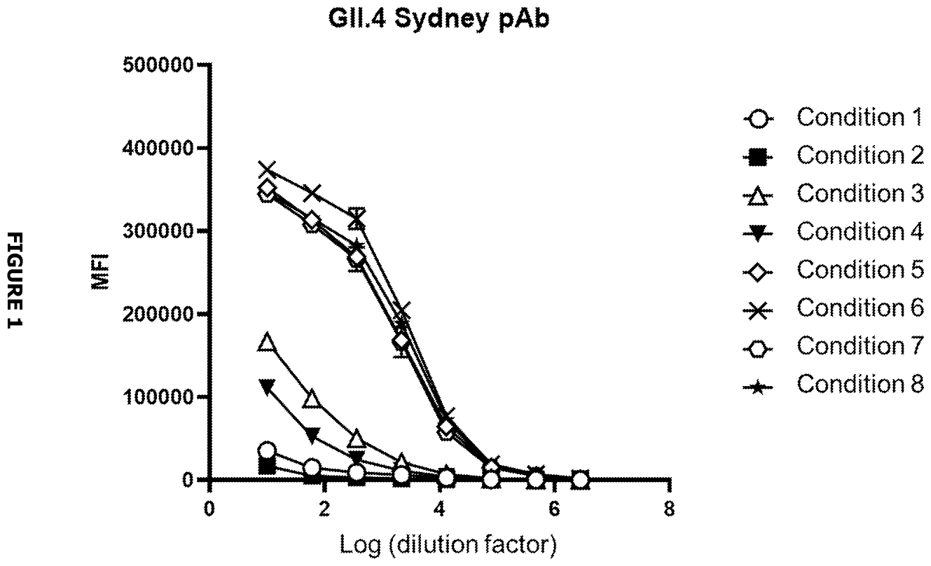

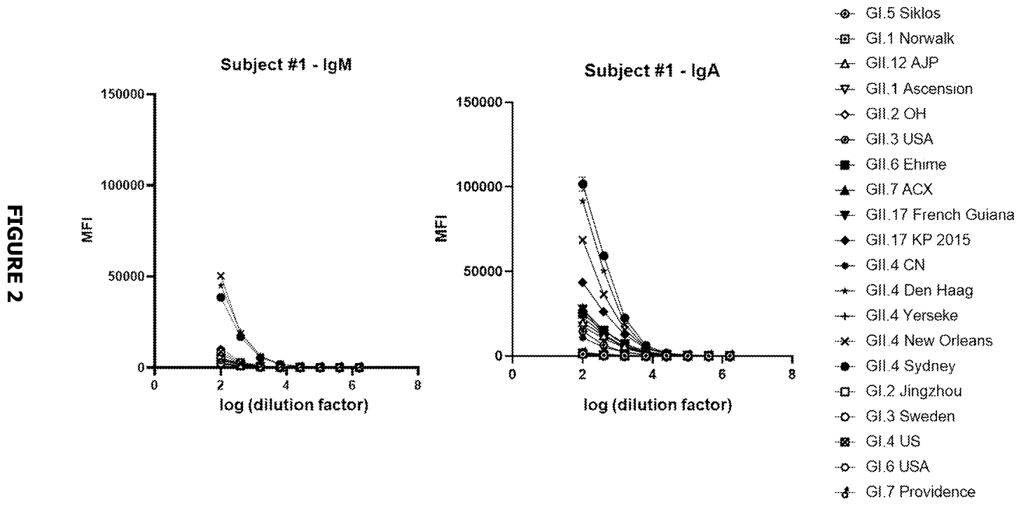

Therefore, the general procedure described above was carried out, but with different coupling buffers, amounts of NHS and EDC, as well as amounts of VLP. In addition to coupling in 1×PBS, coupling also carried out in 50 mM 2-(N-morpholino)ethanesulfonic acid (MES) at pH 5, 6, and 7. Further, In addition to a volume of 10 μL NHS and EDC each, also a volume of 25 μL was tested. Moreover, the amount of VLPs per 5×10⁶ microspheres was varied (3, 10, 30 μg VLP). Besides these modifications, coupling was carried out as described above. The coupling efficacy was evaluated by the non-competitive assay set-up as described under Example 3 using suitable polyclonal antibodies and corresponding detection antibodies. Optimization is exemplarily shown for coupling of GII.4/Sydney (Table 2 and FIG. 1).

TABLE 2
Coupling conditions for GII.4/Sydney and EC50 values determined
using a polyclonal anti-GII.4/Sydney antibody.

| Condition | μL NHS/EDC | μg VLP | Buffer | EC₅₀ |
|---|---|---|---|---|
| 1 | 10 | 30 | 50 mM MES pH 5 | 0.09782 |
| 2 | 25 | 3 | 50 mM MES pH 5 | 0.1465 |
| 3 | 25 | 30 | 50 mM MES pH 6 | 15.6 |
| 4 | 10 | 10 | 50 mM MES pH 6 | 3.63 |
| 5 | 10 | 3 | 50 mM MES pH 7 | 2908 |
| 6 | 25 | 10 | 50 mM MES pH 7 | 4526 |
| 7 | 10 | 3 | 1xPBS pH 7.4 | 2714 |
| 8 | 25 | 10 | 1xPBS pH 7.4 | 3772 |

Coupling of the GII.4/Sydney VLP was only efficient for conditions 5 to 8 indicating significant differences in coupling efficacies depending e.g. on the buffer pH.

For coupling of norovirus VLPs to microspheres as used in the Examples below, the general procedure as described above (in 1×PBS with 1.2 μg VLP and each 10 μL of EDC and Sulfo-NHS) was mainly carried out as this procedure resulted in efficient coupling for all VLPs.

### Example 3: Determination of Antibody Titers in a Non-Competitive Microsphere Immunoassay Set-Up

To determine total anti-norovirus antibody amounts in human samples, a non-competitive multiplex microsphere immunoassay set-up was developed. In brief, human serum samples were incubated with a mixture of different microspheres (“multiplex” set-up), wherein each microsphere was coupled to a different norovirus VLP to allow binding of the norovirus-reactive Abs in the sample to the corresponding norovirus VLPs. Afterwards, total IgG, IgA, and IgM amounts were determined using corresponding detection antibodies coupled to phycoerythrin (PE). “Total” within that context means that the non-competitive microsphere immunoassay detects essentially all or a major part of the Abs in the sample, which are capable of binding to a corresponding norovirus VLP.

This assay set-up allows evaluation and characterization of the complete acute and convalescent immune response after single or multiple norovirus infections or after vaccination against norovirus. “Complete” within that context means that the determined immune response is characterized by the determined “total” antibody titers or antibody amounts. By determining the immune status, natural infection and vaccination may be distinguished. In addition, progression of immune response and changes of immune status over time can be analyzed. The assay may be further suitable to determine whether antibody titers are protective or not by comparing to antibody titers from protected individuals. Moreover, the assay enables monitoring cross-reactive antibody responses over time after infection with a certain norovirus type or after vaccination. In addition, the assay allows evaluation of changes of antibody patterns after a second or further norovirus infection. As the assay is able to distinguish between isotype-specific responses (IgM, IgA, IgG), it is also possible to analyze which isotype may be the best indicator for infection or immune status after vaccination, or to determine if there is a temporal appearance of isotypes (e.g. IgM in general is the first indicator) after norovirus infection or vaccination. Moreover, the assay is suitable for characterization of the passive transfer of maternal antibodies to infants.

### General Method

Human test samples (e.g. human serum samples) and control serum sample (Bioreclamation, Cat-No. HUMANSRM1800041) were heat-inactivated in a 56±1° C. water bath for 30±1 min prior to testing. After heat-inactivation, samples were vortexed and placed on ice prior to dilution in assay buffer, i.e. phosphate buffer saline (PBS) with 1% bovine serum albumin (BSA). For preparation of assay buffer, BSA was diluted from a 10% stock (Thermo Fisher, Cat-No. 37525) in PBS (Gibco, Cat-No. 10010-023). Samples were diluted serially resulting in 100-, 400-, 1600-, 6400-, 25600-, 102400-, 409600-, and 1638400-fold dilutions. Each 100 μL per dilution were plated per well in duplicates into a 96-well plate (polystyrene solid black flat bottom microplate, Corning, Cat-No. 3915). Then, corresponding norovirus VLP-coupled microspheres were diluted and mixed by vortexing to result in a final concentration of 10-30 microspheres/μL for each norovirus VLP in assay buffer. Depending on the number of different microsphere types applied (e.g. duplex, triplex, or quadruplex set-up), the concentration of each microsphere type is adjusted. For instance, in a duplex set-up, a higher microsphere concentration can be used per microsphere type, whereas in a 20-plex set-up, the concentration per microsphere type is less, as the total amount of microspheres should not overcome a certain maximum. Afterwards, 50 μL/well of the microsphere mixture were added to the plate resulting in a total volume of 150 μL. The plate was sealed with a foil plate seal (Thermo Fisher, Cat-No. AB0558) and incubated for 21±3 hours at 2-8° C. Alternatively, the plate can be incubated for 90±5 min at room temperature on a plate shaker at 600 rpm.

In the next step, detection antibodies were added for detection. Suitable detection antibodies were goat anti-human pan-Ig antibody conjugated to phycoerythrin (PE) (Southern Biotech, Cat-No. 2010-09, Lot-No. C2117-SG98) reacting with human IgG, IgM, and IgA, and goat anti-human IgG, IgA, and IgM antibodies conjugated to PE (Southern Biotech, Cat-No. 2040-09, 2050-09, 2020-09, respectively). All antibodies were delivered at a stock concentration of 0.5 mg/mL in PBS containing 0.1% sodium azide and a stabilizer. Detection antibodies were diluted 1:25 (goat anti-human pan-Ig antibody) or 1:50 (goat anti-human IgG, IgA, and IgM antibodies) in assay buffer. After incubation, the plate was washed with wash buffer (PBS with 0.05% Tween-20) using a plate washer with a magnet (magnetic plate separator, BioTek, EL-406/405-TS). 50 μL of the diluted detection antibody were added per well and the plate was again sealed using a foil plate seal. The plate was incubated for 60±5 min at room temperature on a plate shaker set to 600 rpm. After incubation, the plate was again washed using wash buffer as described above. Finally, 95 μL/well of sheath fluid (Luminex Corp., Cat-No. 40-50000) or 100 μL/well assay buffer were added, the plate was again sealed with a foil plate seal and shaken at 600 rpm until analysis. Sheath fluid is preferable for higher-plex set-ups as it helps reducing clumping of the microspheres. Alternatively, the plate with sheath fluid may be stored at 2-8° C. overnight for analysis on the following day. If the plate was stored at 2-8° C., the plate was shaken at room temperature at 600 rpm for at least 30 min prior to analysis.

The plate was analyzed using a FlexMap 3D Luminex Plate Reader with xPONENT 4.2 software (Luminex Corp, FM3D), setting the microsphere count to 50 for each of the different microspheres and sample volume to 50 μL per well. Further, the instrument acquisition settings were 30 sec timeout, gating was set at 7500 to 15000, and the reported gain was set to enhanced PMT (high). Recorded median fluorescence intensity (MFI) data were averaged from the duplicates and further analyzed using Prism (version 7.02, GraphPad). Averaged MFI values were plotted on the y-axis against corresponding log 10-transformed serum dilutions on the x-axis for each norovirus VLP. A 4-parameter curve fit analysis was performed. The resulting 50% effective concentration (EC₅₀) was interpolated from the curves and reported as sample titer.

### 20-Plex Assay Set-Up

Human serum samples were analyzed in a 20-plex assay set-up. Norovirus VLPs used in the 20-plex assay set-up were GI.1, GI.2, GI.3, GI.4, GI.5, GI.6, GI.7, GII.1, GII.2, GII.3, GII.4/Consensus, GII.4/Den Haag, GII.4/Yerseke, GII.4/New Orleans, GII.4/Sydney, GII.6, GII.7, GII.12, GII.17/1978, and GII.17/2015 (cf. Table 1). VLPs were prepared and coupled to the microspheres as described under Examples 1 and 2. The 20-plex assay set-up carried out as described above for the general method. For the 20-plex assay set-up, 10 microspheres/μL of each microsphere coupled to a different norovirus VLP were applied, VLPs and serum dilutions were incubated for 21±3 hours at 2-8° C., and 95 μL/well of sheath fluid (Luminex Corp., Cat-No. 40-50000) were added previous to analysis. IgG, IgA, and IgM were analyzed using goat anti-human IgG, IgA, and IgM antibodies as described above. Lower limits of quantification (LLOQ) for the assay were determined to be 59, 61 and 11 for IgG, IgA and IgM.

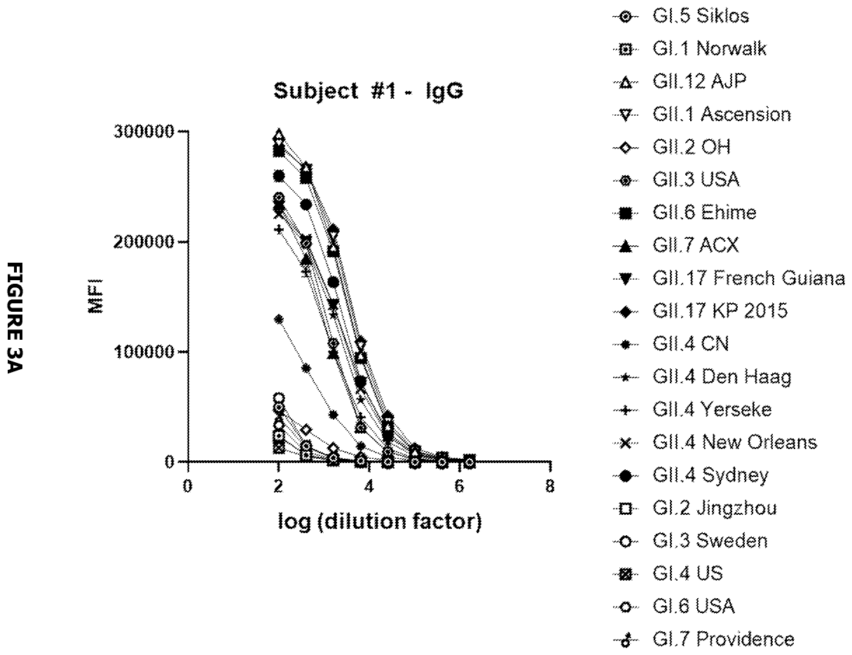

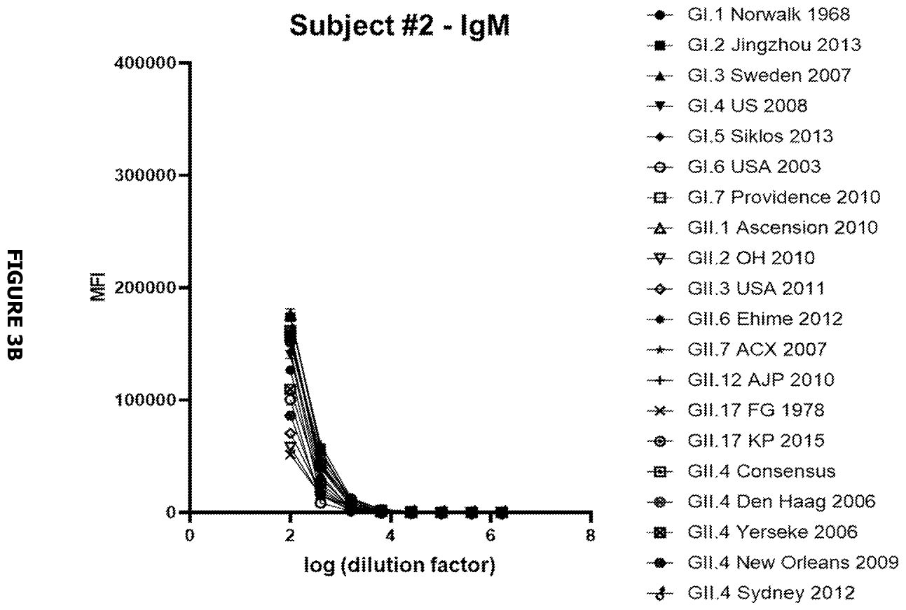

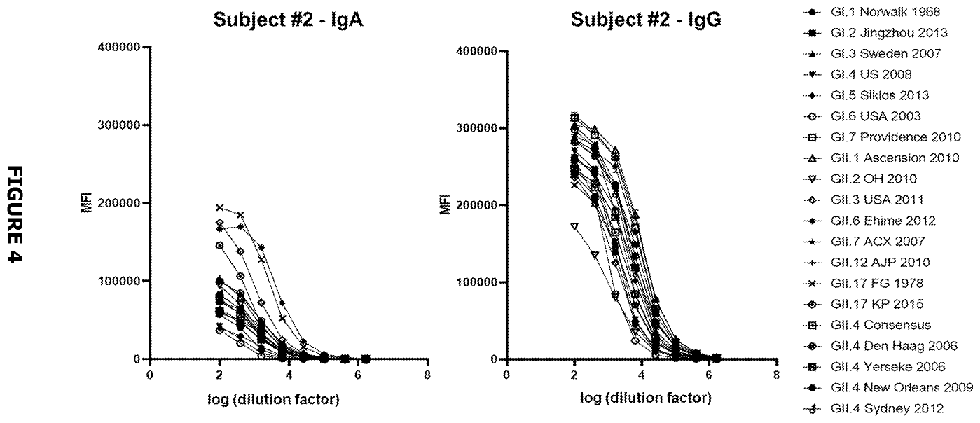

As a plateau was not reached with serum samples of subjects #1 and #2 (FIGS. 2 to 4), the top of the curves were constrained to 300,000 MFI for IgG and IgM and 200,000 MFI for IgA, respectively, in order to allow for a good 4 parameter fit. EC₅₀ values are exemplarily presented for the sample of subject #1 in Table 3. The exact exposure pattern of subjects #1 and #2 to norovirus was not known prior to testing.

TABLE 3
IgG, IgM, and IgA antibody titers in the sample from subject #1 against different norovirus
VLPs reported as EC₅₀ values as measured by the 20-plex non-competitive assay set-up.

| | | | | | | | | | GII.17/ | GII.17/ |
|---|---|---|---|---|---|---|---|---|---|---|
| | GI.5 | GI.1 | GII.12 | GII.1 | GII.2 | GII.3 | GII.6 | GII.7 | 1978 | 2015 |
| IgG | 19.16 | 8.987 | 3020 | 3406 | 3.19 | 778.3 | 2829 | 647.6 | 1218 | 3692 |
| IgA | 0.06302 | 0.2191 | 0.8913 | 0.7211 | 0.02594 | 2.199 | 1.945 | 6.975 | 2.991 | 8.371 |
| IgM | 0.6875 | 0.04978 | 1.319 | 0.1229 | 0.1381 | 0.1921 | 0.2058 | 0.01332 | 0.3516 | 0.1518 |

| | GII.4/ | GII.4/Den | GII.4/ | GII.4/New | GII.4/ | | | | | |
|---|---|---|---|---|---|---|---|---|---|---|
| | Consensus | Haag | Yerseke | Orleans | Sydney | GI.2 | GI.3 | GI.4 | GI.6 | GI.7 |
| IgG | 69.02 | 983.1 | 547.3 | 1178 | 1824 | 7.563 | 28.14 | 1.437 | 11.98 | 11.62 |
| IgA | 0.6613 | 83.07 | 2.096 | 37.95 | 111.6 | 0.1221 | 0.6245 | 0.1154 | 0.01819 | 0.1636 |
| IgM | 2.136 | 8.656 | 0.05088 | 14.13 | 6.263 | 0.2039 | 0.986 | 1.864 | 0.1014 | 0.8921 |

In summary, the 20-plex assay set-up enabled reliable measurement of antibody titers for isotypes G, M, and A against 20 different norovirus VLPs. For the majority of VLPs for samples from both subjects #1 and #2, IgG titers were the highest, followed by IgA and IgM titers. The 20-plex assay set-up showed good inter- and intra-assay precision with coefficient of variation (CV) below 17% for each isotype and good repeatability for each isotype with CV in the range of 2-5%.

To further evaluate if the 20-plex assay is suitable to follow changes in immune status over time and to evaluate maternal antibodies, as well as norovirus infection patterns in infants, serum samples from infants taken 2, 3, and 12 months after birth were analyzed.

Subject #A had a pattern expected for a child with no norovirus infection during the first 12 months (Table 4). The IgG titers at 2 months constitute maternal antibodies as these titers decay over time (see months 3 and 12). In addition, no or only very low IgA or IgM titers were detected, as those are not transferred maternally. For all VLPs, the titers decay until month 12 and none showed a clear increase that would be indicative of an infection or exposure to norovirus.

TABLE 4
IgG, IgM, and IgA antibody titers (EC₅₀) in samples from subject #A against different norovirus VLPs as
measured by the 20-plex non-competitive assay set-up. Samples were taken at 2, 3, and 12 months after birth.

| Timepoint | | | | | | | | | | | |
|---|---|---|---|---|---|---|---|---|---|---|---|
| (months) | Ig | GI.1 | GI.2 | GI.3 | GI.4 | GI.5 | GI.6 | GI.7 | GII.1 | GII.2 | GII.3 |
| 2 | IgA | 0 | 0 | 0 | 0 | 0 | 0 | 0 | 0 | 0 | 0 |
| 3 | IgA | 0 | 0 | 1 | 0 | 0 | 0 | 3 | 0 | 0 | 0 |
| 12 | IgA | 0 | 0 | 0 | 1 | 0 | 0 | 0 | 1 | 0 | 0 |
| 2 | IgG | 215 | 224 | 191 | 137 | 260 | 30 | 220 | 113 | 184 | 111 |
| 3 | IgG | 58 | 67 | 46 | 37 | 80 | 8 | 52 | 30 | 62 | 29 |
| 12 | IgG | 15 | 9 | 4 | 8 | 10 | 5 | 12 | 6 | 8 | 8 |
| 2 | IgM | 0 | 0 | 2 | 1 | 0 | 0 | 0 | 5 | 2 | 3 |
| 3 | IgM | 0 | 0 | 3 | 1 | 0 | 0 | 2 | 3 | 0 | 7 |
| 12 | IgM | 1 | 0 | 0 | 3 | 1 | 1 | 5 | 7 | 1 | 6 |

| Timepoint | | | | | GII.17/ | GII.17/ | GII.4/ | GII.4/Den | GII.4/ | GII.4/New | GII.4 |
|---|---|---|---|---|---|---|---|---|---|---|---|
| (months) | Ig | GII.6 | GII.7 | GII.12 | 1978 | 2015 | Consensus | Haag | Yerseke | Orleans | Sydney |
| 2 | IgA | 0 | 0 | 0 | 0 | 0 | 0 | 0 | 0 | 0 | 0 |
| 3 | IgA | 0 | 0 | 0 | 0 | 0 | 0 | 0 | 0 | 0 | 0 |
| 12 | IgA | 0 | 0 | 0 | 0 | 1 | 0 | 0 | 0 | 0 | 0 |
| 2 | IgG | 2178 | 888 | 245 | 574 | 106 | 772 | 1888 | 614 | 467 | 1193 |
| 3 | IgG | 580 | 234 | 57 | 161 | 25 | 246 | 544 | 187 | 143 | 327 |
| 12 | IgG | 26 | 11 | 4 | 7 | 5 | 16 | 17 | 7 | 4 | 11 |
| 2 | IgM | 8 | 4 | 1 | 1 | 18 | 4 | 7 | 1 | 2 | 6 |
| 3 | IgM | 1 | 0 | 1 | 0 | 18 | 1 | 3 | 0 | 0 | 2 |
| 12 | IgM | 30 | 2 | 2 | 0 | 32 | 3 | 0 | 0 | 0 | 1 |

Subject #B also showed maternal antibodies (IgG) at 2 months reactive to all norovirus types examined (Table 5). As for Subject #A, IgG levels declined over the course of 12 months, except for IgG antibodies against GI.7, which increased to a titer of 2436. Such a pattern would be suggestive of an infection with a GI.7 norovirus, as IgG increases are limited to the GI.7 VLP.

TABLE 5
IgG, IgM, and IgA antibody titers (EC₅₀) in samples from subject #B against different norovirus VLPs as
measured by the 20-plex non-competitive assay set-up. Samples were taken at 2, 3, and 12 months after birth.

| Timepoint | | | | | | | | | | | |
|---|---|---|---|---|---|---|---|---|---|---|---|
| (months) | Ig | GI.1 | GI.2 | GI.3 | GI.4 | GI.5 | GI.6 | GI.7 | GII.1 | GII.2 | GII.3 |
| 2 | IgA | 14 | 0 | 0 | 1 | 0 | 0 | 1 | 1 | 0 | 1 |
| 3 | IgA | 2 | 0 | 0 | 0 | 0 | 0 | 0 | 0 | 0 | 0 |
| 12 | IgA | 1 | 0 | 0 | 0 | 0 | 0 | 10 | 0 | 0 | 0 |
| 2 | IgG | 416 | 417 | 147 | 116 | 186 | 29 | 184 | 71 | 64 | 41 |
| 3 | IgG | 414 | 197 | 64 | 50 | 86 | 13 | 84 | 34 | 35 | 24 |
| 12 | IgG | 217 | 27 | 82 | 8 | 39 | 15 | 2436 | 3 | 2 | 7 |
| 2 | IgM | 4 | 0 | 4 | 0 | 0 | 21 | 11 | 11 | 1 | 14 |
| 3 | IgM | 0 | 0 | 2 | 1 | 1 | 1 | 2 | 7 | 2 | 6 |
| 12 | IgM | 0 | 3 | 0 | 5 | 1 | 1 | 2 | 4 | 1 | 11 |

| Timepoint | | | | | GII.17/ | GII.17/ | GII.4/ | GII.4/Den | GII.4/ | GII.4/New | GII.4 |
|---|---|---|---|---|---|---|---|---|---|---|---|
| (months) | Ig | GII.6 | GII.7 | GII.12 | 1978 | 2015 | Consensus | Haag | Yerseke | Orleans | Sydney |
| 2 | IgA | 0 | 0 | 1 | 0 | 0 | 0 | 0 | 0 | 0 | 0 |
| 3 | IgA | 0 | 0 | 0 | 0 | 0 | 0 | 0 | 0 | 0 | 0 |
| 12 | IgA | 0 | 0 | 0 | 0 | 0 | 0 | 0 | 0 | 0 | 0 |
| 2 | IgG | 484 | 654 | 201 | 14 | 26 | 249 | 358 | 306 | 76 | 329 |
| 3 | IgG | 213 | 309 | 89 | 7 | 13 | 123 | 164 | 144 | 40 | 151 |
| 12 | IgG | 4 | 8 | 4 | 0 | 4 | 8 | 3 | 3 | 1 | 3 |
| 2 | IgM | 7 | 1 | 6 | 2 | 46 | 3 | 0 | 0 | 1 | 0 |
| 3 | IgM | 2 | 0 | 1 | 1 | 17 | 3 | 0 | 0 | 3 | 1 |
| 12 | IgM | 10 | 5 | 1 | 1 | 21 | 5 | 0 | 1 | 1 | 2 |

Further, pediatric serum samples from a birth cohort were evaluated with the 20-plex assay set-up. Children in the cohort were followed for acute gastroenteritis and were tested for norovirus infections when symptomatic disease was found. As testing revealed with which norovirus genotype a particular child was infected, the results of the 20-plex assay can be compared to the confirmed infections.

Subject #C was diagnosed with a GII.12 infection at month 9, and samples collected 4 months prior and 9 months post infection were tested (Table 6). Both IgG and IgA showed clear enhancement to GII.12 VLP following infection, confirming that the assay is capable of specifically identifying an infection by a particular norovirus genotype. There is evidence of maternal antibody at 5 months when testing for IgG titers, however these diminished for most VLPs by 18 months. IgG titers for GI.12 are very high (7624) at 18 months due to the infection.

TABLE 6
IgG, IgM, and IgA antibody titers (EC₅₀) in samples from subject #C against different norovirus VLPs
as measured by the 20-plex non-competitive assay set-up. Samples were taken at 5 and 18 months after birth.

| Timepoint | | | | | | | | | | | |
|---|---|---|---|---|---|---|---|---|---|---|---|
| (months) | Ig | GI.1 | GI.2 | GI.3 | GI.4 | GI.5 | GI.6 | GI.7 | GII.1 | GII.2 | GII.3 |
| 5 | IgA | 0 | 0 | 0 | 0 | 0 | 0 | 0 | 0 | 0 | 0 |
| 18 | IgA | 0 | 0 | 0 | 0 | 0 | 0 | 0 | 1 | 0 | 1 |
| 5 | IgG | 115 | 101 | 136 | 72 | 186 | 28 | 155 | 52 | 87 | 32 |
| 18 | IgG | 3 | 2 | 5 | 0 | 9 | 2 | 7 | 116 | 13 | 41 |
| 5 | IgM | 1 | 1 | 3 | 2 | 2 | 1 | 4 | 9 | 3 | 14 |
| 18 | IgM | 1 | 0 | 8 | 0 | 1 | 0 | 6 | 2 | 0 | 2 |

| Timepoint | | | | | GII. 17/ | GII.17/ | GII.4/ | GII.4/Den | GII.4/ | GII.4/New | GII.4 |
|---|---|---|---|---|---|---|---|---|---|---|---|
| (months) | Ig | GII.6 | GII.7 | GII.12 | 1978 | 2015 | Consensus | Haag | Yerseke | Orleans | Sydney |
| 5 | IgA | 0 | 0 | 0 | 0 | 0 | 0 | 0 | 0 | 0 | 0 |
| 18 | IgA | 0 | 0 | 998 | 0 | 1 | 0 | 0 | 0 | 0 | 0 |
| 5 | IgG | 122 | 125 | 152 | 14 | 19 | 14 | 33 | 15 | 21 | 8 |
| 18 | IgG | 18 | 56 | 7624 | 19 | 37 | 8 | 16 | 13 | 5 | 23 |
| 5 | IgM | 31 | 2 | 3 | 3 | 41 | 4 | 0 | 0 | 0 | 3 |
| 18 | IgM | 9 | 1 | 0 | 0 | 31 | 2 | 0 | 0 | 0 | 1 |

Subject #D was diagnosed with a GII.4 Sydney infection at approximately 6½ months of age, and samples collected 1½ months prior and 5½ months post infection were tested (Table 7). IgG titers against GII.4 Sydney increased markedly at month 12. In addition, as expected, also titers to other GII.4's increased due to cross-reactivity. Further, also very high IgG titers to GII.12 were detected at month 12. This suggests that the child was also infected with norovirus GII.12 between the 2 sampling dates. No infection of GII.12 was identified, however, infections were only evaluated if the child had symptomatic disease. The results would indicate that the child was indeed infected with GII.12, however, that the infection was asymptomatic. Additionally, increased IgG titers were detected for several other GII VLPs (such as, for instance, GII.17), although overall lower than titers detected against GII.12 and GII.4 Sydney. These increased IgG titers are believed to be the result from cross-reactive antibodies, although additional exposures to those noroviruses might be possible. Enhanced cross-reactivity against multiple VLPs is believed to be promoted by exposures to multiple different norovirus types. Thus, adults are in general much less susceptible to norovirus disease than children, as adults have been exposed to an increased number of different noroviruses, also increasing the number of cross-reactive antibodies.

TABLE 7
IgG, IgM, and IgA antibody titers (EC₅₀) in samples from subject #D against different norovirus VLPs
as measured by the 20-plex non-competitive assay set-up. Samples were taken at 5 and 12 months after birth.

| Timepoint | | | | | | | | | | | |
|---|---|---|---|---|---|---|---|---|---|---|---|
| (months) | Ig | GI.1 | GI.2 | GI.3 | GI.4 | GI.5 | GI.6 | GI.7 | GII.1 | GII.2 | GII.3 |
| 5 | IgA | 0 | 0 | 0 | 0 | 0 | 0 | 0 | 0 | 0 | 0 |
| 12 | IgA | 0 | 0 | 0 | 0 | 1 | 0 | 0 | 0 | 0 | 0 |
| 5 | IgG | 4 | 13 | 2 | 4 | 6 | 29 | 5 | 65 | 15 | 27 |
| 12 | IgG | 36 | 31 | 35 | 5 | 45 | 22 | 45 | 683 | 71 | 344 |
| 5 | IgM | 4 | 7 | 14 | 7 | 4 | 2 | 14 | 16 | 10 | 21 |
| 12 | IgM | 4 | 1 | 2 | 1 | 1 | 1 | 3 | 7 | 2 | 3 |

| Timepoint | | | | | GII.17/ | GII.17/ | GII.4/ | GII.4/Den | GII.4/ | GII.4/New | GII.4 |
|---|---|---|---|---|---|---|---|---|---|---|---|
| (months) | Ig | GII.6 | GII.7 | GII.12 | 1978 | 2015 | Consensus | Haag | Yerseke | Orleans | Sydney |
| 5 | IgA | 5 | 0 | 0 | 0 | 0 | 0 | 0 | 0 | 0 | 0 |
| 12 | IgA | 0 | 0 | 36 | 0 | 0 | 8 | 17 | 0 | 10 | 31 |
| 5 | IgG | 21 | 31 | 64 | 6 | 9 | 34 | 53 | 50 | 27 | 40 |
| 12 | IgG | 304 | 206 | 6657 | 209 | 432 | 875 | 2691 | 741 | 870 | 4741 |
| 5 | IgM | 31 | 17 | 16 | 10 | 48 | 9 | 7 | 7 | 6 | 6 |
| 12 | IgM | 36 | 4 | 9 | 2 | 24 | 9 | 12 | 6 | 5 | 13 |

The results confirm that the 20-plex assay set-up can identify norovirus infections that have caused disease, but also indicates that asymptomatic infections can be detected. Information on the antibody response in children to symptomatic and asymptomatic infections, as well as the evolution of the cross-reactive responses are valuable to identify patterns that may indicate protection from disease, and additionally assess responses to vaccination.

### Example 4: Determination of Antibody Titers in a Competitive Microsphere Immunoassay Set-Up

In order to determine the amount of specific norovirus-reactive antibodies in human samples, a competitive single- or multiplex microsphere immunoassay set-up was developed. In brief, human serum samples were incubated with one or more different microspheres coupled to different norovirus VLPs to allow binding of the norovirus-reactive Abs in the sample to the corresponding one or more norovirus VLPs. Afterwards, one or more different anti-norovirus reporter antibodies were added in order to compete with the specific norovirus-reactive antibodies in the sample for VLP binding. Then, a secondary reporter antibody coupled to phycoerythrin (PE) directed against the reporter antibody was added to detect amounts of reporter antibody bound to the VLPs. Thereby, the presence and/or amount of specific norovirus-reactive antibodies in the sample competing with the reporter antibody for VLP binding can be determined. Alternatively, direct labeling of the reporter antibody with PE is also possible, thereby avoiding the need for applying a secondary reporter antibody. “Specific” within that context means that the detected norovirus-reactive antibodies in the sample are capable of competing with the reporter antibody for VLP binding.

This assay set-up allows evaluation and characterization of a specific acute and convalescent immune response after single or multiple norovirus infections or after vaccination against norovirus. By determining the specific immune status, natural infection and vaccination can be distinguished. In addition, progression of the specific immune response and changes of the specific immune status over time can be analyzed. The assay is further suited to determine whether titers of specific antibodies are protective or not by comparing to titers of specific antibodies from protected individuals. Moreover, the assay enables monitoring cross-reactive antibody responses over time after infection with a certain norovirus type or vaccination by application of a cross-reactive reporter antibody. In addition, the assay enables to evaluate changes in patterns of specific antibodies after a second or further norovirus infection. If a norovirus-neutralizing and/or norovirus-blocking reporter antibody is applied, also norovirus-neutralizing and/or norovirus-blocking antibodies in the sample can be detected, as antibodies competing with the norovirus-neutralizing and/or norovirus-blocking reporter antibody will most likely also be neutralizing and/or blocking.

### Example 4.1: Production of Anti-GII.4/Sydney Reporter mAbs in Mice and Characterization of the Same

In a first step, reporter antibodies directed against GII.4/Sydney norovirus (cf. Table 1) were produced.

### Immunization and Hybridoma Production

Therefore, four female CD2F1 mice were immunized with GII.4/Sydney VLP (5 μg per dose) using an oil-in-water emulsion as adjuvant (Sigma Adjuvant System, Sigma Aldrich, Cat. No. S6322-1VL) and histidine buffer as vehicle. This adjuvant was designed for use in mice and is derived from bacterial and mycobacterial cell wall components that provide potent stimulus to the immune system. Each adjuvant vial contains 0.5 mg Monophosphoryl Lipid A (detoxified endotoxin) from Salmonella minnesota and 0.5 mg synthetic Trehalose Dicorynomycolate in 2% oil (squalene)-Tween-80-water. The immunogen was injected into the hock, the lateral tarsal region just above the ankle, a non-weight bearing structure draining to the same lymph node as the footpad (Kamala, J. Immunol. Methods 2007, 328 (1-2): 204-214) on days 0, 3, 7, 10, 14, 18, 21, and 28.

On day 28, spleens and popliteal lymph nodes were harvested and B-cells were isolated by tissue grinding and washing. B-cells were fused with P3U1 myeloma cells using electroporation. Cells were passaged in HAT (hypoxanthine-aminopterin-thymidine) medium for selection of fused cells. 1036 colonies were selected and each colony was passaged further.

### Primary Antigen Binding Screen Using ELISA

Hybridoma supernatants were screened in an Enzyme Linked Immunosorbent Assay (ELISA) using GII.4/Sydney VLP-coated plates. Undiluted supernatants were added to the plates and incubated for 1 hour at room temperature. After incubation, mAb binding was detected using a goat anti-mouse Ab coupled to horseradish peroxidase (HRP) and 2,2′-azino-bis(3-ethylbenzothiazoline-6-sulfonic acid (ABTS) as substrate. Fifty-five hybridoma clone supernatants which resulted in OD values greater than 0.4, which was considered to be a clear positive signal, were selected for propagation. As positive control, sera from terminal mice bleeds were included. In a second screen, the supernatants of the 55 hybridoma cells were tested in an ELISA as described above, using GII.4/Sydney and in addition GII.4/Consensus VLPs coated to the plates. 40 hybridoma clones showing binding to GII.4/Sydney and GII.4/Consensus VLPs were chosen for further characterization.

Primary Evaluation of mAbs in PGM Blockade Assay

Selected hybridoma supernatants were further screened in a Pig Gastric Mucin (PGM) blockade assay as described previously (Haynes et al., Viruses 2019, 11, 392, doi:10.3390/v11050392). In brief, Maxisorp plates (Thermo Fisher Scientific, Waltham, MA, USA) were coated with 100 μL of a 5 μg/mL solution of PGM (Sigma-Aldrich, Natick, MA, USA) in PBS (Thermo Fisher Scientific, Waltham, MA, USA) overnight at 4° C., or 1 h at 37° C. Following coating, plates were washed 3 times with 300 μL/well PBS and 0.05% Tween 20 (PBST), and then blocked with 200 μL/well of StartingBlock (PBS) Blocking Buffer (Thermo Fisher Scientific, Waltham, MA, USA) for 1 h at room temperature. Plates were washed 3 times with PBST before use. Undiluted hybridoma supernatants were mixed with GII.4/Sydney, GII.4/Consensus, GII.4/Yerseke, GII.17/2015, and GII.1 VLPs (cf. Table 1), respectively, and incubated overnight at 22° C. The mixture was transferred to the PGM-coated plates including VLP-only controls and incubation carried out for 1 h at 22° C. or 37° C. Following 3 washes with PBST, detection antibody specific for the corresponding VLP was added and the plates were incubated at room temperature for 1 h and then washed 3 times with PBST. A goat anti-rabbit IgG-HRP (Southern Biotech, Birmingham, AL, USA; #4030-05) secondary antibody was then added and incubation carried out for 1 h at room temperature. Following 3 washes, enzyme substrate (ABTS, KPL) was added and allowed to react for 12 min at room temperature. ABTS Peroxidase Stop Solution (KPL) was then added and plates were read at a wavelength of 450 nm in a Molecular Devices plate reader using SoftMax Pro Software (Molecular Devices, Downingtown, PA, USA) to obtain the Optical Density (GD) of each well. Percentage of VLP blocking was calculated with reference to GD values from VLP-only controls (Table 8). None of the mAbs blocked the binding of GI.I/Norwalk VLP suggesting that these Abs are not cross-reactive across genogroups. Strong binding against GII.4/Sydney VLP was observed. Moreover, all mAbs blocked GII.4/Consensus VLP binding.

TABLE 8
Blocking of VLP binding by mice mAbs evaluated in a PGM blockade
assay. Hybridoma supernatants from 40 clones were analyzed
for binding to different VLPs in the PGM blockade assay. Percentage
of blocking of VLPs in PGM binding was calculated with reference
to a VLP-only control without antibody.

% Blocking

| | GII.4/ | GII.4/ | GII.4/ | GII.17/ | |
|---|---|---|---|---|---|
| Clone | Sydney | Consensus | Yerseke | 2015 | GI.1 |
| 01A01 | 81.4 | 74.8 | 10.5 | 7 | 0 |
| 01E02 | 72 | 55.9 | 8.9 | 0 | 0 |
| 02A02 | 79.7 | 67.9 | 6.6 | 7.2 | 0 |
| 02A04 | 85.9 | 78.2 | 12.4 | 33.7 | 0 |
| 02A05 | 81.8 | 74.9 | 11.5 | 0 | 0 |
| 02C12 | 35 | 35.8 | 5.5 | 0 | 0 |
| 02D05 | 93 | 67.7 | 18.1 | 0 | 0 |
| 02E07 | 93.1 | 46.4 | 6.3 | 3.6 | 0 |
| 03B01 | 92.6 | 70 | 12 | 0 | 0 |
| 03C02 | 60 | 48 | 2.3 | 2.7 | 0 |
| 03F09 | 92.6 | 81.2 | 14.3 | 27.3 | 0 |
| 04H04 | 86.6 | 78.5 | 5.8 | 1.5 | 0 |
| 04E06 | 75.3 | 58.7 | 25.6 | 13.6 | 0 |
| 04H06 | 93 | 45.6 | 6 | 0 | 0 |
| 05A01 | 81.5 | 78.1 | 5.6 | 21.5 | 0 |
| 05A03 | 80.3 | 74.5 | 9 | 3.1 | 0 |
| 05A04 | 92.8 | 44.9 | 9.4 | 12 | 0 |
| 05A05 | 73.2 | 57.8 | 7.1 | 5.9 | 0 |
| 05B01 | 52.5 | 43.8 | 5.9 | 11.5 | 0 |
| 05C06 | 64 | 48.8 | 4.7 | 0 | 0 |
| 05B08 | 85.8 | 78.5 | 11.7 | 32.1 | 0 |
| 05D08 | 93 | 43.7 | 10 | 0.5 | 0 |
| 05E09 | 92.6 | 80.1 | 8.7 | 0 | 0 |
| 05E10 | 78.9 | 57.5 | 16.5 | 18 | 0 |
| 05G05 | 78.4 | 66.3 | 9.7 | 0 | 0 |
| 06D04 | 78.8 | 62.1 | 7.6 | 4.4 | 0 |
| 06F05 | 92.2 | 88.9 | 0 | 0 | 0 |
| 07C04 | 79.1 | 69.9 | 7.5 | 0 | 0 |
| 07H04 | 87.2 | 77.7 | 8.6 | 0 | 0 |
| 08B04 | 78.8 | 59.3 | 9.5 | 18.8 | 0 |
| 08D05 | 92.8 | 42.4 | 6.3 | 0 | 0 |
| 08A07 | 77.5 | 71.2 | 3.7 | 4.6 | 0 |
| 08A08 | 86.1 | 77.9 | 14.4 | 43.7 | 0 |
| 08C09 | 92.3 | 88.8 | 86 | 0 | 0 |
| 08B12 | 93 | 45.3 | 4.6 | 5.1 | 0 |
| 09C03 | 93 | 89 | 88.8 | 0 | 0 |
| 09E10 | 53.4 | 46 | 8.4 | 0 | 0 |
| 10B01 | 91.5 | 78.5 | 6.1 | 26.4 | 0 |
| 11F03 | 92.9 | 69.1 | 18.8 | 0 | 0 |
| 11H04 | 92.9 | 58.9 | 0.1 | 0 | 0 |

### Antibody Purification

For antibody purification from hybridoma supernatants, a 5000 slurry of Protein A Sepharose (GE 17127903) in Phosphate Buffered Saline (PBS; Gibco 14190-144) was prepared. 600 μL of the slurry were subsequently added to 50 mL of hybridoma supernatant in case of isotypes IgG2a, 2b, and 3. Alternatively, 300 μL of the slurry mixed with 25 mL Protein A MAPSII binding buffer (BioRad 153-6161) were added to 25 mL of hybridoma supernatant in case of IgG1. Samples were incubated overnight at 4° C. and centrifuged at 1800 rpm for 5 min the next day. Supernatant was removed and beads were washed using an Unifilter (24 Well 10 mL GE 7700-9904 15057065) and either MAPSII binding buffer (IgG1) or PBS (other isotypes). Elution carried out with 0.1 M glycine, 0.3 M NaCl pH 3.0. Finally, buffer was exchanged into PBS using an Amicon Ultra 15 MWCO 30 kDa concentrator. Antibody concentration was determined by measuring absorbance at 280 nm.

### Epitope Binning

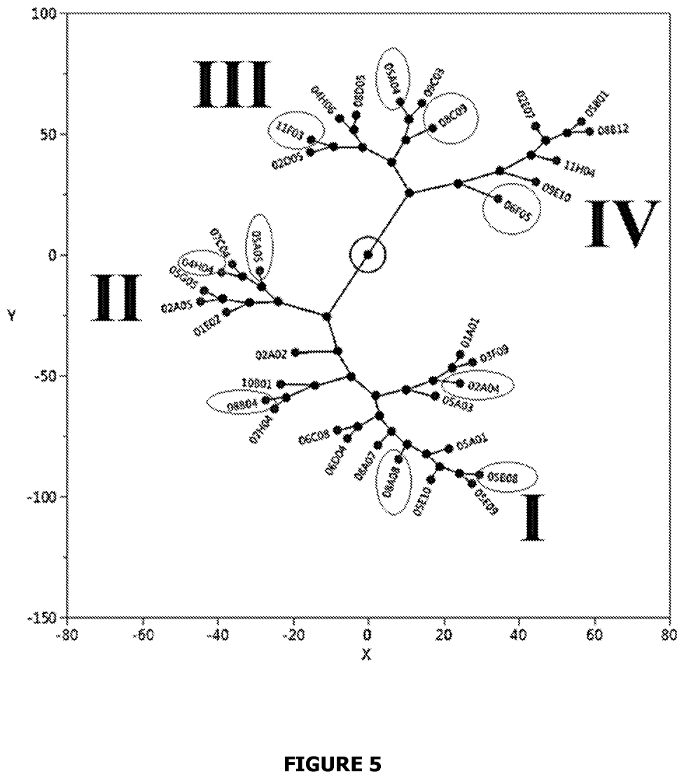

Epitope binning of 35 out of the 40 mAbs was performed with Bio-Layer Interferometry (BLI) using an Octet system following standard procedures. For BLI, purified antibodies were applied (see section above for purification). BLI technology allows to characterize and sort Abs into bins that indicate binding of distinct epitopes on a specific antigen. Briefly, the first mAb (stock concentration of 20 μg/mL) was immobilized on an anti-mouse IgG Fc Capture (AMC) biosensor. Blocking was performed using a mouse polyclonal IgG control (stock concentration of 50 μg/mL). GII.4/Sydney VLPs (stock concentration of 1 μg/mL) were incubated with the second mAb (stock concentration of 40 μg/mL) at a 1:16 (w/w) ratio of VLP to mAb. Afterwards, this mixture was added to the biosensors and responses were measured. In addition, a control was included using solely VLP without incubation with the second mAb. Binding values for each mAb were calculated by dividing the binding response by incubation with VLP pre-incubated with the second mAb by the binding response by incubation with VLP only. Analysis revealed clustering of the mAbs into 4 bins (FIG. 5) and 10 mAbs (02A04, 04H04, 05A04, 05A05, 05B08, 06F05, 08A08, 08B04, 11F03, 08C09) were selected for further evaluation.

Evaluation of Selected mAbs in PGM Blockade Assay

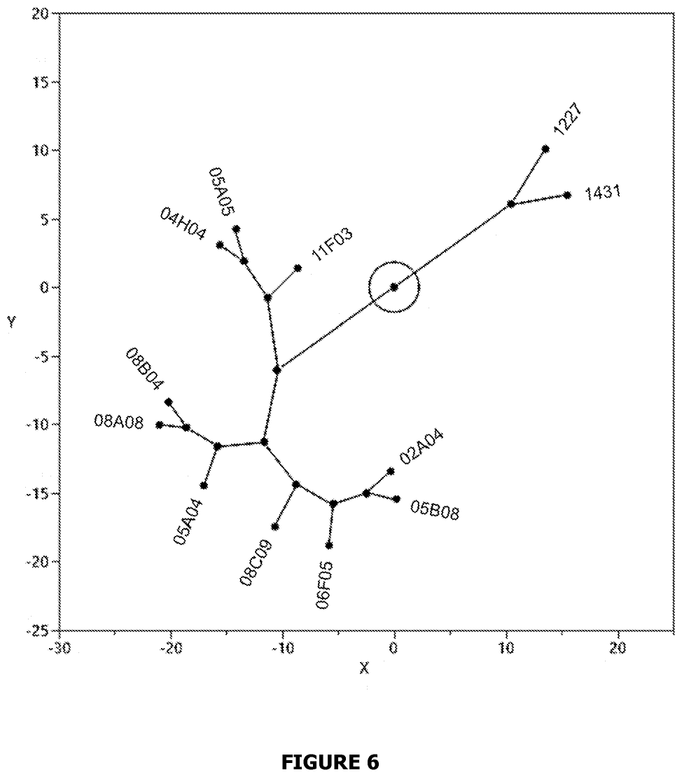

The 10 purified mAbs selected from epitope binning were again screened in the PGM blockade assay (Haynes et al., Viruses 2019, 11, 392, doi:10.3390/v11050392) using a larger panel of norovirus VLPs. In addition, two human mAbs (for sequence information, reference is made to the Annex section; regarding the two mAbs reference is made to Lindesmith et al, Immunity 2019, 50:1530-1541 (A1227 and A1431)) were included. In addition, epitope binning was performed as described above using the 10 selected mAbs and the human mAbs (FIG. 6).

The PGM blockade assay was performed as described above, except that the mAbs were serially diluted and incubated overnight with each of GII.6, GII.17/2015, GII.4/Sydney, GII.4/Yerseke, GII.4/Consensus, GII.4/Den Haag, GII.4/Houston, and GII.4/New Orleans VLPs at 22° C. The following, day corresponding dilutions incubated with VLPs were transferred to the PGM-coated plates. The OD data was curve fit and blocking titers were calculated as the supernatant dilution interpolated at 1/2 the maximum OD for the plate. The blocking titers represent the supernatant dilution that produces a 50% reduction in VLP binding to PGM. The maximum OD for the plate was calculated from the VLP only control. Blocking titers (Table 9) show that clones 06F05, 11F03, and 05A04 strongly block binding of GII.4/Sydney VLPs. Clone 06F05 additionally showed a similar blocking titer towards GII.4/Consensus VLPs. A high blocking titer was also observed for the human mAb 1431 for GII.4/Consensus VLPs, as may be expected because the human monoclonal antibodies were selected by their ability to recognize the GII.4/Consensus VLP.

TABLE 9
Blocking titers of mAbs against different GII.4 VLPs evaluated by a PGM assay. Missing values
indicate that sigmoidal fitting was not possible due to insufficient antibody binding.

| | mAb | | | | | | | | | | | |
|---|---|---|---|---|---|---|---|---|---|---|---|---|
| VLP | 02A04 | 04H04 | 05A04 | 05A05 | 05B08 | 06F05 | 08A08 | 11F03 | 08B04 | 08C09 | 1431 | 1227 |
| Sydney | 2943 | 697 | 337156 | 91 | 954 | 115497 | 3536 | 107714 | 5271 | 6080 | 7277 | |
| Consensus | | | | | | 156091 | | 2174 | 4476 | 4798 | 139073 | 447 |
| Den Haag | | | | | | 1166 | | | | | 5718 | 162 |
| Yerseke | | | | | | | | | | | 4853 | |
| New Orleans | | | | | | | | | | | | |
| Houston | | | | | | | | | | 15 | 12114 | |

Evaluation of Selected mAbs in a Microsphere Immunoassay Set-Up

The 10 mAbs selected from epitope binning experiments (02A04, 04H04, 05A04, 05A05, 05B08, 06F05, 08A08, 08B04, 11F03, 08C09) and the two human mAbs 1431 and 1227 were further evaluated for binding to different norovirus VLPs in a multiplex microsphere immunoassay set-up using different norovirus VLPs.

VLP-coupled microspheres were prepared as described under Example 2. A working microsphere mixture was prepared by diluting the coupled microsphere stock to a final concentration of 30 microspheres/μL in assay buffer (1% (w/v) BSA in 1-fold PBS, pH 7.4). The working mixture was kept at room temperature until further use. Monoclonal antibodies were diluted in assay buffer to 37.5 μg/mL. 125 μL of diluted monoclonal antibody were added per well to the first column of a black flat bottom 96 well plate (Corning Inc.) and 100 μL of assay buffer were added per well to the rest of the wells. The monoclonal antibodies were serially diluted across the plate by transferring 25 μL from column 1 to 2 and so on. 50 μL/well of the microsphere working mixture were added to all wells of the plate. The plate was covered with a foil sealing sheet and incubated for 60 min (±5 min) at room temperature on a plate shaker at 600 rpm. After incubation, the assay plate was washed two times with PBS-T (0.05% (v/v) Tween-20 in PBS pH 7.4) in a magnetic plate washer (BioTek Instruments, Product Id. 400072). The plate was placed in a 96-well plate magnet (Life Technologies, Product Id. 32513) and incubated for 30 sec while covering the plate. Following the incubation step, the supernatant was removed. For detection, R-PE AffiniPure F(ab′)₂ fragment goat anti-mouse IgG detection Ab (heavy and light chain; Jackson ImmunoResearch, Cat. No. 115-116-146, Lot. No. 143867, 0.5 mg/mL) was diluted 1:100 in assay buffer to achieve a final working concentration of 5 μg/mL by vortexing for 5 sec. 100 μL of the diluted detection Ab were added to each well. The plate was covered with a foil sealing sheet and incubation carried out for 1 hour (±2 min) at room temperature on a plate shaker at 600 rpm. The assay plate was washed two times with PBS-T in the magnetic plate washer. After the washing steps, the plate was placed in the 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. is possible. Prior to sample read-out, the plate is allowed to re-equilibrate to room temperature for 20 min (±5 min) if stored overnight at 4° C. The plate was put on an orbital shaker at 600 rpm for at least 5 min in order to allow for complete resuspension of microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex. The microsphere count was set to 50, i.e. the instrument analyzed at least 50 microspheres per microsphere type (e.g. at least 50 microspheres coupled to GII.4/Consensus).

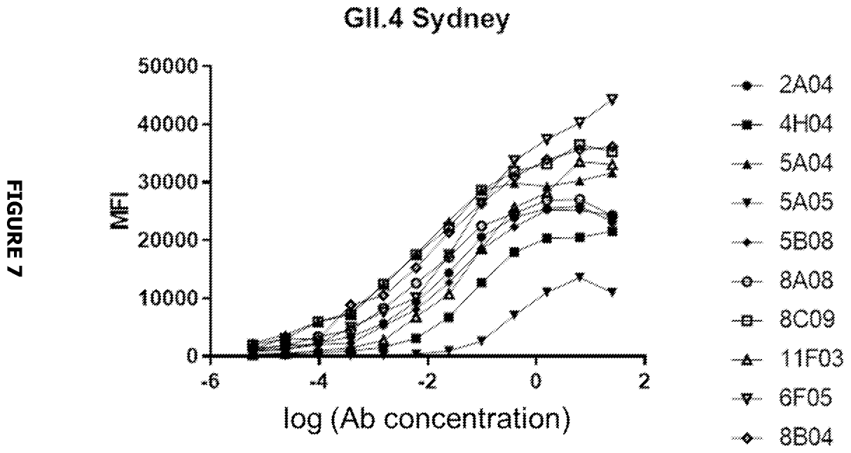

Data were analyzed and plotted using GraphPad Prism 8 version 8.1.0 (GraphPad Software, Inc). Binding curves of the mAbs are exemplarily shown for GII.4/Sydney VLPs (FIG. 7). Sigmoidal fitting according to a dose-response curve (Sigmoidal, 4PL, X=Log(concentration)) carried out by Log-transformation and interpolation analysis of Median Fluorescent Intensity (MFI). The equation used for the non-linear regression was “log(agonist) vs. response-Variable slope”. EC₅₀ (effective concentration at which 50% of mAb binds to the antigen-coupled microspheres) values were calculated for each mAb in combination with all investigated norovirus VLP-coupled microspheres (Table 10).

TABLE 10
Evaluation of mAb binding towards norovirus VLP-coupled microspheres. Presented are the
EC₅₀ values (μg/mL) for all mAb towards the different VLPs. Missing values indicate
that sigmoidal fitting was not possible due to insufficient binding of the mAb to the VLP.

| | mAbs | | | | | | | | | | | |
|---|---|---|---|---|---|---|---|---|---|---|---|---|
| VLP | 2A04 | 4H04 | 5A04 | 5A05 | 5B08 | 8A08 | 8C09 | 11F03 | 6F05 | 8B04 | 1431 | 1227 |
| GII.1 | | 1.632 | | | | 0.376 | 0.311 | | | 0.276 | | 0.021 |
| GII.2 | 0.632 | 0.264 | | 0.450 | 0.404 | 0.395 | | | | | | 0.105 |
| GII.3 | 0.243 | | | | 0.097 | 0.507 | 0.038 | | | 0.047 | | 0.006 |
| GII.4/Consensus | 0.022 | 0.139 | | 0.122 | 0.035 | 0.054 | 0.045 | | 0.062 | 0.041 | 0.070 | 0.216 |
| GII.4/Den Haag | 0.015 | | | | 0.021 | 0.009 | 0.010 | 1.834 | 0.040 | 0.016 | 0.024 | 0.014 |
| GII.4/New Orleans | 0.013 | 0.089 | | 0.315 | 0.019 | 0.008 | 0.006 | | 0.070 | 0.010 | 0.018 | 0.007 |
| GII.4/Sydney | 0.016 | 0.065 | 0.004 | 0.302 | 0.025 | 0.009 | 0.009 | 0.082 | 0.041 | 0.014 | 0.026 | 0.012 |
| GII.4/Yerseke | 0.010 | 0.212 | | 0.460 | 0.015 | 0.008 | 0.002 | | | 0.004 | 0.007 | 0.002 |
| GII.6 | 0.144 | | | | 0.404 | 0.095 | 0.031 | | | 0.042 | | 0.071 |
| GII.7 | 0.043 | 1.008 | | | 0.085 | 0.017 | | | | | | 0.009 |
| GII.12 | | | | 0.881 | 1.613 | 0.247 | | | | | | 0.026 |
| GII.17/1978 | | 1.613 | | | | | | | | | | 0.013 |
| GII.17/2015 | | | | | | | | | | | | 0.012 |

Sigmoidal fitting was not possible due to insufficient or absent binding of the mAbs to any norovirus VLPs of genogroup I (GI.1, GI.2, GI.3, GI.4, GI.5, GI.6, and GI.7) with the exception of human mAb 1227, which provided EC₅₀ values of 0.003, 0.002, 0.002, 0.002, 0.001, 0.002, and 0.021 μg/mL towards GI.1 VLP, GI.2 VLP, GI.3 VLP, GI.4 VLP, GI.5 VLP, GI.6 VLP, and GI.7 VLP, respectively. All mAbs showed strong binding to GII.4/Sydney/2012 VLPs.

### Example 4.2: Evaluation of Human Serum Samples in a Singleplex Competitive Microsphere Immunoassay Set-Up Using the Anti-GII.4/Sydney Reporter mAbs

The 10 mAbs selected from epitope binning (02A04, 04H04, 05A04, 05A05, 05B08, 06F05, 08A08, 08B04, 11F03, 08C09) (cf. Example 4.1) were further used for evaluation of different commercial human sera (Bioreclamation IVT Catalog #HUMANSRM1800041, Westbury, NY) in a singleplex competitive microsphere immunoassay set-up using GII.4/Sydney VLP-coupled microspheres. Singleplex within that context means that per well one VLP and one reporter mAb are applied. The serum donors were non-vaccinated donors, who may have been exposed to norovirus.

Serum samples were pre-characterized with the PGM blockade assay as described under Example 4.1 “Evaluation of selected mAbs in PGM blockade assay”, using GII.4/Sydney VLPs and serially dilutions of sera in order to determine blocking titers (Table 11). Based on blockade activity, No. 797 was assigned as a negative control.

GII.4/Sydney VLP-coupled microspheres were prepared as described under Examples 1 and 2. A microsphere working mixture was prepared by diluting the coupled microsphere stock to a final concentration of 30 microspheres/μL in assay buffer (1% (w/v) BSA in 1-fold PBS, pH 7.4). The working mixture was kept on ice until further use. 30 μL of undiluted stock sera were added per well to column 1 of a black flat bottom 96 well assay plate (Corning Inc.). 120 μL of assay buffer were added per well to all wells in column 1 and 100 μL per well to the rest of the plate. The sera were diluted 1:3 down the plate by taking 50 μL from row A and adding to row B, and so on. The last 50 μL were discarded. A monoclonal-only control without serum was included by pipetting 100 μL assay buffer into the last column of the plate to measure the upper limit of mean fluorescence intensity (MFI) and the maximum binding of mAb to the VLP. Then, 50 μL of the microsphere working mixture were added to all wells of the plate. The plate was covered with a foil-sealing sheet and incubated overnight at 4° C. After incubation with serum samples, the microspheres were washed two times with PBS-T in a magnetic plate washer.

mAbs were diluted to 0.05 μg/mL in assay buffer. 100 μL of the diluted mAbs were added per well to all the wells. No additional mixing was performed. The plate was covered with a foil sealing sheet and incubated for 1 hour±5 min at room temperature on a plate shaker at 600 rpm. After incubation, the assay plate was washed two times with PBS-T in a magnetic plate washer. The plate was placed in a 96-well plate magnet and incubated for 30 sec while covering the plate. Following the incubation step, the supernatant was removed. For detection of mouse mAbs, a R-PE AffiniPure F(ab′)₂ fragment goat anti-mouse IgG (heavy and light chain; Jackson ImmunoResearch, Cat. No. 115-116-146, Lot. No. 143867, 0.5 mg/mL) detection Ab was applied. The detection Ab was diluted 1:100 in assay buffer to achieve a final working concentration of 5 μg/mL by vortexing for 5 sec. 100 μL of the diluted detection Ab were added to each well. The plate was covered with a foil sealing sheet and incubation carried out for 1 hour (±5 min) at room temperature on a plate shaker at 600 rpm. The assay plate was washed two times with PBS-T in a magnetic plate washer. After the washing steps, the plate was placed in a 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. was possible. Before sample read-out, the plate was allowed to re-equilibrate to room temperature for 20 min (±5 min) if stored at 4° C. overnight. The plate was put on an orbital shaker at 600 rpm for at least 5 min in order to allow for complete resuspension of microspheres. The plate was placed in the multiplexing MAGPIX® plate reader (Luminex Corporation, Austin, Texas). The xPONENT® (Build 4.2.1705.0) program was used with sample volume set to 50 μL per well; plate protocol: 96-well plate format; and microsphere protocol: Map, BP 50 regions, Type MagPlex. The microsphere count was set to 50, i.e. the instrument analyzed at least 50 microspheres per microsphere type.

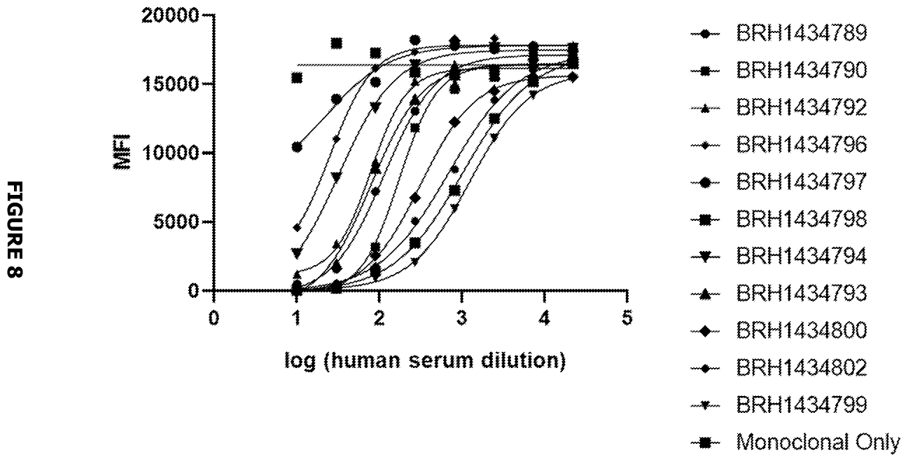

Data were analyzed and plotted using GraphPad Prism 8 version 8.1.0 (GraphPad Software, Inc.). First, an average of all values of the monoclonal-only control was calculated. A non-linear regression fit [Sigmoidal, 4PL, X=Log(plasma dilution)] of MFI values for all sera was performed and data was interpolated at 50% value of monoclonal-only control. These values were reported as interpolation titers in Table 11. Competition curves are exemplarily shown for mAb 11F03 (FIG. 8). Interpolation titers are indicative for the ability of each serum to block the binding of the mAbs to the norovirus VLPs coupled to the microspheres. Interpolation was not possible for the negative control serum No. 797, indicating a high degree of mAb binding, independent of serum dilution, as no anti-norovirus Abs were present within this sample. In contrast, higher titers were expected for human serum samples from donors which were pre-exposed to norovirus, as norovirus specific Abs were expected to be present in these samples, validating the assay set-up. Several samples showed blocking towards all or most of the mAbs (e.g. No. 799, 798, and 802), indicating that multiple types of selective and/or cross-reactive antibodies are present within the sample.

TABLE 11
Blocking titers and interpolation titers for human sera evaluated in a PGM blockade assay using GII.4/Sydney/2012
VLPs and in a competitive microsphere immunoassay using different mAbs and GII.4/Sydney/2012 VLPs, respectively.
Missing values for the microsphere immunoassay indicate that no interpolation was possible due to insufficient
competition. Serum sample No. 797 was assigned as a negative control.

| | | | | | | | | | | | Blocking titers |
|---|---|---|---|---|---|---|---|---|---|---|---|
| Serum Samples | Interpolation titers | | | | | | | | | | (PGM blockade |
| (BRH1434-No.) | 8C09 | 5A04 | 5A05 | 2A04 | 6F05 | 08A08 | 05B08 | 04H04 | 08B04 | 11F03 | assay) |
| No. 797 | | | | | | | | | | | 0 |
| No. 795 | | | 23 | 17 | | 10 | 16 | | 18 | | 0 |
| No. 805 | | | 29 | 21 | | 16 | 20 | | 22 | | 0 |
| No. 791 | | | 28 | 22 | | 16 | 20 | 17 | 28 | | 0 |
| No. 803 | | | 22 | 40 | | 21 | 53 | 6 | 44 | | 0 |
| No. 808 | | | 72 | 82 | | 48 | 89 | | | | 0 |
| No. 801 | 10 | | 87 | 103 | | 41 | 64 | | 62 | 15 | 0 |
| No. 796 | | 9 | 89 | 47 | | 41 | 79 | 65 | 87 | 46 | 44 |
| No. 806 | | | 65 | 32 | | 22 | 78 | 18 | 61 | 68 | 45 |
| No. 804 | 29 | | 86 | 166 | 28 | 58 | 186 | 13 | 150 | 95 | 145 |
| No. 794 | 40 | | 147 | 134 | | 117 | 175 | 139 | 209 | 63 | 159 |
| No. 792 | 22 | | 148 | 87 | 22 | 94 | 190 | 174 | 173 | 117 | 185 |
| No. 807 | 29 | | 349 | 336 | 94 | 247 | 781 | 79 | 378 | 146 | 225 |
| No. 793 | 22 | | 493 | 267 | | 230 | 385 | 342 | 436 | 201 | 229 |
| No. 789 | 53 | 16 | 417 | 256 | 79 | 222 | 359 | 337 | 385 | 229 | 358 |
| No. 790 | 119 | 26 | 280 | 428 | 323 | 289 | 315 | 348 | 470 | 271 | 397 |
| No. 800 | 123 | 29 | 227 | 484 | 258 | 200 | 382 | 405 | 505 | 399 | 507 |
| No. 799 | 406 | 213 | 2087 | 1850 | 831 | 1277 | 2172 | 1805 | 1474 | 755 | 1418 |
| No. 798 | 112 | 64 | 540 | 644 | 1124 | 400 | 774 | 917 | 647 | 972 | 1562 |
| No. 802 | 108 | 117 | 1388 | 2244 | 154 | 552 | 2116 | | 1592 | 853 | 2524 |

The blockade titers determined in the PGM blockade assay correlated well with the interpolation titers determined in the competitive microsphere immunoassay (Table 11), indicating that the competitive microsphere immunoassay provides a similar data outcome as the PGM blockade assay. In contrast to the PGM blockade assay, the competitive microsphere immunoassay set-up is for instance not limited to specific VLPs and enables parallel screening of multiple VLPs thereby facilitating and accelerating sample analysis.

### Example 4.3: Evaluation of Human Serum Samples in a Singleplex Competitive Microsphere Immunoassay Set-Up Using the Anti-GI.1 and Anti-GII.4/Consensus Reporter mAbs

In a next step, human serum samples from Example 4.2 were evaluated for Abs binding to GI.1 and GII.4/Consensus VLPs using a singleplex competitive microsphere immunoassay (Example 4.2) and anti-GI.1 or anti-GII.4/Consensus mAbs designated as 17-1-1 or 4-1-3, respectively. The anti-GI.1 and anti-GII.4/Consensus reporter mAbs were produced and tested using standard methods (see also Example 4.1). “Singleplex” within that context means that per well one VLP and one mAb are applied. Interpolation titers are shown in Table 12. Two sera were demonstrated to contain high titers of anti-GI.1 Abs (No. 805 and 806) and four sera to contain high titers of anti-GII.4 Consensus Abs (No. 790, 799, 798, and 802) respectively.

TABLE 12
Interpolation titers for human sera evaluated in a singleplex competitive microsphere
immunoassay using GI.1 and GII.4/Consensus VLPs and anti- GI.1 Norwalk (17-1-1) and
anti- GII.4 Consensus (4-1-3) mAbs, respectively. Missing values indicate that no
interpolation was possible due to insufficient competition (NC = negative control).

| Serum | mAbs and VLPs | | Serum | mAbs and VLPs | |
|---|---|---|---|---|---|
| Samples | 17-1-1 | 4-1-3 and | Samples | 17-1-1 | 4-1-3 and |
| (BRH1434-No.) | and GI.1 | GII.4/Consensus | (BRH1434-No.) | and GI.1 | GII.4/Consensus |
| No. 797 (NC) | | | No. 794 | 102 | |
| No. 795 | | 23 | No. 792 | 25 | 66 |
| No. 805 | 338 | | No. 807 | | 110 |
| No. 791 | | | No. 793 | | 74 |
| No. 803 | | | No. 789 | | 114 |
| No. 808 | | | No. 790 | | 231 |
| No. 801 | | | No. 800 | 59 | 68 |
| No. 796 | 43 | 52 | No. 799 | | 337 |
| No. 806 | 738 | | No. 798 | | 231 |
| No. 804 | 60 | 116 | No. 802 | | 404 |

### Example 4.4: Evaluation of Human Serum Samples in a Duplex Competitive Microsphere Immunoassay Set-Up Using the Anti-GI.1 and Anti-GII.4 Consensus Reporter mAbs

In a next step, human serum samples No. 797 (negative control, NC), 805, 806, 790, and 799 were further evaluated in a duplex competitive microsphere immunoassay. Duplex within that context means, that a mixture of GI.1 Norwalk and GII.4/Consensus VLPs coupled to the microspheres and anti-GI.1 Norwalk (17-1-1) and anti-GII.4 Consensus (4-1-3) mAbs was applied in one well. Thereby, anti-GI.1 Norwalk and anti-GII.4 Consensus Abs within a sample can be determined simultaneously in one well.

The selected sera were first analyzed with the PGM blockade assay as described under Example 4.1 “Evaluation of selected mAbs in PGM blockade assay”, using GI.1 and GII.4/Consensus VLPs and serially dilutions of sera in order to determine blocking titers (Table 13). Serum samples No. 805 and No. 806 showed higher blocking titers towards GI.1 Norwalk VLPs, whereas blocking titers were lower for GII.4 Consensus VLPs. Moreover, serum samples No. 790 and 799 showed high degree of blocking towards GII.4 Consensus VLPs, whereas no blocking titers could be determined for GI.1 Norwalk VLPs.

TABLE 13
Blocking titers of human serum samples examined in a PGM
blockade assay using GI.1 Norwalk and GII.4 Consensus VLPs.
Missing values indicate that sigmoidal fitting was not possible
due to lack of or low amounts of anti-GI.1 or GII.4/Consensus
antibody titers within the serum samples. Serum sample No.
797 was assigned as a negative control.

| | Serum samples | | | | |
|---|---|---|---|---|---|
| VLPs | No. 797 | No. 799 | No. 805 | No. 806 | No. 790 |

| | | | | |
|---|---|---|---|---|
| GI.1 | | 135 | 326 | |
| Norwalk | | | | |
| GII.4 | 1116 | 89 | 242 | 855 |
| Consensus | | | | |

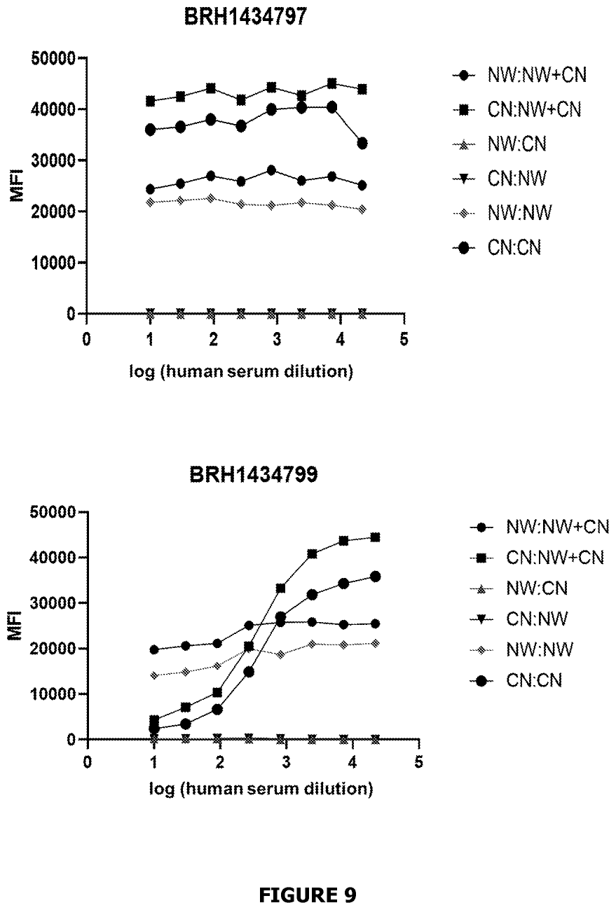

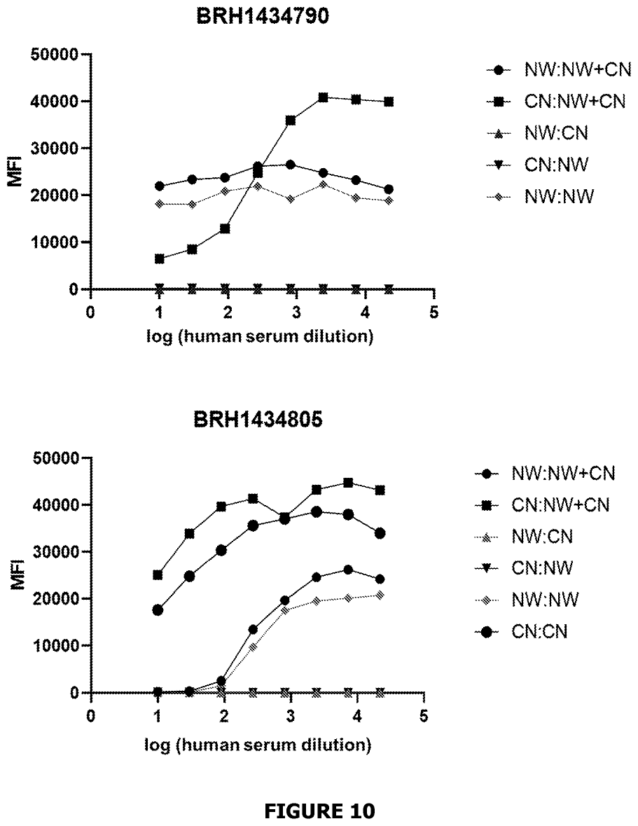

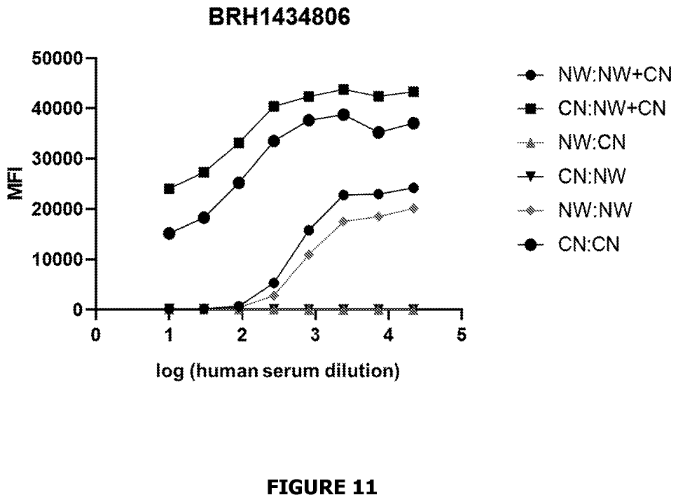

The duplex competitive microsphere immunoassay was carried out as described for the singleplex assay in Example 4.2, except for the fact that a mixture of GI.1 and GII.4/Consensus VLPs coupled to the microspheres and anti-GI.1 Norwalk (17-1-1) and anti-GII.4 Consensus (4-1-3) mAbs was applied per well. This means that 25 μL of each VLP working mixture (GI.1 and GII.4/consensus VLPs at 60 microspheres/μL each) were added per well, resulting in the same total VLP concentration per well as applied in the singleplex assay. Further, 50 μL of each diluted mAb (anti-GI.1 Norwalk and anti-GII.4 Consensus Abs at 0.1 μg/mL each) were applied per well, resulting in the same mAb concentration per well as applied in the singleplex assay. In addition, singleplex assay set-ups were included solely containing microspheres coated with either GI.1 or GII.4 Consensus/VLPs and either anti-GI.1 Norwalk (17-1-1) or anti-GII.4 Consensus (4-1-3) mAbs for comparison. Binding curves for evaluated serum samples are shown in FIGS. 9 to 11 and interpolation titers are shown in Table 14.

TABLE 14
Interpolation titers for human sera evaluated in singleplex and duplex
competitive microsphere immunoassay set-ups using GI.1 Norwalk and
GII.4 Consensus VLPs and anti-GI.1 Norwalk (17-1-1) and anti- GII.4
Consensus (4-1-3) mAbs. Missing values indicate that no interpolation was
possible due to insufficient competition. NW:NW + CN refers to the
titers resulting from analysis of GI.1 Norwalk VLP-coupled microspheres
present within a duplex set-up containing a mixture of GI.1 Norwalk and
GII.4 Consensus VLP-coupled microspheres and anti-GI.1 Norwalk
(17-1-1) and anti-GII.4 Consensus (4-1-3) mAbs. CN:NW + CN refers to
the titers resulting from analysis of GII.4 Consensus VLP-coupled
microspheres present within a duplex set-up containing a mixture of GI.1
Norwalk and GII.4 Consensus VLP-coupled microspheres and anti-
GI.1 Norwalk (17-1-1) and anti- GII.4 Consensus (4-1-3) mAbs. NW:CN,
NW:NW, CN:NW, CN:CN refer to singleplex set-ups with GI.1 Norwalk
VLP-coupled microspheres and anti-GII.4 Consensus (4-1-3) mAb, GI.1
Norwalk VLP-coupled microspheres and anti-GI.1 Norwalk (17-1-1) mAb,
GII.4 Consensus VLP-coupled microspheres and anti-GI.1 Norwalk
(17-1-1) mAb, and GII.4 Consensus VLP-coupled microspheres and
anti-GII.4 Consensus (4-1-3) mAb, respectively. For human sample
No. 790 and the CN:CN set-up, microspheres were lost during
assay workflow and therefore evaluation was not possible. Serum
sample No. 797 was assigned as a negative control.

Assay set-up

| (VLPs and | Serum sample | | | | |
|---|---|---|---|---|---|
| mAbs) | No. 797 | No. 799 | No. 805 | No. 806 | No. 790 |
| NW:NW + CN | | | 294 | 610 | |
| NW:CN | | | | | |
| NW:NW | | | 312 | 805 | |
| CN:NW + CN | | 294 | | | 209 |
| CN:NW | | | | | |
| CN:CN | | 381 | | 38 | Microspheres |
| | | | | | lost |

Singleplex and duplex data were comparable enabling multiplexing using different norovirus VLPs. Moreover, the singleplex data presented in Table 14 fitted the singleplex data in Table 12 underlining a low inter-assay variation.

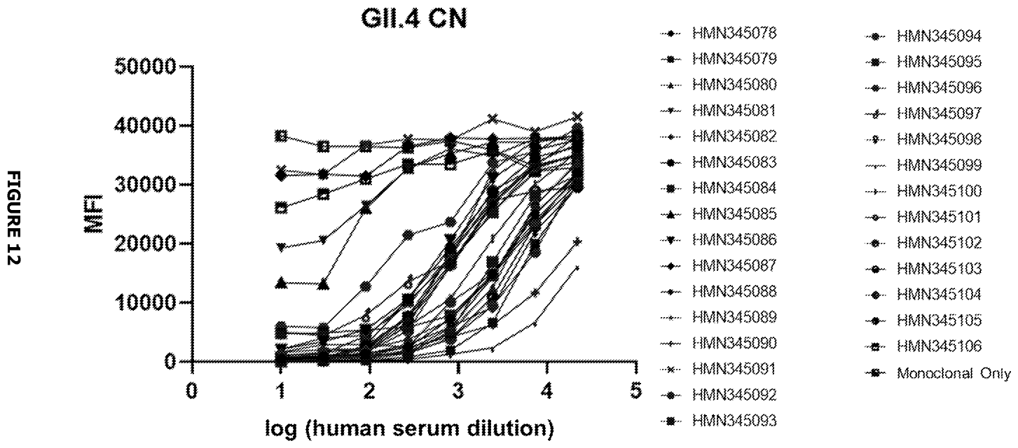

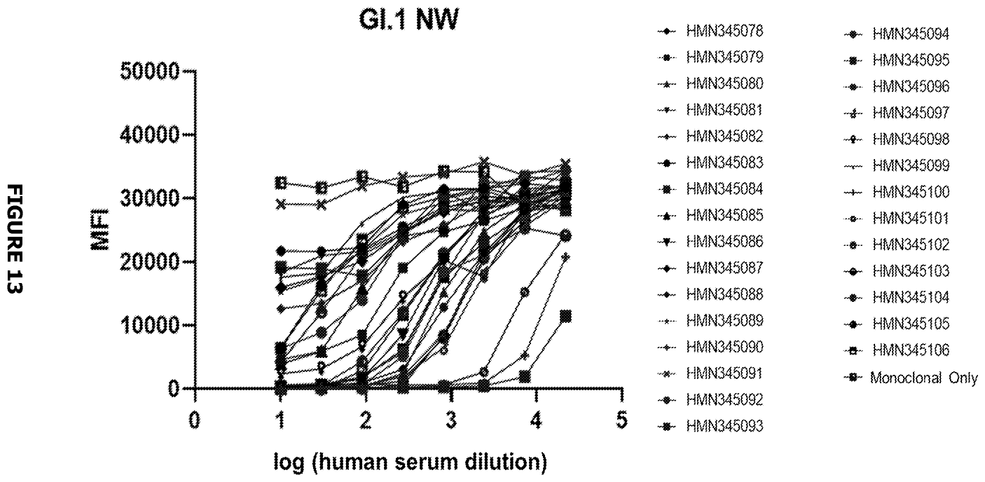

To expand evaluation of the duplex assay set-up further, another panel of other human serum samples (Bioreclamation IVT Catalog #S 10020004168, Westbury, NY) were evaluated for anti-GI.1 Norwalk and anti-GII.4 Consensus Abs (FIGS. 12 and 13). The results showed that the duplex assay is able to discriminate between samples that do not or only to a low amount contain anti-GI.1 Norwalk and anti-GII.4 Consensus Abs (i.e. “missing values”, e.g. sample EMN345081), samples that contain both antibody types at higher amounts (e.g. sample 34N345090), and samples that predominantly contain Abs directed against one of the two noroviruses (e.g. samples HMN345095 and HMN345099; Table 15).

TABLE 15
Interpolation titers for human sera evaluated in the
duplex competitive microsphere immunoassay set-up using
GI.1 Norwalk and GII.4 Consensus VLPs and anti- GI.1
Norwalk (17-1-1) and anti- GII.4 Consensus (4-1-3)
mAbs. Missing values indicate that no interpolation
was possible due to insufficient competition.

mAbs

| Serum | Anti-GII.4 | Anti-GI.1 |
|---|---|---|
| samples | Consensus | Norwalk |
| HMN345078 | 3405 | |
| HMN345079 | 6330 | 214 |
| HMN345080 | 3700 | 939 |
| HMN345081 | | |
| HMN345082 | 3388 | 70 |
| HMN345083 | 690 | 30 |
| HMN345084 | 2660 | |
| HMN345085 | 47 | 99 |
| HMN345086 | 617 | 548 |
| HMN345087 | | 8 |
| HMN345088 | 986 | 1214 |
| HMN345089 | 793 | 17 |
| HMN345090 | 16946 | 14639 |
| HMN345091 | | |
| HMN345092 | 3370 | 881 |
| HMN345093 | 918 | 852 |
| HMN345094 | 6849 | 110 |
| HMN345095 | 3987 | 27673 |
| HMN345096 | 227 | 748 |
| HMN345097 | 662 | 2117 |
| HMN345098 | 5347 | 617 |
| HMN345099 | 24960 | |
| HMN345100 | 1815 | 1665 |
| HMN345101 | 3334 | 7748 |
| HMN345102 | 995 | 50 |
| HMN345103 | 769 | 1384 |
| HMN345104 | 798 | 444 |
| HMN345105 | 4512 | 1686 |
| HMN345106 | | 32 |
| HMN345107 | 950 | 466 |

### Example 5: Determination of Antibody Titers in B Cell Supernatants in a Non-Competitive Microsphere Immunoassay Set-Up

Using the non-competitive microsphere immunoassay set-up according to Example 3, also B cell supernatants can be analyzed for the presence of norovirus-reactive antibodies, in particular, for the presence of specific monoclonal Abs.

The non-competitive microsphere immunoassay was carried out essentially as described under Example 3. In brief, B cell supernatants were diluted in assay buffer and 100 μL dilution were mixed with 50 μL microsphere mixture in an assay plate. The B cell supernatant and the microspheres were incubated for 90 minutes at room temperature at 600 rpm shaking. The plate was washed using a plate washer. 50 μL of an anti-rabbit IgG-PE detection antibody were added per well and the plate was incubated for 60 minutes at room temperature. After incubation, the plate was washed and 95 μL sheath fluid were added per well prior to measuring the plate.

B cell supernatants were screened for norovirus-reactive antibodies in a multiplex non-competitive assay set-up using microspheres coupled to norovirus VLPs listed in Table 16. The B cells were derived from a rabbit immunized with GII.2 OH VLPs. For instance, B cell supernatant #1 revealed a particularly high MFI value against GII.2 OH and lower values against all other VLPs. B cell Supernatant #2 in contrast shows high MFI values for most VLPs. Other supernatants show different patterns of MFI values for the VLPs indicative of antibodies with different specificities.

TABLE 16
MFI values as determined in different B cell supernatants with a multiplex non-competitive microsphere immunoassay
set-up using norovirus VLPs as described in Table 1 (e.g. GI.1 Norwalk = GI.1 VLP as listed in Table 1).

| | | | | | | | | GII.17 | | | GII.4 |
|---|---|---|---|---|---|---|---|---|---|---|---|
| | | GII. 1 | GII.2 | GII.3 | GII.6 | GII.7 | GII.12 | French | GII.17 | GII.4 | Den |
| | | Ascension | OH | USA | Ehime | ACX | AJP | Guiana | KP 2015 | CN | Haag |
| B cell | #1 | 2878 | 55889 | 901 | 1021 | 4340 | 3048 | 546 | 722 | 475 | 2717 |
| supernatant | #2 | 197358 | 44497 | 204554 | 135837 | 197967 | 186909 | 157493 | 208393 | 76327 | 169666 |
| | #3 | 158470 | 36945 | 170862 | 195760 | 150378 | 176747 | 136997 | 188173 | 34807 | 150217 |
| | #4 | 229 | 62023 | 233 | 397 | 188 | 2580 | 22601 | 244 | 145 | 201 |
| | #5 | 9620 | 53543 | 343 | 105800 | 240 | 44489 | 85762 | 195376 | 150 | 222 |
| | #6 | 231 | 51636 | 257 | 207 | 210 | 296 | 16546 | 65838 | 189 | 243 |
| | #7 | 173 | 67446 | 204 | 232 | 154 | 1829 | 162 | 209 | 158 | 162 |
| | #8 | 190 | 65263 | 193 | 240 | 175 | 237 | 144 | 213 | 170 | 175 |
| | #9 | 279 | 65033 | 414 | 479 | 261 | 400 | 259 | 293 | 164 | 245 |
| | #10 | 205234 | 48258 | 211612 | 164950 | 217184 | 215759 | 166651 | 220631 | 104858 | 183013 |

| | | | GII.4 | | | | | | | | |
|---|---|---|---|---|---|---|---|---|---|---|---|
| | | GII.4 | New | GII.4 | GI.1 | GI.2 | GI.3 | GI.4 | GI.5 | GI.6 | GI.7 |
| | | Yerseke | Orleans | Sydney | Norwalk | Jingzhou | Sweden | US | Siklos | USA | Providence |
| B cell | #1 | 2707 | 2892 | 3478 | 115 | 209 | 496 | 295 | 777 | 149 | 1620 |
| supernatant | #2 | 170874 | 170655 | 175897 | 222 | 57337 | 121088 | 7019 | 111938 | 11733 | 20346 |
| | #3 | 144366 | 136400 | 148975 | 15356 | 150274 | 184932 | 164322 | 94144 | 186449 | 469 |
| | #4 | 196 | 181 | 141 | 114 | 185 | 274 | 214 | 295 | 123 | 172 |
| | #5 | 210 | 195 | 164 | 130 | 197 | 292 | 273 | 396 | 175 | 223 |
| | #6 | 216 | 219 | 183 | 179 | 203 | 243 | 253 | 258 | 179 | 230 |
| | #7 | 148 | 174 | 180 | 114 | 173 | 174 | 192 | 196 | 149 | 168 |
| | #8 | 160 | 150 | 154 | 120 | 150 | 213 | 197 | 243 | 120 | 161 |
| | #9 | 248 | 249 | 219 | 140 | 218 | 355 | 327 | 485 | 183 | 291 |
| | #10 | 184059 | 187425 | 187609 | 163 | 15811 | 175016 | 489 | 35872 | 6578 | 405 |

#1227 IgH expression insert (SEQ ID NO: 23 = DNA sequence of variable heavy chain of
mAb 1227)
GCCACCatgtacaggatgcaactcctgtcttgcattgcactaagtcttgcacttgtcacaaacagtCAAGTACAATTG
GTGCAAAGTGGAGGTGGAATGGTACAACCCGGTGGTTCACTGAGCCTCTCT
TGCGCCGCATCTGGATTTACGCTCTCCAACTACGCCATGACATGGGTACGCC
AGGCGCCGGGGAAAGGTCTCGAATGGGTCTCCTCAATAGGTGGCTCCGGTG
GAACTACCTACTATGCCGATAGCGTTAAGGGACGCTTCACTATCAGCCGAG
ATTCCTCTATGAACACCCTGTATTTGCAAATGTCAAACCTCCGAGCGGGCGA
CACCGCAGTTTACTATTGTGCTAAGGATAAAACGCGCACTCTCCGCCTTGGC
TATAGTGGTATGGACGTGTGGGGACAGGGAACTACAGTTACAGTTAGTTCAg
cctccaccaagggcccatcggtcttccccctggcaccctcctccaagagcacctctgggggcacagcggccctgggctgcctggtca
aggactacttccccgaaccggtgacggtgtcgtggaactcaggcgccctgaccagcggcgtgcacaccttcccggctgtcctacagt
cctcaggactctactccctcagcagcgtggtgaccgtgccctccagcagcttgggcacccagacctacatctgcaacgtgaatcacaa
gcccagcaacaccaaggtggacaagaaagttgagcccaaatcttgtgacaaaactcacacatgcccaccgtgcccagcacctgaact
cctggggggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaagccgc
gggaggagcagtacaacagcacgtaccgggtggtcagcgtcctcaccgtcctgcaccaggactggctgaatggcaaggagtacaa
gtgcaaggtctccaacaaagccctcccagcccccatcgagaaaaccatctccaaagccaaagggcagccccgagaaccacaggtg
tacaccctgcccccatcccgggatgagctgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttcttcct
ctacagcaagctcaccgtggacaagagcaggtggcagcaggggaacgtcttctcatgctccgtgatgcatgaggctctgcacaacca
ctacacgcagaagagcctctccctgtctccgggtaaaTGA
Kozak seq-IL-2 signal sequence-VH-constant region human IgG1-termination TGA
#1227 IgK expression insert (SEQ ID NO: 24 = DNA sequence of variable light chain of
mAb 1227)
GCCACCatgtacaggatgcaactcctgtcttgcattgcactaagtcttgcacttgtcacaaacagtGATATACGCCTG
ACGCAAAGCCCGTCATCCCTTTCCGCTTCAGTGGGCGATAGAGTAACGATA
ACCTGTAGGGCAAGCCAATCCATAAGCTCTTACCTTAACTGGTATCAGCAGA
AACCGGGCAAGGCTCCCGACCTCCTTATATACGGGGCATCCTCACTTCAGTC
CGGCGTCCCTTCAAGATTTAGTGGGTCAGGGTCCGGCACGGACTTTACATT
GACGATATCCTCATTGCAGCCTGAGGATTTCGGCAACTACTATTGTCAGCAG
TCATTCTCAACGCCGAGAACATTTGGACAAGGTACGAAAGTTGAGCTGAaacg
aactgtggctgcaccatctgtcttcatcttcccgccatctgatgagcagttgaaatctggaactgcctctgttgtgtgcctgctgaataactt
ctatcccagagaggccaaagtacagtggaaggtggataacgccctccaatcgggtaactcccaggagagtgtcacagagcaggaca
gcaaggacagcacctacagcctcagcagcaccctgacgctgagcaaagcagactacgagaaacacaaagtctacgcctgcgaagt
cacccatcagggcctgagctcgcccgtcacaaagagcttcaacaggggagagtgtTGA
Kozak seq-IL-2 signal sequence-VL-constant region human IgK-termination TGA
#1431 IgH expression insert (SEQ ID NO: 25 = DNA sequence of variable heavy chain of
mAb 1431)
GCCACCatgtacaggatgcaactcctgtcttgcattgcactaagtcttgcacttgtcacaaacagtCAAGTGCAACTG
GTCCAGTCTGGTGGGGGCCTTGTCCAGCCAGGTGGGTCACTTAGACTTTCC
TGCGCTGCCAGCGGTTTTGCATTCAGTAATCACGGTATGCACTGGGTACGC
CAAGCGCCCGGCAAAGGCCTGGAGTGGCTCTCCTATATATCTGGTTCAACA
GGCGCGATTCATTACGCTAACTCAGTAAAAGGGAGGTTTACAATCTCAAGA
GACAACGCAAGGAATTCCCTGGATTTGCAGATGAATAGCCTGGGGGACGAG
GACACCGCAGTTTACTACTGTGCCCGCGACGGACCGCGCCCCGACGGTACG
GGCTACGCCGGCCCTTCTAACGACTATTGGGGTCAGGGTACTCTGGTCTCC
GTGAGTAGCgcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgaccgtgccctccagcagcttgggcacccagacctacatc
tgcaacgtgaatcacaagcccagcaacaccaaggtggacaagaaagttgagcccaaatcttgtgacaaaactcacacatgcccaccg
tgcccagcacctgaactcctggggggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcataat
gccaagacaaagccgcgggaggagcagtacaacagcacgtaccgggtggtcagcgtcctcaccgtcctgcaccaggactggctga
atggcaaggagtacaagtgcaaggtctccaacaaagccctcccagcccccatcgagaaaaccatctccaaagccaaagggcagcc
ccgagaaccacaggtgtacaccctgcccccatcccgggatgagctgaccaagaaccaggtcagcctgacctgcctggtcaaaggct
tctatcccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtgatgcatg
aggctctgcacaaccactacacgcagaagagcctctccctgtctccgggtaaaTGA
Kozak seq-IL-2 signal sequence-VH-constant region human IgG1-termination TGA
#1431 IgK expression insert (SEQ ID NO: 26 = DNA sequence of variable light chain of
mAb 1431)
GCCACCatgtacaggatgcaactcctgtcttgcattgcactaagtcttgcacttgtcacaaacagtGAAATCGTCCTG
ACCCAATCACCAGCATCCCTGTCTCTTAGTCCAGGCGAAAGAGCGACCCTGT
CTTGTAAGGCTTCTAGGAGTATTTCCATTTACCTCGCGTGGTATCAACAGAA
GCCGGGCCAAGCTCCGAGGCTTTTGATTTATGATGCCTCCTACCGCGCAATC
GGAATACCGGCAAGGTTTAGTGGAAGTGGTTCCGGTACTGATTTCACTTTGA
CCATATCTAACCTTGAGCCTGAAGATTTTGCGGTGTACTACTGTCAGCATCG
GAGTTCCTGGCCAGCTTTGACCTTCGGAGGGGGGACGAAGGTCGAAATTaaac
gaactgtggctgcaccatctgtcttcatcttcccgccatctgatgagcagttgaaatctggaactgcctctgttgtgtgcctgctgaataac
ttctatcccagagaggccaaagtacagtggaaggtggataacgccctccaatcgggtaactcccaggagagtgtcacagagcagga
cagcaaggacagcacctacagcctcagcagcaccctgacgctgagcaaagcagactacgagaaacacaaagtctacgcctgcgaa
gtcacccatcagggcctgagctcgcccgtcacaaagagcttcaacaggggagagtgtTGA
Kozak seq-IL-2 signal sequence-VL-constant region human IgK-termination TGA
#1227 IgG1 (SEQ ID NO: 27 = Amino acid sequence of variable heavy chain of mAb 1227)
ATMYRMQLLSCIALSLALVTNSQVQLVQSGGGMVQPGGSLSLSCAASGFTLSNYA
MTWVRQAPGKGLEWVSSIGGSGGTTYYADSVKGRFTISRDSSMNTLYLQMSNL
RAGDTAVYYCAKDKTRTLRLGYSGMDVWGQGTTVTVSSASTKGPSVFPLAPSSK
STSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQ
QGNVFSCSVMHEALHNHYTQKSLSLSPGK
Kozak-IL2 signal peptide-VH-constant region
#1227 IgK (SEQ ID NO: 28 = Amino acid sequence of variable light chain of mAb 1227)
ATMYRMQLLSCIALSLALVINSDIRLTQSPSSLSASVGDRVTITCRASQSISSYLNW
YQQKPGKAPDLLIYGASSLQSGVPSRFSGSGSGTDFTLTISSLQPEDFGNYYCQQ
SFSTPRTFGQGTKVELKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSS
PVTKSFNRGEC
Kozak-IL2 signal peptide-VK-constant region
#1431 IgG1 (SEQ ID NO: 29 = Amino acid sequence of variable heavy chain of mAb 1431)
ATMYRMQLLSCIALSLALVTNSQVQLVQSGGGLVQPGGSLRLSCAASGFAFSNHG
MHWVRQAPGKGLEWLSYISGSTGAIHYANSVKGRFTISRDNARNSLDLQMNSL
GDEDTAVYYCARDGPRPDGTGYAGPSNDYWGQGTLVSVSSASTKGPSVFPLAPS
SKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSR
WQQGNVFSCSVMHEALHNHYTQKSLSLSPGK
Kozak-IL2 signal peptide-VH-constant region
#1431 IgK (SEQ ID NO: 30 = Amino acid sequence of variable light chain of mAb 1431)
ATMYRMQLLSCIALSLALVTNSEIVLTQSPASLSLSPGERATLSCKASRSISIYLAW
YQQKPGQAPRLLIYDASYRAIGIPARFSGSGSGTDFTLTISNLEPEDFAVYYCQH
RSSWPALTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLS
SPVTKSFNRGEC
Kozak-IL2 signal peptide-VK-constant region

### Conclusion

In summary, a non-competitive and competitive microsphere immunoassay with multiple norovirus VLPs was successfully set up. Robust performance when evaluating Ab levels within a large number of different human serum samples was demonstrated.

The non-competitive microsphere immunoassay enabled rapid characterization of the immune status of a subject, including determination of IgG, IgA, and IgM levels. By application of mAbs in the competitive set-up that show for instance norovirus-blocking properties as determined in a PGM assay and/or norovirus-neutralizing properties as determined in a human intestinal enteroid (HIE) neutralization assay (Atmar et al, 2019, Comparison of Microneutralization and Histo-Blood Group Antigen-Blocking assays for functional norovirus antibody detection, J. of Infect. Dis.), antibodies with norovirus-blocking and/or norovirus-neutralizing activity in serum samples can be examined in a reliable, relatively high-throughput, cost-effective (low samples volumes), and fast way. As the microsphere immunoassays solely use mAbs and/or VLPs they overcome the complexities and throughput limitation of cell-based assays.

The assay set-ups as developed in the present application open the door for a fast analysis of samples, including those from human patients after vaccination. This assay overcomes the limitations of PGM blockade assay as it expands the coverage to any norovirus VLP, which cannot be used in the PGM assay. In addition, it provides an alternative for the cell-based neutralization assay, which, at this time, is not applicable for most norovirus strains. Moreover, both the PGM and the cell-based neutralization assay are expensive and not suitable for clinical throughput. In contrast, the developed norovirus microsphere immunoassays with the potential to multiplexing are able to determine antibodies within any kind of sample (e.g. serum, plasma, urine) from any kind of origin (e.g. human or mouse) against any kind of norovirus in a reliable, fast and cost-effective way. Therefore, the assay set-ups developed in the present application with potential to high-throughput are an attractive method for testing of clinical samples e.g. after vaccination or infection.

### NUMBERED EMBODIMENTS OF THE PRESENT DISCLOSURE

### I. Microsphere Complex

A microsphere complex comprising a microsphere coupled to a norovirus virus like particle (VLP).
1. The microsphere complex of item 1, wherein the norovirus VLP comprises the major viral capsid protein VP1 and optionally the minor viral capsid protein VP2.
2. The microsphere complex of item 2, wherein the major viral capsid protein VP1 is at least 80% or at least 85% or at least 90% or at least 95% or 100% identical to SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 or SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22.
3. The microsphere complex of item 1 or 2, wherein the norovirus VLP is selected from the group consisting of GI.1 VLP, GI.2 VLP, GI.3 VLP, GI.4 VLP, GI.5 VLP, GI.6 VLP, GI.7 VLP, GII.1 VLP, GII.2 VLP, GII.3 VLP, GII.4/Consensus VLP, GII.4/Sydney VLP, GII.4/Yerseke VLP, GII.4/New Orleans VLP, GII.4/Den Haag VLP, GII.4/Houston VLP, GII.6 VLP, GII.7 VLP, GII.12 VLP, GII.17/1978 VLP, GII.17/2014 VLP, and GII.17/2015 VLP.
4. The microsphere complex of any one of items 1 to 4, wherein the microsphere is a polystyrene microsphere.
5. The microsphere complex of any one of items 1 to 5, wherein the microsphere is magnetic.
6. The microsphere complex of any one of items 1 to 6, wherein the microsphere has a diameter in the range from about 0.01 to about 100 μm, preferably in the range from about 1 to 10 μm.
7. The microsphere complex of any one of items 1 to 7, wherein the microsphere contains carboxylate groups at the microsphere surface.
8. The microsphere complex of item 8, wherein coupling of the microsphere to the norovirus VLP occurs by formation of an amide bond between a carboxylate group of the microsphere and an amine group of the norovirus VLP.
9. The microsphere complex of any one of items 1 to 9, wherein the microsphere comprises a detectable label.
10. The microsphere complex of item 10, wherein the detectable label is at least one fluorescent dye.
11. The microsphere complex of item 11, wherein the at least one fluorescent dye is selected from the group consisting of squaraine, phthalocyanine, naphthalocyanine, and any derivative thereof.
12. The microsphere complex of item 11 or 12, wherein the microsphere can be identified by the emission signal of the at least one fluorescent dye upon irradiation with a light source.

### II. Kit

A kit comprising an amount of at least one microsphere complex of any one of items 1 to 13 and optionally an amount of a detection antibody.
13. A kit comprising:
   an amount of at least one microsphere complex of any one of items 1 to 13, and
   an amount of at least one reporter antibody that binds to the norovirus VLP of the at least one microsphere complex.
14. The kit according to item 15, wherein the at least one reporter antibody is a norovirus-neutralizing antibody.
15. The kit according to item 15 or 16, wherein the at least one reporter antibody is a norovirus-blocking antibody.
16. The kit according to any one of items 15 to 17, wherein the at least one reporter antibody is a monoclonal antibody.
17. The kit according to any one of items 15 to 18, wherein the at least one reporter antibody is derived from a non-human origin.
18. The kit according to any one of items 15 to 19, wherein the at least one reporter antibody is attached to a detectable label by the heavy chain constant region of the at least one reporter antibody.
19. The kit of item 20, wherein the at least one reporter antibody is indirectly attached to the detectable label by the heavy chain constant region of the at least one reporter antibody, wherein the reporter antibody reacts with a secondary reporter antibody directly attached to a detectable label.
20. The kit of item 21, wherein the secondary reporter antibody is directly attached to the detectable label by the heavy chain constant region of the secondary reporter antibody.
21. The kit of item 20, wherein the at least one reporter antibody is directly attached to the detectable label by the heavy chain constant region of the at least one reporter antibody.
22. The kit of any of items 20 to 23, wherein the detectable label is a fluorescence label.
23. The kit of item 24, wherein the fluorescence label is selected from the group consisting of xanthene, fluorescein isothiocyanate, rhodamine, phycoerythrin, cyanine, coumarin, and any derivative thereof.
24. The kit of item 25, wherein the detectable label is phycoerythrin.
25. The kit according to any one of items 15 to 26, wherein the at least one reporter antibody provides an EC₅₀ value towards the norovirus VLP of the at least one microsphere complex of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.03 μg/mL or less than 0.02 μg/mL or less than 0.01 μg/mL.
26. The kit according to any one of items 15 to 27, wherein the at least one reporter antibody comprises
   a heavy chain variable region (VH) amino acid sequence as represented by SEQ ID NO: 27, and
   a light chain variable region (VL) amino acid sequence as represented by SEQ ID NO: 28.
27. The kit according to any one of items 15 to 27, wherein the at least one reporter antibody comprises
   a heavy chain variable region (VH) amino acid sequence as represented by SEQ ID NO: 29, and
   a light chain variable region (VL) amino acid sequence as represented by SEQ ID NO: 30.
28. The kit according to any one of items 15 to 27, wherein the kit comprises
   an amount of two microsphere complexes according to any one of items 1 to 13,
      wherein the first microsphere complex comprises a first microsphere coupled to a first norovirus VLP and the second microsphere complex comprises a second microsphere coupled to a second norovirus VLP, and
      wherein the first microsphere comprises a first detectable label and the second microsphere comprises a second detectable label and wherein the emission signal of the first detectable label differs from the emission signal of the second detectable label, and
   and an amount of two reporter antibodies,
      wherein the first reporter antibody binds to the first norovirus VLP and does not bind to the second norovirus virus like particle, and
      wherein the second reporter antibody binds to the second norovirus VLP and does not bind to the first norovirus VLP.
29. The kit according to item 30, wherein the first or the second reporter antibody comprises
   a heavy chain variable region (VH) amino acid sequence as represented by SEQ ID NO: 27, and
   a light chain variable region (VL) amino acid sequence as represented by SEQ ID NO: 28;
   or
   a heavy chain variable region (VH) amino acid sequence as represented by SEQ ID NO: 29, and
   a light chain variable region (VL) amino acid sequence as represented by SEQ ID NO: 30.
30. The kit according to item 30, wherein the first norovirus VLP is a GI.1 VLP and the second norovirus virus like particle is a GII.4/Consensus VLP.
31. The kit according to item 32, wherein the second reporter antibody comprises
   a heavy chain variable region (VH) amino acid sequence as represented by SEQ ID NO: 29, and
   a light chain variable region (VL) amino acid sequence as represented by SEQ ID NO: 30;
   and optionally wherein the first reporter antibody comprises
   a heavy chain variable region (VH) amino acid sequence as represented by SEQ ID NO: 27, and
   a light chain variable region (VL) amino acid sequence as represented by SEQ ID NO: 28.
32. The kit according to any one of items 15 to 29, wherein the kit comprises an amount of one microsphere complex according to any one of items 1 to 13 and an amount of one reporter antibody that binds to the norovirus VLP of the microsphere complex.

### III. Method for Detecting Total Ig-Levels (Non-Competitive Assay Set-Up)

### III.1 Singleplex Assay Set-Up

33. A method for detecting a signal from a detection antibody indicative for the presence and/or amount of norovirus-reactive antibodies in a sample from a subject comprising the steps of:
   Step 1: contacting an amount of a microsphere complex according to any one of items 1 to 13 with the sample to allow binding of the norovirus-reactive antibodies in the sample to the norovirus virus like particles (VLPs) coupled to the microspheres in the microsphere complex,
   Step 2: contacting an amount of a detection antibody with the norovirus-reactive antibodies bound to the norovirus VLPs in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a detectable label, and
   Step 3: detecting a signal from the detection antibody bound to the norovirus-reactive antibodies in step 2.
34. The method according to item 35 for determining the presence and/or amount of norovirus-reactive antibodies in a sample from a subject, wherein the method comprises the further steps of:
   Step 4: determining the presence and/or amount of the detection antibody bound to the norovirus-reactive antibodies from the signal of step 3, and
   Step 5: determining the presence and/or amount of norovirus-reactive antibodies in the sample from the presence and/or amount of the detection antibody determined in step 4.

### III.2 Multiplex Assay Set-Up

35. A method for detecting a signal from a detection antibody indicative for the presence and/or amount of norovirus-reactive antibodies in a sample from a subject comprising the steps of:
   Step 1: contacting an amount of at least two microsphere complexes according to any one of items 1 to 13,
   wherein the first microsphere complex comprises a first microsphere coupled to a first norovirus VLP and the second microsphere complex comprises a second microsphere coupled to a second norovirus VLP, and
   wherein the first microsphere comprises a first detectable label and the second microsphere comprises a second detectable label and wherein the emission signal of the first detectable label differs from the emission signal of the second detectable label,
   with the sample to allow binding of the norovirus-reactive antibodies in the sample to the first and/or the second norovirus VLP,
   Step 2: contacting an amount of a detection antibody with the norovirus-reactive antibodies bound to the first and/or the second norovirus VLP in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a third detectable label,
   Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
   simultaneously detecting a signal from the detection antibody bound to the norovirus-reactive antibodies bound to the norovirus VLP of the at least one microsphere in step 2 upon irradiation with a second light source,
   Step 4: repeating step 3 until at least 30 microspheres coupled to the same norovirus VLP are identified, and
   Step 5: summarizing the detected signal from the detection antibody in step 3 for all identified microspheres coupled to the same norovirus VLP, wherein the summarized signal is indicative for the presence and/or amount of norovirus-reactive antibodies in the sample.
36. The method according to item 37 for determining the presence and/or amount of norovirus-reactive antibodies in a sample from a subject, wherein the method comprises the further steps of:
   Step 6: determining the presence and/or amount of the detection antibody bound to the norovirus-reactive antibodies from the summarized signal of step 5, and
   Step 7: determining the presence and/or amount of norovirus-reactive antibodies in the sample from the presence and/or amount of the detection antibody determined in step 6.
37. The method of item 37 or 38, wherein in step 1 an amount of at least five or at least ten or at least fifteen or at least twenty microsphere complexes is contacted with the sample.
38. The method of any one of items 37 to 39, wherein in step 1 an amount of a first microsphere complex comprising a first microsphere coupled to a GI.1 VLP, an amount of a second microsphere complex comprising a second microsphere coupled to a GI.2 VLP, an amount of a third microsphere complex comprising a third microsphere coupled to a GI.3 VLP, an amount of a fourth microsphere complex comprising a fourth microsphere coupled to GI.4 VLP, an amount of a fifth microsphere complex comprising a fifth microsphere coupled to a GI.5 VLP, an amount of a sixth microsphere complex comprising a sixth microsphere coupled to a GI.6 VLP, an amount of a seventh microsphere complex comprising a seventh microsphere coupled to a GI.7 VLP, an amount of an eight microsphere complex comprising an eight microsphere coupled to GII.1 VLP, an amount of a ninth microsphere complex comprising a ninth microsphere coupled to a GII.2 VLP, an amount of a tenth microsphere complex comprising a tenth microsphere coupled to a GII.3 VLP, an amount of an eleventh microsphere complex comprising an eleventh microsphere coupled to a GII.4/Consensus VLP, an amount of a twelfth microsphere complex comprising a twelfth microsphere coupled to GII.4/Sydney VLP, an amount of a thirteenth microsphere complex comprising a thirteenth microsphere coupled to a GII.4/New Orleans VLP, an amount of a fourteenth microsphere complex comprising a fourteenth microsphere coupled to a GII.4/Yerseke VLP, an amount of a fifteenth microsphere complex comprising a fifteenth microsphere coupled to a GII.4/Den Haag VLP, an amount of a sixteenth microsphere complex comprising a sixteenth microsphere coupled to GII.6 VLP, an amount of a seventeenth microsphere complex comprising a seventeenth microsphere coupled to a GII.7 VLP, an amount of an eighteenth microsphere complex comprising an eighteenth microsphere coupled to a GII.12 VLP, an amount of a nineteenth microsphere complex comprising a nineteenth microsphere coupled to a GII.17/1978 VLP, and an amount of a twentieth microsphere complex comprising a twentieth microsphere coupled to GII.17/2015 VLP is contacted with the sample.
39. The method according to any one of items 35 to 40, wherein the detection antibody is directly attached to the detectable label by the heavy chain constant region of the detection antibody.
40. The method according to any one of items 35 to 41, wherein the detectable label the detection antibody is attached to is a fluorescence label.
41. The method of item 42, wherein the fluorescence label is selected from the group consisting of xanthene, fluorescein isothiocyanate, rhodamine, phycoerythrin, cyanine, coumarin, and any derivative thereof.
42. The method of item 43, wherein the fluorescence label is phycoerythrin.
43. The method according to any one of items 35 to 44, wherein the signal from the detection antibody in step 3 is resulting from the detectable label the detection antibody is attached to.
44. The method according to any one of items 35 to 45, wherein contacting in step 1 is carried out for about 1 to about 24 hours.
45. The method according to item 46, wherein contacting in step 1 is carried out for about 90 minutes.
46. The method according to item 46, wherein contacting in step 1 is carried out for about 18 to about 24 hours, preferably for about 21 hours.
47. The method according to any one of items 35 to 48, wherein contacting in step 1 is carried out at a temperature of about 2 to about 30° C.
48. The method according to item 49, wherein contacting in step 1 is carried out at a temperature of about 22° C.
49. The method according to item 49, wherein contacting in step 1 is carried out at a temperature of about 2 to about 8° C.
50. The method according to any one of items 35 to 51, wherein contacting in step 2 is carried out for about 30 to about 90 minutes, preferably for about 60 minutes.
51. The method according to any one of items 35 to 52, wherein the detection antibody is derived from a non-human origin.
52. The method according to any one of items 35 to 53, wherein the detection antibody binds to antibodies from the isotype A (IgA) and does not bind to antibodies from other isotypes.
53. The method according to any one of items 35 to 53, wherein the detection antibody binds to antibodies from the isotype G (IgG) and does not bind to antibodies from other isotypes.
54. The method according to any one of items 35 to 53, wherein the detection antibody binds to antibodies from the isotype M (IgM) and does not bind to antibodies from other isotypes.
55. The method according to any one of items 35 to 53, wherein the detection antibody binds to antibodies from the isotype A, G, and M (IgA, IgG, and IgM).

### IV. Method for Determining Specific Ig-Levels (Competitive Assay Set-Up)

### IV.1 Singleplex Assay Set-Up

56. A method for detecting a signal from a reporter antibody indicative for the presence and/or amount of norovirus-reactive antibodies in a sample from a subject comprising the steps of:
   Step 1: providing a kit according to item 34, including an amount of a microsphere complex and an amount of a reporter antibody,
   Step 2: contacting the amount of the microsphere complex and the amount of the reporter antibody with the sample to allow binding of the norovirus-reactive antibodies in the sample to the norovirus VLPs coupled to the microspheres in the microsphere complex while competing with the reporter antibody, and
   Step 3: detecting a signal from the reporter antibody bound to the norovirus VLPs in step 2.
57. The method according to item 58, wherein in step 2 the amount of the microsphere complex and the amount of the reporter antibody are concomitantly contacted with the sample.
58. The method according to item 58, comprising the steps of:
   Step 1: providing a kit according to item 34, including an amount of a microsphere complex and an amount of a reporter antibody,
   Step 2.1: contacting the amount of the microsphere complex of step 1 with the sample to allow binding of norovirus-reactive antibodies in the sample to the norovirus VLPs coupled to the microspheres in the microsphere complex,
   Step 2.2: contacting the amount of the reporter antibody with the microsphere complex and the sample of step 2.1 to allow binding of the reporter antibody to the norovirus VLPs coupled to the microspheres in the microsphere complex, and
   Step 3: detecting a signal from the reporter antibody bound to the norovirus VLPs in step 2.2.
59. The method according to item 58, comprising the steps of:
   Step 1: providing a kit according to item 34, including an amount of a microsphere complex and an amount of a reporter antibody,
   Step 2.1: contacting the amount of the microsphere complex of step 1 with the sample to allow binding of norovirus-reactive antibodies in the sample to the norovirus VLPs coupled to the microspheres in the microsphere complex,
   Step 2.2: contacting the amount of the reporter antibody with the microsphere complex and the sample of step 2.1 to allow binding of the reporter antibody to the norovirus VLPs coupled to the microspheres in the microsphere complex,
   Step 2.3: contacting the amount of reporter antibody, the amount of microsphere complex, and the sample of step 2.2 with an amount of a secondary reporter antibody to allow binding of the secondary reporter antibody to the constant region of the reporter antibody, and
   Step 3: detecting a signal from the secondary reporter antibody bound to the reporter antibody in step 2.3, wherein the reporter antibody is bound to the norovirus VLPs in step 2.2.
60. The method according to any one of items 58 to 61 for determining the presence and/or amount of norovirus-reactive antibodies in a sample from a subject, wherein the method comprises the further steps of:
   Step 4: determining the presence and/or amount of the reporter antibody from the signal of step 3, and
   Step 5: determining the presence and/or amount of norovirus-reactive antibodies in the sample from the presence and/or amount of the reporter antibody determined in step 4.
61. The method according to any one of items 58 to 62, wherein the norovirus VLP is a GII.4/Sydney VLP.

### IV.2 Multiplex Assay Set-Up

62. A method for detecting a signal from a reporter antibody indicative for the presence and/or amount of norovirus-reactive antibodies in a sample from a subject comprising the steps of:
   Step 1: providing a kit according to any one of items 15 to 33, including an amount of at least two microsphere complexes,
   wherein the first microsphere complex comprises a first microsphere coupled to a first norovirus VLP and the second microsphere complex comprises a second microsphere coupled to a second norovirus VLP, and
   wherein the first microsphere comprises a first detectable label and the second microsphere comprises a second detectable label and wherein the emission signal of the first detectable label differs from the emission signal of the second detectable label, and
   and an amount of at least two reporter antibodies,
   wherein the first reporter antibody binds to the first norovirus VLP and does not bind to the second norovirus virus like particle, and
   wherein the second reporter antibody binds to the second norovirus VLP and does not bind to the first norovirus VLP;
   Step 2: contacting the amount of the at least two microsphere complexes with the sample to allow binding of the norovirus-reactive antibodies in the sample to the first and/or the second norovirus VLPs while competing with the at least two reporter antibodies;
   Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
   simultaneously detecting a signal from the reporter antibody bound to the norovirus VLPs of the at least one microsphere in step 2 upon irradiation with a second light source;
   Step 4: repeating step 3 until at least 30 microspheres coupled to the same norovirus VLP are identified; and
   Step 5: summarizing the detected signal from the reporter antibody in step 3 for all identified microspheres coupled to the same norovirus VLP, wherein the summarized signal is indicative for the presence and/or amount of norovirus-reactive antibodies in the sample.
63. The method according to item 64, wherein in step 2 the amount of the at least two microsphere complexes and the amount of the at least two reporter antibodies are concomitantly contacted with the sample.
64. The method of item 64, comprising the steps of:
   Step 1: providing a kit according to any one of items 15 to 33, including an amount of at least two microsphere complexes,
   wherein the first microsphere complex comprises a first microsphere coupled to a first norovirus VLP and the second microsphere complex comprises a second microsphere coupled to a second norovirus VLP, and
   wherein the first microsphere comprises a first detectable label and the second microsphere comprises a second detectable label and wherein the emission signal of the first detectable label differs from the emission signal of the second detectable label, and
   and an amount of at least two reporter antibodies,
   wherein the first reporter antibody binds to the first norovirus VLP and does not bind to the second norovirus virus like particle, and
   wherein the second reporter antibody binds to the second norovirus VLP and does not bind to the first norovirus VLP;
   Step 2.1: contacting the amount of the at least two microsphere complexes with the sample to allow binding of the norovirus-reactive antibodies in the sample to the first and/or the second norovirus VLPs;
   Step 2.2: contacting the amount of the at least two reporter antibodies with the at least two microsphere complexes and the sample of step 2.1 to allow binding of the at least two reporter antibodies to the norovirus VLPs coupled to the microspheres;
   Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
   simultaneously detecting a signal from the reporter antibody bound to the norovirus VLPs of the at least one microsphere in step 2.2 upon irradiation with a second light source;
   Step 4: repeating step 3 until at least 30 microspheres coupled to the same norovirus VLP are identified; and
   Step 5: summarizing the detected signal from the reporter antibody in step 3 for all identified microspheres coupled to the same norovirus VLP, wherein the summarized signal is indicative for the presence and/or amount of norovirus-reactive antibodies in the sample.
65. The method of item 64, comprising the steps of:
   Step 1: providing a kit according to any one of items 15 to 33, including an amount of at least two microsphere complexes,
   wherein the first microsphere complex comprises a first microsphere coupled to a first norovirus VLP and the second microsphere complex comprises a second microsphere coupled to a second norovirus VLP, and
   wherein the first microsphere comprises a first detectable label and the second microsphere comprises a second detectable label and wherein the emission signal of the first detectable label differs from the emission signal of the second detectable label, and
   and an amount of at least two reporter antibodies,
   wherein the first reporter antibody binds to the first norovirus VLP and does not bind to the second norovirus virus like particle, and
   wherein the second reporter antibody binds to the second norovirus VLP and does not bind to the first norovirus VLP;
   Step 2.1: contacting the amount of the at least two microsphere complexes with the sample to allow binding of the norovirus-reactive antibodies in the sample to the first and/or the second norovirus VLPs;
   Step 2.2: contacting the amount of the at least two reporter antibodies with the at least two microsphere complexes and the sample of step 2.1 to allow binding of the at least two reporter antibodies to the norovirus VLPs coupled to the microspheres;
   Step 2.3: contacting the amount of the at least two reporter antibodies, the amount of the at least two microsphere complexes and the sample of step 2.2 with an amount of a secondary reporter antibody to allow binding of the secondary reporter antibody to the constant region of the at least two reporter antibodies;
   Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
   simultaneously detecting a signal from the secondary reporter antibody bound to the reporter antibody bound to the norovirus VLPs of the at least one microsphere in step 2.3 upon irradiation with a second light source;
   Step 4: repeating step 3 until at least 30 microspheres coupled to the same norovirus VLP are identified; and
   Step 5: summarizing the detected signal from the secondary reporter antibody in step 3 for all identified microspheres coupled to the same norovirus VLP, wherein the summarized signal is indicative for the presence and/or amount of norovirus-reactive antibodies in the sample.
66. The method according to items 64 to 67, wherein the method comprises the further steps of:
   Step 6: determining the presence and/or amount of the reporter antibody from the summarized signal of step 5, and
   Step 7: determining the presence and/or amount of norovirus-reactive antibodies in the sample from the presence and/or amount of the reporter antibody determined in step 6.
67. The method according to any one of items 64 to 68, wherein the kit in step 1 provides an amount of two microsphere complexes and an amount of two reporter antibodies.
68. The method of item 69, wherein the first microsphere complex comprises a first microsphere coupled to a GI.1 VLP and wherein the second microsphere complex comprises a second microsphere coupled to a GII.4/Consensus VLP.
69. The method according to any one of items 58 to 70, wherein the signal in step 3 is resulting from the detectable label the reporter antibody is attached to.
70. The method according to any one of items 60 to 63 and 66 to 71, wherein contacting in step 2.1 is carried out for about 5 to about 23 hours.
71. The method according to item 72, wherein contacting in step 2.1 is carried out for about 8 to about 21 hours, preferably for about 16 hours.
72. The method according to item 72 or 73, wherein contacting in step 2.1 is carried out at a temperature of about 2 to about 30° C.
73. The method according to item 74, wherein contacting in step 2.1 is carried out at a temperature of about 4° C.
74. The method according to any one of items 60 to 63 and 66 to 75, wherein contacting in step 2.2 is carried out for about 10 to about 90 minutes, preferably for about 60 minutes.
75. The method according to item 76, wherein contacting in step 2.2 is carried out at about 22° C.
76. The method according to any one of items 61 to 63 and 67 to 77, wherein contacting in step 2.3 is carried out for about 10 to about 90 minutes, preferably for about 60 minutes.
77. The method according to item 78, wherein contacting in step 2.3 is carried out at about 22° C.

### V. Method for Diagnosing

A method for diagnosing a norovirus infection in a subject comprising the steps of:
Step 1: providing a sample from the subject outside the subject body,
Step 2: determining the amount of norovirus-reactive antibodies in the sample according to any one of items 35 to 79, and
Step 3: determining infection by comparing the amount of norovirus-reactive antibodies to established amounts of norovirus-reactive antibodies in norovirus infected subjects.
78. The method according to item 80, wherein the subject is infected by at least two different noroviruses.
79. The method according to item 80 or 81, wherein the norovirus infection is acute or convalescent.

### VI. Method for Determining Protection

A method for determining protection of a subject against a norovirus infection comprising the steps of:
Step 1: providing a sample from the subject outside the subject body,
Step 2: determining the amount of norovirus-reactive antibodies in the sample according to any one of items 35 to 79, and
Step 3: determining protection by comparing the amount of norovirus-reactive antibodies in step 2 to protective amounts of norovirus-reactive antibodies.
80. The method according to item 83, wherein the subject is vaccinated with a norovirus vaccine.
81. The method of item 83 or 84, wherein the norovirus-reactive antibodies are norovirus-neutralizing antibodies.

### VII. Specification of Subject and Sample

The method according to any one of items 35 to 85, wherein the sample is selected from the group consisting of blood, urine, saliva, cerebrospinal fluid, and lymph fluid.
82. The method according to item 86, wherein the sample is a serum or a blood plasma sample.
83. The method according to item 86 or 87, wherein the sample is heat-inactivated.
84. The method according to any one of items 35 to 88, wherein the subject is a mammal, preferably the mammal is selected from the group consisting of mouse, primate, non-human primate, human, rabbit, cat, rat, horse, and sheep.
85. The method according to item 89, wherein the subject is a human.
86. The method according to item 90, wherein the subject is a newborn up to 2 months of age or a child, the child being 2 months to 5 years of age.

What is claimed is:

1. A microsphere complex comprising a microsphere coupled to a norovirus virus like particle (VLP).

2. The microsphere complex of claim 1, wherein the norovirus VLP comprises the major viral capsid protein VP1 and optionally the minor viral capsid protein VP2.

3. The microsphere complex of claim 2, wherein the major viral capsid protein VP1 is at least 80% or at least 85% or at least 90% or at least 95% or 100% identical to SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 or SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22.

4. The microsphere complex of any one of claims 1 to 3, wherein the microsphere comprises a detectable label.

5. The microsphere complex of claim 4, wherein the detectable label is at least one fluorescent dye.

6. The microsphere complex of claim 5, wherein the microsphere can be identified by the emission signal of the at least one fluorescent dye upon irradiation with a light source.

7. A kit comprising an amount of at least one microsphere complex of any one of claims 1 to 6 and optionally an amount of a detection antibody.

8. A kit comprising:
an amount of at least one microsphere complex of any one of claims 1 to 6, and
an amount of at least one reporter antibody that binds to the norovirus VLP of the at least one microsphere complex.

9. The kit according to claim 8, wherein the at least one reporter antibody is attached to a detectable label by the heavy chain constant region of the at least one reporter antibody.

10. The kit of claim 9, wherein the at least one reporter antibody is indirectly attached to the detectable label by the heavy chain constant region of the at least one reporter antibody, wherein the reporter antibody reacts with a secondary reporter antibody directly attached to a detectable label.

11. The kit of claims 9 and 10, wherein the detectable label is a fluorescence label, preferably wherein the fluorescence label is selected from the group consisting of xanthene, fluorescein isothiocyanate, rhodamine, phycoerythrin, cyanine, coumarin, and any derivative thereof.

12. The kit according to any one of claims 8 to 11, wherein the at least one reporter antibody provides an EC₅₀ value towards the norovirus VLP of the at least one microsphere complex of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.03 μg/mL or less than 0.02 μg/mL or less than 0.01 μg/mL.

13. The kit according to any one of claims 8 to 12, wherein the at least one reporter antibody comprises
a heavy chain variable region (VH) amino acid sequence as represented by SEQ ID NO: 27, and
a light chain variable region (VL) amino acid sequence as represented by SEQ ID NO: 28.

14. The kit according to any one of claims 8 to 12, wherein the at least one reporter antibody comprises
a heavy chain variable region (VH) amino acid sequence as represented by SEQ ID NO: 29, and
a light chain variable region (VL) amino acid sequence as represented by SEQ ID NO: 30.

15. The kit according to any one of claims 8 to 12, wherein the kit comprises
an amount of two microsphere complexes according to any one of claims 1 to 6,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of two reporter antibodies,
wherein the first reporter antibody binds to the first norovirus VLP and does not bind to the second norovirus virus like particle, and
wherein the second reporter antibody binds to the second norovirus VLP and does not bind to the first norovirus VLP.

16. The kit according to claim 15, wherein the first or the second reporter antibody comprises
a heavy chain variable region (VH) amino acid sequence as represented by SEQ ID NO: 27, and
a light chain variable region (VL) amino acid sequence as represented by SEQ ID NO: 28;
or
a heavy chain variable region (VH) amino acid sequence as represented by SEQ ID NO: 29, and
a light chain variable region (VL) amino acid sequence as represented by SEQ ID NO: 30.

17. The kit according to claim 15, wherein the first norovirus VLP is a GI.1 VLP and the second norovirus virus like particle is a GII.4/Consensus VLP.

18. The kit according to claim 17, wherein the second reporter antibody comprises
a heavy chain variable region (VH) amino acid sequence as represented by SEQ ID NO: 29, and
a light chain variable region (VL) amino acid sequence as represented by SEQ ID NO: 30;
and optionally wherein the first reporter antibody comprises
a heavy chain variable region (VH) amino acid sequence as represented by SEQ ID NO: 27, and
a light chain variable region (VL) amino acid sequence as represented by SEQ ID NO: 28.

19. The kit according to any one of claims 8 to 14, wherein the kit comprises an amount of one microsphere complex according to any one of claims 1 to 6 and an amount of one reporter antibody that binds to the norovirus VLP of the microsphere complex.

20. A method for detecting a signal from a detection antibody indicative for the presence and/or amount of norovirus-reactive antibodies in a sample from a subject comprising the steps of:
Step 1: contacting an amount of a microsphere complex according to any one of claims 1 to 6 with the sample to allow binding of the norovirus-reactive antibodies in the sample to the norovirus virus like particles (VLPs) coupled to the microspheres in the microsphere complex,
Step 2: contacting an amount of a detection antibody with the norovirus-reactive antibodies bound to the norovirus VLPs in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a detectable label, and
Step 3: detecting a signal from the detection antibody bound to the norovirus-reactive antibodies in step 2.

21. The method according to claim 20 for determining the presence and/or amount of norovirus-reactive antibodies in a sample from a subject, wherein the method comprises the further steps of:
Step 4: determining the presence and/or amount of the detection antibody bound to the norovirus-reactive antibodies from the signal of step 3, and
Step 5: determining the presence and/or amount of norovirus-reactive antibodies in the sample from the presence and/or amount of the detection antibody determined in step 4.

22. A method for detecting a signal from a detection antibody indicative for the presence and/or amount of norovirus-reactive antibodies in a sample from a subject comprising the steps of:
Step 1: contacting an amount of at least two microsphere complexes according to any one of claims 1 to 6,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label,
with the sample to allow binding of the norovirus-reactive antibodies in the sample to the first and/or the second norovirus VLP,
Step 2: contacting an amount of a detection antibody with the norovirus-reactive antibodies bound to the first and/or the second norovirus VLP in step 1 to allow binding of the detection antibody to the heavy chain constant region of the norovirus-reactive antibodies, wherein the detection antibody binds to the norovirus-reactive antibodies with the variable region of the detection antibody and wherein the detection antibody is attached to a third detectable label,
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the detection antibody bound to the norovirus-reactive antibodies bound to the norovirus VLP of the at least one microsphere in step 2 upon irradiation with a second light source,
Step 4: repeating step 3 until at least 30 microspheres coupled to the same norovirus VLP are identified, and
Step 5: summarizing the detected signal from the detection antibody in step 3 for all identified microspheres coupled to the same norovirus VLP, wherein the summarized signal is indicative for the presence and/or amount of norovirus-reactive antibodies in the sample.

23. The method according to claim 22 for determining the presence and/or amount of norovirus-reactive antibodies in a sample from a subject, wherein the method comprises the further steps of:
Step 6: determining the presence and/or amount of the detection antibody bound to the norovirus-reactive antibodies from the summarized signal of step 5, and
Step 7: determining the presence and/or amount of norovirus-reactive antibodies in the sample from the presence and/or amount of the detection antibody determined in step 6.

24. The method of claim 22 or 23, wherein in step 1 an amount of at least five or at least ten or at least fifteen or at least twenty microsphere complexes is contacted with the sample.

25. The method of any one of claims 22 to 24, wherein in step 1 an amount of a first microsphere complex comprising a first microsphere coupled to a GI.1 VLP, an amount of a second microsphere complex comprising a second microsphere coupled to a GI.2 VLP, an amount of a third microsphere complex comprising a third microsphere coupled to a GI.3 VLP, an amount of a fourth microsphere complex comprising a fourth microsphere coupled to GI.4 VLP, an amount of a fifth microsphere complex comprising a fifth microsphere coupled to a GI.5 VLP, an amount of a sixth microsphere complex comprising a sixth microsphere coupled to a GI.6 VLP, an amount of a seventh microsphere complex comprising a seventh microsphere coupled to a GI.7 VLP, an amount of an eight microsphere complex comprising an eight microsphere coupled to GII.1 VLP, an amount of a ninth microsphere complex comprising a ninth microsphere coupled to a GII.2 VLP, an amount of a tenth microsphere complex comprising a tenth microsphere coupled to a GII.3 VLP, an amount of an eleventh microsphere complex comprising an eleventh microsphere coupled to a GII.4/Consensus VLP, an amount of a twelfth microsphere complex comprising a twelfth microsphere coupled to GII.4/Sydney VLP, an amount of a thirteenth microsphere complex comprising a thirteenth microsphere coupled to a GII.4/New Orleans VLP, an amount of a fourteenth microsphere complex comprising a fourteenth microsphere coupled to a GII.4/Yerseke VLP, an amount of a fifteenth microsphere complex comprising a fifteenth microsphere coupled to a GII.4/Den Haag VLP, an amount of a sixteenth microsphere complex comprising a sixteenth microsphere coupled to GII.6 VLP, an amount of a seventeenth microsphere complex comprising a seventeenth microsphere coupled to a GII.7 VLP, an amount of an eighteenth microsphere complex comprising an eighteenth microsphere coupled to a GII.12 VLP, an amount of a nineteenth microsphere complex comprising a nineteenth microsphere coupled to a GII.17/1978 VLP, and an amount of a twentieth microsphere complex comprising a twentieth microsphere coupled to GII.17/2015 VLP is contacted with the sample.

26. The method according to any one of claims 20 to 25, wherein the detection antibody is directly attached to the detectable label by the heavy chain constant region of the detection antibody.

27. The method according to any one of claims 20 to 26, wherein the detectable label the detection antibody is attached to is a fluorescence label, preferably wherein the fluorescence label is selected from the group consisting of xanthene, fluorescein isothiocyanate, rhodamine, phycoerythrin, cyanine, coumarin, and any derivative thereof.

28. A method for detecting a signal from a reporter antibody indicative for the presence and/or amount of norovirus-reactive antibodies in a sample from a subject comprising the steps of:
Step 1: providing a kit according to claim 19, including an amount of a microsphere complex and an amount of a reporter antibody,
Step 2: contacting the amount of the microsphere complex and the amount of the reporter antibody with the sample to allow binding of the norovirus-reactive antibodies in the sample to the norovirus VLPs coupled to the microspheres in the microsphere complex while competing with the reporter antibody, and
Step 3: detecting a signal from the reporter antibody bound to the norovirus VLPs in step 2.

29. The method according to claim 28, comprising the steps of:
Step 1: providing a kit according to claim 19, including an amount of a microsphere complex and an amount of a reporter antibody,
Step 2.1: contacting the amount of the microsphere complex of step 1 with the sample to allow binding of norovirus-reactive antibodies in the sample to the norovirus VLPs coupled to the microspheres in the microsphere complex,
Step 2.2: contacting the amount of the reporter antibody with the microsphere complex and the sample of step 2.1 to allow binding of the reporter antibody to the norovirus VLPs coupled to the microspheres in the microsphere complex, and
Step 3: detecting a signal from the reporter antibody bound to the norovirus VLPs in step 2.2.

30. The method according to claim 28, comprising the steps of:
Step 1: providing a kit according to claim 19, including an amount of a microsphere complex and an amount of a reporter antibody,
Step 2.1: contacting the amount of the microsphere complex of step 1 with the sample to allow binding of norovirus-reactive antibodies in the sample to the norovirus VLPs coupled to the microspheres in the microsphere complex,
Step 2.2: contacting the amount of the reporter antibody with the microsphere complex and the sample of step 2.1 to allow binding of the reporter antibody to the norovirus VLPs coupled to the microspheres in the microsphere complex,
Step 2.3: contacting the amount of reporter antibody, the amount of microsphere complex, and the sample of step 2.2 with an amount of a secondary reporter antibody to allow binding of the secondary reporter antibody to the constant region of the reporter antibody, and
Step 3: detecting a signal from the secondary reporter antibody bound to the reporter antibody in step 2.3, wherein the reporter antibody is bound to the norovirus VLPs in step 2.2.

31. The method according to any one of claims 28 to 30 for determining the presence and/or amount of norovirus-reactive antibodies in a sample from a subject, wherein the method comprises the further steps of:
Step 4: determining the presence and/or amount of the reporter antibody from the signal of step 3, and
Step 5: determining the presence and/or amount of norovirus-reactive antibodies in the sample from the presence and/or amount of the reporter antibody determined in step 4.

32. A method for detecting a signal from a reporter antibody indicative for the presence and/or amount of norovirus-reactive antibodies in a sample from a subject comprising the steps of:
Step 1: providing a kit according to any one of claims 8 to 18, including an amount of at least two microsphere complexes,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies,
wherein the first reporter antibody binds to the first norovirus VLP and does not bind to the second norovirus virus like particle, and
wherein the second reporter antibody binds to the second norovirus VLP and does not bind to the first norovirus VLP;
Step 2: contacting the amount of the at least two microsphere complexes with the sample to allow binding of the norovirus-reactive antibodies in the sample to the first and/or the second norovirus VLPs while competing with the at least two reporter antibodies;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the reporter antibody bound to the norovirus VLPs of the at least one microsphere in step 2 upon irradiation with a second light source;
Step 4: repeating step 3 until at least 30 microspheres coupled to the same norovirus VLP are identified; and
Step 5: summarizing the detected signal from the reporter antibody in step 3 for all identified microspheres coupled to the same norovirus VLP, wherein the summarized signal is indicative for the presence and/or amount of norovirus-reactive antibodies in the sample.

33. The method of claim 32, comprising the steps of:
Step 1: providing a kit according to any one of claims 8 to 18, including an amount of at least two microsphere complexes,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies,
wherein the first reporter antibody binds to the first norovirus VLP and does not bind to the second norovirus virus like particle, and
wherein the second reporter antibody binds to the second norovirus VLP and does not bind to the first norovirus VLP;
Step 2.1: contacting the amount of the at least two microsphere complexes with the sample to allow binding of the norovirus-reactive antibodies in the sample to the first and/or the second norovirus VLPs;
Step 2.2: contacting the amount of the at least two reporter antibodies with the at least two microsphere complexes and the sample of step 2.1 to allow binding of the at least two reporter antibodies to the norovirus VLPs coupled to the microspheres;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the reporter antibody bound to the norovirus VLPs of the at least one microsphere in step 2.2 upon irradiation with a second light source;
Step 4: repeating step 3 until at least 30 microspheres coupled to the same norovirus VLP are identified; and
Step 5: summarizing the detected signal from the reporter antibody in step 3 for all identified microspheres coupled to the same norovirus VLP, wherein the summarized signal is indicative for the presence and/or amount of norovirus-reactive antibodies in the sample.

34. The method of claim 32, comprising the steps of:
Step 1: providing a kit according to any one of claims 8 to 18, including an amount of at least two microsphere complexes,
wherein the first microsphere complex comprises a first microsphere coupled to a first norovirus VLP and the second microsphere complex comprises a second microsphere coupled to a second norovirus VLP, and
wherein the first microsphere comprises a first detectable label and the second microsphere comprises a second detectable label and wherein the emission signal of the first detectable label differs from the emission signal of the second detectable label, and
and an amount of at least two reporter antibodies,
wherein the first reporter antibody binds to the first norovirus VLP and does not bind to the second norovirus virus like particle, and
wherein the second reporter antibody binds to the second norovirus VLP and does not bind to the first norovirus VLP;
Step 2.1: contacting the amount of the at least two microsphere complexes with the sample to allow binding of the norovirus-reactive antibodies in the sample to the first and/or the second norovirus VLPs;
Step 2.2: contacting the amount of the at least two reporter antibodies with the at least two microsphere complexes and the sample of step 2.1 to allow binding of the at least two reporter antibodies to the norovirus VLPs coupled to the microspheres;
Step 2.3: contacting the amount of the at least two reporter antibodies, the amount of the at least two microsphere complexes and the sample of step 2.2 with an amount of a secondary reporter antibody to allow binding of the secondary reporter antibody to the constant region of the at least two reporter antibodies;
Step 3: detecting the emission signal of the detectable label of at least one microsphere upon irradiation with a first light source and comparing the emission signal with the emission signal of the first detectable label and with the emission signal of the second detectable label, thereby identifying the at least one microsphere and the norovirus VLP the at least one microsphere is coupled to, and
simultaneously detecting a signal from the secondary reporter antibody bound to the reporter antibody bound to the norovirus VLPs of the at least one microsphere in step 2.3 upon irradiation with a second light source;
Step 4: repeating step 3 until at least 30 microspheres coupled to the same norovirus VLP are identified; and
Step 5: summarizing the detected signal from the secondary reporter antibody in step 3 for all identified microspheres coupled to the same norovirus VLP, wherein the summarized signal is indicative for the presence and/or amount of norovirus-reactive antibodies in the sample.

35. The method according to any one of claims 32 to 34, wherein the method comprises the further steps of:
Step 6: determining the presence and/or amount of the reporter antibody from the summarized signal of step 5, and
Step 7: determining the presence and/or amount of norovirus-reactive antibodies in the sample from the presence and/or amount of the reporter antibody determined in step 6.

36. The method according to any one of claims 32 to 35, wherein the kit in step 1 provides an amount of two microsphere complexes and an amount of two reporter antibodies.

37. The method of claim 36, wherein the first microsphere complex comprises a first microsphere coupled to a GI.1 VLP and wherein the second microsphere complex comprises a second microsphere coupled to a GII.4/Consensus VLP.

38. A method for diagnosing a norovirus infection in a subject comprising the steps of:
Step 1: providing a sample from the subject outside the subject body,
Step 2: determining the amount of norovirus-reactive antibodies in the sample according to any one of claims 20 to 37, and
Step 3: determining infection by comparing the amount of norovirus-reactive antibodies to established amounts of norovirus-reactive antibodies in norovirus infected subjects.

39. A method for determining protection of a subject against a norovirus infection comprising the steps of:
Step 1: providing a sample from the subject outside the subject body,
Step 2: determining the amount of norovirus-reactive antibodies in the sample according to any one of claims 20 to 37, and
Step 3: determining protection by comparing the amount of norovirus-reactive antibodies in step 2 to protective amounts of norovirus-reactive antibodies.

40. The method according to any one of claims 20 to 39, wherein the subject is a human.


SEQUENCE LISTING (30 sequences)

SEQ ID NO: 1 - protein, 530 aa, Norovirus norwalk virus
  source 1..530: note = strain Hu/GI.1/Norwalk/1968/US
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 2 - protein, 546 aa, Norovirus norwalk virus
  source 1..546: note = strain Hu/GI.2/Jingzhou/2013401/CHN
MMMASKDAPQSADGASGAGQLVPEVNTADPLPMEPVAGPTTAVATAGQVNMIDPWIVNNFVQSPQGEFTISPNNTPGDILFDLQLGPHLNPFLSHLSQMYNGWVGNMRVRILLAGNAFSAGKIIVCFVPPGFTSSSLTIAQATLFPHVIADVRTLEPIEMPLEDVRNVLYHTNDNQPTMRLVCMLYTPLRTGGGSGSSDSFVVAGRVLTAPSSDFSFLFLVPPTIEQKTRAFTVPNIPLQTLSNSRFPSLIQGMILSPDASQVVQFQNGRCLIDGQLLGTTPATSGQLFRVRGKINQGARTLNLTEVDGKPFMAFDSPAPVGFPDFGKCDWHMRISKTPNNTSSGDPMRSVSVQTNVQGFVPHLGSIQFDEVFNHPTGDYIGTIEWISQPSTPPGTDINLWEIPDYGSSLSQAANLAPPVFPPGFGEALVYFVSAFPGPNNRSAPNDVPCLLPQEYITHFVSEQAPTMGDAALLHYVDPDTNRNLGEFKLYPGGYLTCVPNGVGAGPQQLPLNGVFLFVSWVSRFYQLKPVGTASTARGRLGVRRI

SEQ ID NO: 3 - protein, 545 aa, Norovirus norwalk virus
  source 1..545: note = strain Hu/GI.3/JKPG_883/SWE/2007
MMMASKDAPTNMDGTSGAGQLVPEANTAEPISMEPVAGAATAAATAGQVNMIDPWIMNNYVQAPQGEFTISPNNTPGDVLFDLQLGPHLNPFLSHLAQMYNGWVGNMKVKVLLAGNAFTAGKIIISCIPPGFVAQNFSIAQATMFPHVIADVRVLEPIEVPLEDVRNVLFHNNDNTPTMRLVCMLYTPLRAGGSSSGTDPFVIAGRVLTCPSPDFSFLFLVPPNVEQKTKPFSVPNLPLNTLSNSRVPSLIKSMMISRDHGQMVQFQNGRVTLDGQLQGTTPTSASQLCKIRGSVFHANGGNGYNLTELDGSPYHAFESPAPIGFPDLGECDWHMEASPTTQFDTGDVIKQINVKQESAFAPHLGTVQADGLSDVSANTNMIAKLGWVSPVSDGHRSDVDPWVIPRYGSTLTEAAQLAPPIYPPGFGEAIVFFMSDFPIAHGTNGLSVPCTIPQEFVTHFVNEQAPTRGEAALLHYLDPDTHRNLGEFKLYPDGFMTCVPNSSGTGPQTLPINGVFVFVSWVSRFYQLKPVGTAGPARRLGIRRS

SEQ ID NO: 4 - protein, 544 aa, Norovirus norwalk virus
  source 1..544: note = strain Hu/GI.4/1643/2008/US
MMMASKDATPSADGATGAGQLVPEVNTADPIPIDPVAGSSTALATAGQVNLIDPWIINNFVQAPQGEFTISPNNTPGDVLFDLQLGPHLNPFLSHLSQMYNGWVGNMRVRVVLAGNAFTAGKVIICCVPPGFQSRTLSIAQATLFPHVIADVRTLDPVEVPLEDVRNVLYHNNDTQPTMRLLCMLYTPLRTGGASGGTDSFVVAGRVLTCPGPDFNFLFLVPPTVEQKTRPFTVPNIPLKYLSNSRIPNPIEGMSLSPDQTQNVQFQNGRCTIDGQPLGTTPVSVSQLCKFRGRITSGQRVLNLTELDGSPFMAFAAPAPAGFPDLGSCDWHIEMSKIPNSSTQNNPIVVNSVKPNSQQFVPHLSSITLDDNVSSGGDYIGTIQWTSPPSDSGGANTNFWKIPDYGSSLAEASQLAPAVYPPGFNEVIVYFMASIPGPNQSGSPNLVPCLLPQEYITHFISEQAPIQGEAALLHYVDPDTNRNLGEFKLYPGGYLTCVPNSSSTGPQQLPLDGVFVFASWVSRFYQLKPVGTAGPARGRLGVRR

SEQ ID NO: 5 - protein, 543 aa, Norovirus norwalk virus
  source 1..543: note = strain Hu/GI.5/Siklos-HUN5407/2013/HUN
MMMASKDAPSSADGANGAGQLVPEVNNAEPLPLDPVAGASTALATAGQVNMIDPWIFNNFVQAPQGEFTISPNNTPGDILFDLQLGPHLNPFLAHLSQMYNGWVGNMRVRVILAGNAFTAGKVIICCVPPGFQSRTLSIAQATLFPHIIADVRTLEPIEIPLEDVRNTLYHTNDNQPTMRLLCMLYTPLRTGGGSGGTDAFVVAGRVLTCPSSDFNFLFLVPPTVEQKTRPFSVPNIPLQLLSNSRVPNLIQSMVLSPDQAQNVQFQNGRCTTDGQLLGTTPVSVSQILKFRGKVSAGSKVINLTELDGSPFLAFEAPAPTGFPDLGTSDWHVEMSLNSNSQSSGNPILLRDIHPNSSEFVPHLGSVCVTAAIEVAGDYTGTIQWTSQPSNVTPVPDVNFWTIPHYGSNLAEASQLAPVVYPPGFGEAIVYFMSPIPGPNTAHKPNLVPCLLPQEFVTHFVSEQAPSMGEAALVHYVDPDTNRNLGEFKLYPEGFITCVPNGTGPQQLPLNGVFVFASWVSRFYQLKPVGTASSARGRLGVRR

SEQ ID NO: 6 - protein, 540 aa, Norovirus norwalk virus
  source 1..540: note = strain Hu/GI.6/TCH-099/USA/2003
MMMASKDAPTSPDGASGAGQLVPEANTAEQISMDPVAGASTAVATAGQVNMIDPWIFNNFVQAPQGEFTISPNNTPGDILFDLQLGPHLNPFLAHLSQMYNGWVGNMRVRILLAGNAFTAGKIIICCVPPGFDARILTIAQATLFPHLIADVRTLEPVELPLEDVRNVLYHNSSQPQPTMRLVAMLYTPLRTGGGSGGTDAFVVAGRVLTCPAPDFSFLFLVPPSVEQKTRVFSVPNIPLKDLSNSRVPVPVQGMFMSPDVNQSVQFQNGRCQIDGQLQGTTPVSLSQLCKIRGKTSSNARVLNLSEVDGTPFIPLESPAPVGFPDLGGCDWHVNFTFQAQDQDPSQSVTFATNDASFVPYLGSISPHNGGDFHAGDIIGSLGWISAPSGNSQLNVWAIPKYGSSLPDVTHLAPAVFPPGFGEVILYFYSTFPGSGQPSQLQVPCLLPQEFITHFCNEQAPIAGEAALLHYVDPDTGRNLGEFKLYPDGFMTCVPNSVSSGPQTLPINGVFVFVSWVSRFYQLKPVGTASAARRLGLRRI

SEQ ID NO: 7 - protein, 538 aa, Norovirus norwalk virus
  source 1..538: note = strain Hu/GI.7/Providence191/2010/USA
MMMASKDAPSNMDGTSGAGQLVPEVNTAEPLPLEPVVGAATAAATAGQVNLIDPWIMNNFVQAPEGEFTISPNNTPGDILFDLRLGPHLNPFLLHLSQMYNGWVGNMRIRVMLAGNAFSAGKIIICCVPPGFESQNISIGQATMFPHVIADVRVLEPIEIPLDDVRNVLYHTNENRPTMRLLCMLYTPLRAGGASSGTDPFVIAGRVLTCPAPDFNFLFLVPPSVEQKTRQLTIPNIPLNNLANSRVPAMINKMAVSADQNQVVQFQNGRCTLEGQLLGTTPVSANQVARIRGKVFSTNSGTGLNLTETDGTPYHAFESPAPLGFPDIGDCDWHVYAFKVNQNTGDPMYRLDITQGNSFAPHLGSIEFTSDQHPSGDQLGTLTWISPLNNASRVDPWKIPSYGSTLTESTNLAPPIFPPGFGEAIVYFMSDFPIVSGNTTQIPCTLPQEFISSFVEQQAPIRGEAALLHYVDPDTHRNLGEFKLYPDGFITCVPNTGGGPQNLPSNGVFIFSSWVSRYYQLKPVGTAGPARRLGVRRI

SEQ ID NO: 8 - protein, 535 aa, Norovirus norwalk virus
  source 1..535: note = strain Hu/GII.1/Ascension208/2010/USA
MKMASNDAAPSNDGAAGLVPEVNNETMALEPVAGASIAAPLTGQNNVIDPWIRMNFVQAPNGEFTVSPRNSPGEVLLNLELGPELNPFLAHLSRMYNGYAGGVEVQVLLAGNAFTAGKLVFAAIPPHFPLENLSPGQITMFPHVIIDVRTLEPVLLPLPDVRNNFFHYNQQPEPRMRLVAMLYTPLRSNGSGDDVFTVSCRVLTRPSPDFDFNYLVPPTVESKTKPFTLPILTIGELSNSRFPVPIDELYTSPNEGVVVQPQNGRSTLDGELLGTTQLVPSNICALRGRINAQVPDDHHQWNLQVTNANGTSFDPTEDVPAPLGTPDFLANIYGVTSQRNPDNTCRAHDGVLATWSPKFTPKLGSVVLGTWEESDLDLNQPTRFTPVGLYDTGHFDQWVLPNYSGRLTLNMNLAPSVAPLFPGEQILFFRSHIPLKGGTSNGAIDCLLPQEWIQHFYQESAPSPTDVALIRYTNPDTGRVLFEAKLHRQGFITVANSGSRPIVVPPNGYFRFDSWVNQFYSLAPMGTGNGRRRVQ

SEQ ID NO: 9 - protein, 542 aa, Norovirus norwalk virus
  source 1..542: note = strain Hu/GII.2/OH10026/2010/JP
MKMASNDAAPSTDGAAGLVPESNNEVMALEPVAGAALAAPVTGQTNIIDPWIRANFVQAPNGEFTVSPRNSPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEVISVQCQNGRCTLDGELQGTTQLQVSGICAFKGEVTAHLHDNDHLYNVTITNLNGSPFDPSEDIPAPLGVPDFQGRVFGVISQRDKHNTPGHNEPANRAHDAVVPTYTAQYTPKLGQIQIGTWQTDDLTVNQPVNFTPVGLSDTDHFNQWVVPRYAGALNLNTNLAPSVAPVFPGERLLFFRSYIPLKGGYGTPAIDCLLPQEWVQHFYQEAAPSMSEVALVRYINPDTGRALFEAKLHRAGFMTVSSNTSAPVVVPANGYFRFDSWVNQFYSLAPMGTGNGRRRIQ

SEQ ID NO: 10 - protein, 549 aa, Norovirus norwalk virus
  source 1..549: note = strain Hu/GII.3/NIHIC8.1/2011/USA
MKMASNDAAPSNDGAAGLVPEINSEAMALEPVAGAAIAAPLTGQQNIIDPWIMNNFVQAPGGEFTISPRNGPGEVLLNLELGPEINPYLAHLARMYNGYAGGFEVQVVLAGNAFTAGKIIFAAIPPNFPIDNLSAAQVTMCPHVIVDVRQLEPVNLPMPDVRNSFFHYNQGTDSRLRLIAMLYTPLRANNSGDDVFTVSCRVLTRPSPDFSFNFLVPPTVESKTKPFTLPILTISEMSNSRFPVPIDSLHTSPTDNIVVQCQNGRVTLDGELMGTTQLLPSQICAFRGVLTQRTSRASDQADTAVTPRAFDHRWHIQLDNLNGTHYDPSEDIPGPLGIPDFRGRVYGVASQRNSDGSTRAHEAKIDTTSSRFTPKLGSLEISTESDDFDENKPTRFTPVGIGVDNEREFQQWSLPDYSGQFTHNMHLAPAVAPNFPGEQLLFFRSHLPSSGGWVDGTLDCLLPQEWIQHFYQESAPSQTQVALVRYVNPDTGRVLFEAKLHKSGFMTIARSGDSPITVPPNGYFRFESWVNPFYTLAPMGTGNGRRRIQ

SEQ ID NO: 11 - protein, 540 aa, synthetic construct
  REGION 1..540: note = GII.4/Consensus VP1 sequence
MKMASSDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTQEYTMNLASQNWNNYDPTEEIPAPLGTPDFVGKIQGVLTQTTRGDGSTRGHKATVSTGSVHFTPKLGSVQFSTDTSNDFETGQNTKFTPVGVVQDGSTTHQNEPQQWVLPDYSGRDSHNVHLAPAVAPTFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPMGNGTGRRRAL

SEQ ID NO: 12 - protein, 540 aa, Norovirus norwalk virus
  source 1..540: note = strain Hu/GII.4/Sydney/NSW0514/2012/AU
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 13 - protein, 540 aa, Norovirus norwalk virus
  source 1..540: note = strain Hu/GII.4/Yerseke38/2006/NL
MKMASSDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFSVPILTVEEMTNSRFPIPLEKLFTGPSSAFVVQPQNGRCTTDGVLLGTTQLSPVNICTFRGDVTHIAGTQEYTMNLASQNWNNYDPTEEIPAPLGTPDFVGKIQGVLTQTTRRDGSTRGHKATVSTGSVHFTPKLGRIQFSTDTSNDFETGQNTRFTPVGVVQDGSTTHQNEPQQWVLPDYSGRDSHNVHLAPAVAPSFPGEQLLFFRSTMPGCSGYPNMNLDCLLPQEWVQHFYQEAAPAQSDVALLRFVNPDTGRVLFECKLHKSGYVTVAHTGQHDLVIPPNGYFRFDSWVNQFYTLAPMGNGTGRRRAL

SEQ ID NO: 14 - protein, 540 aa, Norovirus norwalk virus
  source 1..540: note = strain Hu/GII.4/New Orleans1805/2009/USA
MKMASSDANPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHIPGSRNYTMNLASQNWNSYDPTEEIPAPLGTPDFVGKIQGVLTQTTRTNGSTRGHKATVYTGSADFSPKLGRVQFATDTDNDFETNQNTKFTPVGVIQDGSTTPRNEPQQWVLPSYSGRNIHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAL

SEQ ID NO: 15 - protein, 540 aa, Norovirus norwalk virus
  source 1..540: note = strain Hu/GII.4/DenHaag89/2006/NL
MKMASNDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGEDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGSRNYTMNLASLNWNNYDPTEEIPAPLGTPDFVGKIQGVLTQTTKGDGSTRGHKATVYTGSAPFTPKLGSVQFSTDTENDFETHQNTKFTPVGVIQDGSTTHRNEPQQWVLPSYSGRNVHNVHLAPAVAPTFPGEQLLFFRSTMPGCSGYPNMDLDCLLPQEWVQHFYQEAAPAQSDVALLRFVNPDTGRVLFECKLHKSGYVTVAHTGQHDLVIPPNGYFRFDSWVNQFYTLAPMGNGTGRRRAL

SEQ ID NO: 16 - protein, 540 aa, Norovirus norwalk virus
  source 1..540: note = strain Hu/GII.4/Houston/TCH186/2002/US
MKMASSDASPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLRANNAGDDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTHDYTMNLASQNWNNYDPTEEIPAPLGTPDFVGKIQGVLTQTTRGDGSTRGHKATVSTGSVHFTPKLGSVQFTTDTNNDLETGQNTKFTPVGVVQDGNSAHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYVTVAHTGPHDLVIPPNGYFRFDSWVNQFYTLAPMGNGAGRRRAL

SEQ ID NO: 17 - protein, 547 aa, Norovirus norwalk virus
  source 1..547: note = strain Hu/GII.6/Ehime120246/2012/JP
MKMASNDAAPSNDGAANLVPEANNEVMALEPVVGASIAAPVVGQQNIIDPWIRENFVQAPQGEFTVSPRNSPGEMLLNLELGPELNPYLSHLSRMYNGYAGGMQVQVVLAGNAFTAGKIIFAAVPPHFPVENISAAQITMCPHVIVDVRQLEPVLLPLPDIRNRFFHYNQENTPRMRLVAMLYTPLRANSGEDVFTVSCRVLTRPAPDFEFTFLVPPTVESKTKPFTLPILTLGELSNSRFPAPIDMLYTDPNEGIVVQPQNGRCTLDGTLQGTTQLVPTQICAFRGTLIGQTSRSSDSTDSAPRRRDHPLHVQLKNLDGTQYDPTDEVPAVLGAIDFKGTVLGVASQRDVSGQQVGATRAHEVHINTTDPRYTPKLGSILMHSESDDFVTGQPVRFTPIGMGDNDWHQWELPDYSGHLTLNMNLAPAVAPAFPGERILFFRSIVPSAGGYGSGQIDCLIPQEWVQHFYQEAAPSQSAVALIRYVNPDTGRNIFEAKLHREGFITVANSGNNPIVVPPNGYFRFEAWVNQFYTLTPMGTGQGRRRNQ

SEQ ID NO: 18 - protein, 540 aa, Norovirus norwalk virus
  source 1..540: note = strain Hu/GII.7/NSW088L/2007/AUS
MKMASNDAAPSNDGAAGLVPEINNEVMPLEPVAGASLATPVVGQQNIIDPWIRNNFVQAPAGEFTVSPRNSPGEILLDLELGPELNPYLAHLARMYNGHAGGMEVQIVLAGNAFTAGKIIFAAIPPGFPYENLSPSQITMCPHVIIDVRQLEPVLLPMPDIRNNFFHYNQGNDPKLRLIAMLYTPLRANNSGDDVFTVSWRGLTKTSPDFEFTFLVPPTVESKTKQFTLPILKISEMTNSRFPVPVEMMYTARNENQVVQPQNGRVTLDGELLGTTPLLAVNICKFKGEVIAKNGDVRSYRMDMEITNTDGTPIDPTEDTPGPIGSPDFQGILFGVASQRNKNEQNPATRAHEANINTGGDQYAPKLAQVKFFSESQDFEVHQPTVFTPVGVAGDTSHPFRQWVLPRYGGHLTNNTHLAPAVAPLFPGEQILFFRSQIPSSGGHELGYMDCLVPQEWVQHFYQEAATAQSEVALIRFINPDTGRVLFEAKLHKQGFITVAHTGDNPIVMPPNGYFRFEAWVNQFYSLAPVGTGNGRRRIQ

SEQ ID NO: 19 - protein, 537 aa, Norovirus norwalk virus
  source 1..537: note = strain Hu/GII.12 strain E5152
MKMASNDAAPSNDGAAGLVPEVNNETMALEPVAGASIAAPLTGQNNVIDPWIRLNFVQAPNGEFTVSPRNSPGEVLLNLELGPELNPYLAHLSRMYNGYAGGVEVQVLLAGNAFTAGKLVFAAVPPHFPLENISPGQITMFPHVIIDVRTLEPVLLPLPDVRNNFFHYNQQNEPRMRLVAMLYTPLRSNGSGDDVFTVSCRVLTRPSPDFDFNYLVPPTVESKTKPFTLPILTIGELTNSRFPVPIDELYTSPNESLVVQPQNGRCALDGELQGTTQLLPTAICSFRGRINQKVSGDNHVWNMQVTNINGTPFDPTEDVPAPLGTPDFSGKLFGVLSQRDHDNACRSHDAVIATDSAKFTPKLGAIQIGTWEEDDVHINQPTKFTPVGLFEREGFNQWTLPNYSGALTLNMGLAPPVAPTFPGEQILFFRSHIPLKGGVADPVIDCLLPQEWIQHLYQESAPSQTDVALIRFTNPDTGRVLFEAKLHRSGYITVANTGSRPIVVPANGYFRFDSWVNQFYSLAPMGTGNGRRRVQKG

SEQ ID NO: 20 - protein, 540 aa, Norovirus norwalk virus
  source 1..540: note = strain Hu/GII.17/C142/GF/1978
MKMASNDAAPSNDGATGLVPEINHETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYNNQPANRMRLVAMLYTPLRSNGSGDDVFTVSCRVLTRPTPDFEFTYLVPPSVESKTKPFSLPILTISELTNSRFPAPIDSLYTAQNNNLNVQCQNGRCTLDGELQGTTQLLPSGICAFRGKLTADVHQSHDDRWHMQLTNLNGTPFDPTEDVPAPLGTPDFTGLLFGVASQRNVVSNPNTTRAHEAVISTTSSQFVPKLGSINFGSTSDDFQLQQPTKFTPVGIKVESGHDFDQWALPRYSGHLTLNMNLAPPVAPNFPGEQLLFFRSNVPCAGGVSDGVIDCLLPQEWIQHFYQESAPSQSDVALIRYVNPDTGRTLFEAKLHRTGYITVAHSGDYPLVVPSNGYFRFDSWVNQFYSLAPMGTGNGRRRVQ

SEQ ID NO: 21 - protein, 538 aa, Norovirus norwalk virus
  source 1..538: note = strain Hu/GII.17/JP/2014/Nagano7-1
MKMASNDAAPSNDGAAGLVPEGNNETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYNNQPNSRMRLVAMLYTPLRSNGSGDDVFTVSCRVLTRPTPDFEFTYLVPPSVESKTKPFSLPILTLSELTNSRFPVPIDSLFTAQNNVLQVQCQNGRCTLDGELQGTTQLLPSGICAFRGRVTAETDHRDKWHMQLQNLNGTTYDPTDDVPAPLGTPDFKGVVFGVASQRNVGNDAPGSTRAHEAVISTYSPQFVPKLGSVNFRSNDNDFQLQPTKFTPVGINDDGDHPFRQWELPDYSGLLTLNMNLAPPVAPNFPGEQLLFFRSFVPCSGGYNQGIVDCLIPQEWIQHFYQESAPSQSDVALIRYVNPDTGRTLFEAKLHRSGYITVAHSGDYPLVVPANGYFRFDSWVNQFYSLAPMGTGNGRRRAQ

SEQ ID NO: 22 - protein, 540 aa, Norovirus norwalk virus
  source 1..540: note = strain Hu/GII.17/HKG/2015/CUHK-NS-513
MKMASNDAAPSNDGAAGLVPEGNNETLPLEPVAGAAIAAPVTGQNNIIDPWIRTNFVQAPNGEFTVSPRNSPGEILLNLELGPDLNPYLAHLSRMYNGYAGGVEVQVLLAGNAFTAGKILFAAVPPNFPVEFLSPAQITMLPHLIVDVRTLEPIMIPLPDVRNTFFHYSNQPNSRMRLVAMLYTPLRSNGSGDDVFTVSCRVLTRPTPDFEFTYLVPPSVESKTKPFSLPILTLSELTNSRFPVPIDSLFTAQNNVLQVQCQNGRCTLDGELQGTTQLLPSGICAFRGRVTAQINQRDRWHMQLQNLNGTTYDPTDDVPAPLGTPDFKGVVFGMVSQRNVGNDAPGSTRAQQAWVSTYSPQFVPKLGSVNLRISDNDDFQFQPTKFTPVGVNDDDDGHPFRQWELPNYSGELTLNMNLAPPVAPNFPGEQLLFFRSFVPCSGGYNQGIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 23 - DNA, 372 bp, synthetic construct
  misc_feature 1..372: note = DNA sequence of variable heavy chain of mAb 1227
  source 1..372: mol_type = other DNA
caagtacaattggtgcaaagtggaggtggaatggtacaacccggtggttcactgagcctctcttgcgccgcatctggatttacgctctccaactacgccatgacatgggtacgccaggcgccggggaaaggtctcgaatgggtctcctcaataggtggctccggtggaactacctactatgccgatagcgttaagggacgcttcactatcagccgagattcctctatgaacaccctgtatttgcaaatgtcaaacctccgagcgggcgacaccgcagtttactattgtgctaaggataaaacgcgcactctccgccttggctatagtggtatggacgtgtggggacagggaactacagttacagttagttca

SEQ ID NO: 24 - DNA, 319 bp, synthetic construct
  misc_feature 1..319: note = DNA sequence of variable light chain of mAb 1227
  source 1..319: mol_type = other DNA
gatatacgcctgacgcaaagcccgtcatccctttccgcttcagtgggcgatagagtaacgataacctgtagggcaagccaatccataagctcttaccttaactggtatcagcagaaaccgggcaaggctcccgacctccttatatacggggcatcctcacttcagtccggcgtcccttcaagatttagtgggtcagggtccggcacggactttacattgacgatatcctcattgcagcctgaggatttcggcaactactattgtcagcagtcattctcaacgccgagaacatttggacaaggtacgaaagttgagctga

SEQ ID NO: 25 - DNA, 378 bp, synthetic construct
  misc_feature 1..378: note = DNA sequence of variable heavy chain of mAb 1431
  source 1..378: mol_type = other DNA
caagtgcaactggtccagtctggtgggggccttgtccagccaggtgggtcacttagactttcctgcgctgccagcggttttgcattcagtaatcacggtatgcactgggtacgccaagcgcccggcaaaggcctggagtggctctcctatatatctggttcaacaggcgcgattcattacgctaactcagtaaaagggaggtttacaatctcaagagacaacgcaaggaattccctggatttgcagatgaatagcctgggggacgaggacaccgcagtttactactgtgcccgcgacggaccgcgccccgacggtacgggctacgccggcccttctaacgactattggggtcagggtactctggtctccgtgagtagc

SEQ ID NO: 26 - DNA, 321 bp, synthetic construct
  misc_feature 1..321: note = DNA sequence of variable light chain of mAb 1431
  source 1..321: mol_type = other DNA
gaaatcgtcctgacccaatcaccagcatccctgtctcttagtccaggcgaaagagcgaccctgtcttgtaaggcttctaggagtatttccatttacctcgcgtggtatcaacagaagccgggccaagctccgaggcttttgatttatgatgcctcctaccgcgcaatcggaataccggcaaggtttagtggaagtggttccggtactgatttcactttgaccatatctaaccttgagcctgaagattttgcggtgtactactgtcagcatcggagttcctggccagctttgaccttcggaggggggacgaaggtcgaaatt

SEQ ID NO: 27 - protein, 124 aa, synthetic construct
  REGION 1..124: note = Amino acid sequence of variable heavy chain of mAb 1227
QVQLVQSGGGMVQPGGSLSLSCAASGFTLSNYAMTWVRQAPGKGLEWVSSIGGSGGTTYYADSVKGRFTISRDSSMNTLYLQMSNLRAGDTAVYYCAKDKTRTLRLGYSGMDVWGQGTTVTVSS

SEQ ID NO: 28 - protein, 106 aa, synthetic construct
  REGION 1..106: note = Amino acid sequence of variable light chain of mAb 1227
DIRLTQSPSSLSASVGDRVTITCRASQSISSYLNWYQQKPGKAPDLLIYGASSLQSGVPSRFSGSGSGTDFTLTISSLQPEDFGNYYCQQSFSTPRTFGQGTKVEL

SEQ ID NO: 29 - protein, 126 aa, synthetic construct
  REGION 1..126: note = Amino acid sequence of variable heavy chain of mAb 1431
QVQLVQSGGGLVQPGGSLRLSCAASGFAFSNHGMHWVRQAPGKGLEWLSYISGSTGAIHYANSVKGRFTISRDNARNSLDLQMNSLGDEDTAVYYCARDGPRPDGTGYAGPSNDYWGQGTLVSVSS

SEQ ID NO: 30 - protein, 107 aa, synthetic construct
  REGION 1..107: note = Amino acid sequence of variable light chain of mAb 1431
EIVLTQSPASLSLSPGERATLSCKASRSISIYLAWYQQKPGQAPRLLIYDASYRAIGIPARFSGSGSGTDFTLTISNLEPEDFAVYYCQHRSSWPALTFGGGTKVEI